(19) United States
(12) Patent Application Publication
(10) Pub. No.: US 2003/0082194 A1
(43) Pub. Date: May 1, 2003

(54) Compositions and methods for diagnosis and therapy of malignant mesothelioma

(76) Inventors: Alexander Gaiger, Seattle, WA (US); Martin A. Cheever, Mercer Island, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/791,477

(22) Filed: Feb. 22, 2001

Related U.S. Application Data
(60) Provisional application No. 60/184,070, filed on Feb. 22, 2000.

(51) Int. Cl. A61K 39/00; A61K 39/38; A61K 48/00

(52) U.S. Cl. 424/184.1; 514/44

(57) ABSTRACT

Disclosed are compositions and methods for the diagnosis and therapy of Wilms' tumor antigen-associated cancers, and in particular, malignant pleural mesothelioma. In particular embodiments, the invention provides new, effective methods, compositions and kits for eliciting immune and T cell response to Wilms' tumor antigen polypeptide-derived antigenic fragments, and methods for the use of such compositions for diagnosis, detection, treatment, monitoring, and/or prevention of human malignant pleural mesothelioma

### 1. BACKGROUND OF THE INVENTION

The present application claims priority to United States Provisional Patent Application Ser. No. 60/184,070, filed Feb. 22, 2000; the entire specification, claims and figures of which are incorporated herein by reference without disclaimer. Portions of this research were conducted in part through funding from the United States Department of Health and Human Services under grant number SBIR R43 CA81752.

### 1.1 FIELD OF THE INVENTION

The present invention relates generally to the fields of cancer diagnosis and therapy. More particularly, it concerns the surprising discovery of compositions and methods for the detection and immunotherapy of mesotheliomas, and particularly, malignant pleural mesothelioma. The invention provides new, effective methods, compositions and kits for eliciting immune and T cell response to Wilms' tumor antigen polypeptide-derived antigenic fragments, and methods for the use of such compositions for diagnosis, detection, treatment, monitoring, and/or prevention of human malignant pleural mesothelioma.

### 1.2 DESCRIPTION OF RELATED ART

1.2.1 Wilms' Tumorantigen

The Wilms' tumor gene encodes a nuclear-expressed polypeptide designated WT1, which is possesses the structural features of a DNA binding transcription factor. WT1 has alternatively spliced variants, including a 429-amino acid polypeptide comprising four contiguous zinc finger domains at its carboxy terminus, and a glutamine/proline-rich region at its amino terminus, that mediates transcriptional suppression or activation in transient transfection assays.

A variety of diagnostic reagents for the detection of WT1 peptides exist, including rabbit polyclonal sera that specifically recognize large internal amino acid fragments of the wild type WT1 polypeptide. Commercially available WT1 polyclonal antibodies exist, but they have particular disadvantages including cross-reactivity with closely related proteins, and inconsistent results in antigen specificity and binding affinity studies, because of their nature as polyclonal sera. Such sera are therefore not particularly desirable for diagnostic uses, and are not useful for developing therapeutic reagents for in vivo inhibition of WT1 polypeptide.

Commercially-available mouse monoclonal antibody have also been reported, however most are unsuitable for most therapeutic and diagnostic applications because they either (a) recognize only particular unique splice variant sequences (which are expressed in only a subpopulation of the alternatively-spliced WT1 mRNA); or (b) broadly cross-react with homologous, but functionally unrelated peptides, polypeptides, or proteins.

1.2.2 Detection of WT1 Polypeptides in Malignant Mesothelioma

Malignant pleural mesothelioma is an increasingly common cancer, caused primarily by exposure to asbestos. The millions of workers who were exposed to asbestos dust prior to the implementation of asbestos regulation and improved control measures are at risk for the disease. In addition, workers continue to be exposed to significant amounts of asbestos, when asbestos materials are disturbed during renovation, repair or demolition. Asbestos-containing materials continue to be found in industrial, commercial and residential settings throughout the U.S., resulting in a sizeable population that remains at risk for malignant mesothelioma.

The prognosis for malignant mesothelioma is influenced by the stage of the disease. Surgery, as well as adjuvant immunological treatments (e.g., interferon or interleukin) can be effective treatment, but only in the rare event of an early stage diagnosis.

### 1.3 DEFICIENCIES IN THE PRIOR ART

A major obstacle to contemporary cancer treatment is the problem of selectivity; that is, the ability to inhibit the multiplication of cancerous cells, while leaving unaffected the function of normal cells. Unfortunately, most mesothelioma patients are diagnosed only in advanced stages, where neither radiation, nor chemotherapy, nor multimodality treatments can significantly alter the poor prognosis. Moreover, the absence of a standard effective therapy for these patients makes long-term survival unlikely (Von Bultzingslowen, 1999; Gennaro et al., 2000).

The poor survival rate for patients afflicted with malignant mesothelioma, however, could be greatly improved by diagnostic methods that provide more accurate and earlier detection, as well as improved therapies that selectively inhibit the hyperproliferating meothelioma cells. The need also exists for effective treatment regimens for mesotheliomas, and in particular, human malignant pleural mesothelioma, that circumvent the toxic side effects of existing therapies and provide more specific gene expression of the therapeutic constructs directly in the cancerous cells. Development of suitable treatment regimens for human malignant pleural mesothelioma would represent a significant advance for those of skill in the oncologic arts, and would facilitate improved diagnostic and therapeutic modalities for this aggressive cancer.

### 2. SUMMARY OF THE INVENTION

The present invention addresses the foregoing long-felt need and other deficiencies in the art by identifying new and effective strategies for the diagnosis, detection, prophylaxis, therapy, and immunomodulation of WT1-associated cancers, and in particular, malignant pleural mesothelioma. The present invention is based, in part, upon the surprising and unexpected discovery that immune and T cell responses to particular antigenic peptide fragments of the Wilms' tumor (WT) gene product (e.g., WT1) can provide particularly advantageous compositions and methods for the diagnosis, prophylaxis and/or therapy for an animal having, suspected of having, or at risk for developing one or more malignant diseases characterized by increased WT1 gene expression, and in particular, malignant pleural mesothelioma in a human. The WT1 gene was originally identified and isolated on the basis of a cytogenetic deletion at chromosome 11p13 in patients with Wilms' tumor (U.S. Pat. No. 5,350,840). The gene consists of 10 exons and encodes a zinc finger transcription factor, and sequences of mouse and human WT1 polypeptides are provided in FIG. 1 (SEQ ID NO:319 and SEQ ID NO:320, respectively).

In a first embodiment, the invention provides a method of generating an immune or a T cell response in an animal, and in particular in a mammal such as a human. The method concerns in a general sense the administration of at least a first composition to the animal that comprises at least a first isolated peptide of from 9 to about 60 amino acids in length, or at least a first nucleic acid segment that encodes such a peptide, wherein the peptide comprises a first contiguous amino acid sequence according to any one of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326, and more particularly, a contiguous amino acid sequence according to any one of SEQ ID NO:28 through SEQ ID NO:318, with peptides comprising one or more of the primary amino acid sequences disclosed in SEQ ID NO:2, SEQ ID NO:34, SEQ ID NO:35, SEQ ID NO:49, SEQ ID NO:88, SEQ ID NO:144, SEQ ID NO:147, SEQ ID NO:185, SEQ ID NO:198, SEQ ID NO:199, SEQ ID NO:255, SEQ ID NO:282, SEQ ID NO:283, and SEQ ID NO:293 being particularly preferred.

The invention encompasses peptides that may be of any intermediate length in the preferred ranges, such as for example, those peptides of about 55, about 50, about 45, about 40, about 35, about 30, about 25, about 20, or even about 15 amino acids or so in length, as well as those peptides having intermediate lengths including all integers within these ranges (e.g., the peptides may be about 54, about 53, about 52, about 51, about 49, about 48, about 47, about 46, about 44, about 43, about 42, about 41, about 39, about 38, about 27, or even about 36 or so amino acids in length, etc.). In particular embodiments, when smaller peptides are preferred, the length of the peptide may be 9, or about 10, or about 11, or about 12, or about 13, or about 14 or even about 15 or so amino acids in length, so long as the peptide comprises at least a first contiguous amino acid sequence according to any one of SEQ ID NO:1, SEQ ID NO:2, SEQ ID NO:3, SEQ ID NO:4, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, as well as any one of SEQ ID NO:28 to SEQ ID NO:311, SEQ ID NO:313, SEQ ID NO:314, SEQ ID NO:316 to SEQ ID NO:318, SEQ ID NO:321, SEQ ID NO:322, SEQ ID NO:323, SEQ ID NO:324, SEQ ID NO:325, and SEQ ID NO:326. Likewise, when slightly longer peptides are preferred, the length of the peptide may be about 16, or about 17, or about 18, or about 19, or about 20, or about 21, or about 22, or about 23, or about 24, or even about 25 or so amino acids in length, so long as the peptide comprises at least a first contiguous amino acid sequence according to any one of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318 and SEQ ID NO:321 to SEQ ID NO:326. When intermediate-length antigenic peptides or antigen binding fragments are desired, the peptides may be on the order of about 26, or about 27, or about 28, or about 29, or about 30, or about 31, or about 32, or about 33, or about 34, or even about 35 or so amino acids in length, so long as they each comprise at least a first contiguous amino acid sequence according to any one of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326.

These peptides comprise at least a first contiguous amino acid sequence according to any one of SEQ ID NO:1 to SEQ ID NO:4, SEQ ID NO:13 to SEQ ID NO:20, SEQ ID NO:28 to SEQ ID NO:318, and SEQ ID NO:321 to SEQ ID NO:326, but may also, optionally comprise at least a second, at least a third, or even at least a fourth or greater contiguous amino acid sequence according to any one of SEQ ID NO: 1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326. A single peptide may contain only one of the contiguous amino acid sequences disclosed herein, or alternatively, a single peptide may comprise a plurality of contiguous amino acid sequences according to any one of SEQ ID NO: 1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326. In fact, the peptide may comprise a plurality of the same contiguous amino acid sequences, or they may comprise one or more different contiguous amino acid sequences disclosed in SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326. For example, a single peptide of from 9 to about 50 amino acids in length could comprise a single epitopic peptide disclosed herein, or could comprise 2, 3, 4, or even 5 distinct epitopic sequences as disclosed in any of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326. Alternatively, a single peptide of from 9 to about 50 amino acids in length could comprise 2, 3, 4, or even 5 identical epitopic sequences as disclosed in any one of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326.

In one exemplary embodiment, the peptide composition comprises at least a first isolated peptide of from 9 to about 40 amino acids in length, or at least a first nucleic acid segment that encodes such a peptide; wherein the peptide comprises at least a first contiguous amino acid sequence selected from the group consisting of SEQ ID NO:34, SEQ ID NO:35, SEQ ID NO:49, SEQ ID NO:88, SEQ ID NO:144, SEQ ID NO:147, SEQ ID NO:185, SEQ ID NO:198, SEQ ID NO:199, and SEQ ID NO:282.

Preferred peptides of the present invention likewise encompass those from 10 to about 60 amino acids in length, those from 15 to about 60 amino acids in length, those from 12 to about 60 amino acids in length, those from 13 to about 60 amino acids in length, as well as those from 14 to about 60 amino acids in length, and those from 15 to about 60 amino acids in length. Likewise, preferred peptides of the present invention encompass those from 16 to about 60 amino acids in length, and any and all lengths, and sub-ranges of lengths within the overall preferred range of peptides of from 9 to about 60 amino acids or so in length. In similar fashion, the invention also encompasses those peptides having a length of from 10 or 11 to about 55 or 60 amino acids in length, and those having a length of from 12 or 13 to about 45 or 50 amino acids in length, as well as those peptides having a length of from 14 or 15 to about 35 or 40 amino acids in length, those peptides having a length of from 16 or 17 to about 25 or 30 amino acids in length, and those peptides having a length of from 18 or 19 to about 20 or so amino acids in length, and so on, to include all sub-ranges within the overall range of from 9 to about 60 amino acids in length.

Throughout this disclosure, a phrase such as “a sequence as disclosed in SEQ ID NO:1 to SEQ ID NO:4” is intended to encompass any and all contiguous amino acid sequences disclosed by any of these sequence identifiers, and particularly, the peptide sequences disclosed in Table 2 through Table 49 of the present specification. That is to say, “a sequence as disclosed in any of SEQ ID NO:1 through SEQ ID NO:4” means a sequence that is disclosed in SEQ ID NO:1, SEQ ID NO:2, SEQ ID NO:3, or SEQ ID NO:4. Likewise, “SEQ ID NOs:25 to 37” means any and all such sequences as disclosed in SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, and SEQ ID NO:37, and so forth. In fact, the invention encompasses peptides and polynucleotides encoding them that comprise at least a first contiguous amino acid sequence as disclosed in any one of the sequences identified as SEQ ID NO: 1 to SEQ ID NO:4, SEQ IDNO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326.

The invention also encompasses polynucleotides that comprise at least a first sequence region that encodes one or more of the peptides or peptide variants as disclosed herein. Such polynucleotides may comprise a sequence region of 27 to about 5000 nucleotides in length, or a sequence region of 27 to about 2000 nucleotides in length, or a sequence region of 27 to about 1000 nucleotides in length, or a sequence region of 27 to about 900, or about 800, or about 700, or about 600, or about 500, or about 400, or about 300, or about 200, or even about 100 or so nucleotides in length.

As in the case of the peptides, the length of the sequence region that encodes the peptide may be of any intermediate length in these ranges, such as those polynucleotides that comprise at least a first sequence region of from about 30 to about 750 nucleotides in length, those that comprise at least a first sequence region of from about 35 to about 650 nucleotides in length, and those that comprise at least a first sequence region of from about 40 to about 550, about 450, about 350, about 250, about 150, or even about 50 or so nucleotides in length. Such sequence regions may be on the order of about 27, or about 28, or about 29, or about 30, or about 31, or about 32, or about 33, or about 34, or even about 35 or so nucleotides in length, so long as the sequence region encodes at least a first peptide that comprises at least a first contiguous amino acid sequence according to any one of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326. When intermediate-length antigenic peptides or antigen binding fragments are desired, the nucleic acids that encode them may be on the order of about 40, about 45, or about 50, or about 55, or about 60, or about 65, or about 70, or about 75, or about 80, or even about 85 or 90 or so nucleotides in length, so long as they each encode at least a first peptide that comprises at least a first contiguous amino acid sequence according to any one of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326. When polynucleotides are contemplated that comprise sequence regions encoding larger antigenic peptides or antigen-binding fragments, the nucleic acid sequence region encoding them will necessarily be longer in length. For example, a nucleic acid sequence region encoding a peptide or antigen binding fragment on the order of about 40 to 50 amino acids in length, will necessarily be at least from about 120 to about 150 or so nucleotides in length, given the fact that a triplet codon is required to encode a single amino acid.

Likewise, the polynucleotides comprising such sequence regions can be substantially larger than the coding region itself, particularly when the sequence region is operably linked to one or more promoters, or to one or more sequence regions that encode one or more signal sequences, and/or peptide fusion products. In those embodiments, the polynucleotide may be on the order of about 500, about 600, about 700, about 800, about 900, about 1000, about 1100, about 1200, about 1300, about 1400, or even about 1500, 1600, 1700, 1800, 1900, or even 2000 or so nucleotides in length, even up to and including those sequences that are on the order of about 10,000 or so nucleotides in length. Such polynucleotides are particularly useful in the preparation of expression vectors, delivery vehicles, viral vectors, and transformed host cells that express the particular encoded peptide(s) and/or antigen-binding fragment(s) encoded by the sequence region comprised within the polynucleotide and/or genetic construct or expression element.

In another exemplary embodiment, the peptide comprises at least a first isolated peptide of from 9 to about 11 amino acids in length, or at least a first nucleic acid segment that encodes the peptide; wherein the peptide consists essentially of the amino acid sequence of any one of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326.

Similarly, in another related embodiment, the peptide comprises at least a first isolated peptide of from 9 to about 10 or 11 or so amino acids in length, or at least a first nucleic acid segment that encodes the peptide; wherein the peptide consists of the amino acid sequence of any one of SEQ ID NO:13 to SEQ ID NO:20, SEQ ID NO:28 to SEQ ID NO:311, SEQ ID NO:313, SEQ ID NO:314, and SEQ ID NO:316 to SEQ ID NO:318, and particularly wherein the peptide consists of the amino acid sequence of any one of SEQ ID NO:34, SEQ ID NO:35, SEQ ID NO:49, SEQ ID NO:88, SEQ ID NO:144, SEQ ID NO:147, SEQ ID NO:185, SEQ ID NO:198, SEQ ID NO:199, and SEQ ID NO:282.

In addition to peptides and compositions that comprise a single peptide species, the invention also concerns compositions that comprise 2, 3, 4, or more peptide species and/or the polynucleotides that encode such peptides. Such pluralities of peptide and/or polynucleotide species are particularly desirable in the formulation of therapeutic agents that comprise pluralities of peptides having two or more different contiguous amino acid sequence as disclosed in the amino acid sequences of SEQ ID NO: 1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326, and/or a plurality of polynucleotides that encode such peptides. Irrespective of the source of the particular antigenic WT1 -derived peptide and polynucleotide compounds, the invention particularly contemplates the use of one, two, three or four distinct peptides, polynucleotides or derivatives thereof, up to and including a plurality of such compounds. This exemplifies the use of singular terminology throughout the entire application, wherein the terms “a” and “an” are used in the sense that they mean “at least one”, “at least a first”, “one or more” or “a plurality” of the referenced components or steps, except in instances wherein an upper limit is thereafter specifically stated or would be understood by one of ordinary skill in the art. The operable limits and parameters of combinations, as with the amounts of any single agent, will be known to those of ordinary skill in the art in light of the present disclosure.

The additional peptides in such compositions may all be of approximately the same size and/or approximately the same primary amino acid sequence, or alternatively, the peptides may differ considerably in length and/or primary amino acid sequence. Such compositions may further comprise one or more additional components, such as for example, a pharmaceutically acceptable excipient, buffer, or reagent as described in detail hereinbelow. Such compositions may also optionally further comprise at least a first immunostimulant or at least a first adjuvant as described herein. Such immunostimulants and adjuvants preferentially enhance a T-cell response in a human, and may preferably be selected from the group consisting of Montanide ISA50, Seppic Montanide ISA720, a cytokine, a microsphere, a dimethyl dioctadecyl ammonium bromide adjuvant, AS-1, AS-2, Ribi Adjuvant, QS21, saponin, microfluidized Syntex adjuvant, MV, ddMV, an immune stimulating complex and an inactivated toxin. As described in more detail hereinbelow, and particularly in Section 4, the compositions may be formulated for diagnostic or therapeutic uses, including their incorporation into one or more diagnostic or therapeutic kits for clinical packaging and/or commercial resale, with those formulations suitable for administration to a mammal, such as a human, with parenteral, intravenous, intraperitoneal, subcutaneous, intranasal, transdermal, and oral routes being particularly preferred.

The compositions may further optionally comprise one or more detection reagents, one or more additional diagnostic reagents, one or more control reagents, and/or one or more therapeutic reagents. In the case of diagnostic reagents, the compositions may further optionally comprise one or more detectable labels that may be used in both in vitro and/or in vivo diagnostic and therapeutic methodologies. In the case of therapeutic compositions and formulations, the compositions of the invention may also further optionally comprise one or more additional anti-cancer, anti-mesothelioma or otherwise therapeutically-beneficial components as may be required in particular circumstances, and such like.

In another aspect, the invention also provides methods for inhibiting the development of malignant mesothelioma in a human patient, comprising administering to a human patient a pharmaceutical composition comprising: (a) a WT1 peptide that comprises an immunogenic portion of a native WT1 or a variant thereof that differs in one or more substitutions, deletions, additions and/or insertions such that the ability of the variant to react with antigen-specific antibodies and/or T cell lines or clones is not substantially diminished; and (b) a physiologically acceptable carrier or excipient. Within certain embodiments, the patient is afflicted with malignant mesothelioma. In other embodiments, the composition is administered prophylactically to a patient considered at risk for the development of malignant mesothelioma. The WT1 peptide may, but need not, be present within a vaccine, which further comprises an immunostimulant, such as an adjuvant.

Within further aspects, methods are provided for inhibiting the development of malignant mesothelioma in a human patient, comprising administering to a human patient a pharmaceutical composition, comprising: (a) a polynucleotide encoding a WT1 peptide, wherein the peptide comprises an immunogenic portion of a native WT1 or a variant thereof that differs in one or more substitutions, deletions, additions and/or insertions such that the ability of the variant to react with antigen-specific antibodies and/or T cell lines or clones is not substantially diminished; and (b) a pharmaceutically acceptable carrier or excipient. Within certain embodiments, the patient is afflicted with malignant mesothelioma. In other embodiments, the composition is administered prophylactically to a patient considered at risk for the development of malignant mesothelioma. The WT1 polynucleotide may, but need not, be present within a vaccine, which further comprises an immunostimulant, such as an adjuvant.

Methods are further provided for inhibiting the development of malignant mesothelioma in a human patient, comprising administering to a human patient a pharmaceutical composition, comprising: (a) an antibody or antigen-binding fragment thereof that specifically binds to WT1,; and (b) a pharmaceutically acceptable carrier or excipient. Within certain embodiments, the patient is afflicted with malignant mesothelioma. In other embodiments, the composition is administered prophylactically to a patient considered at risk for the development of malignant mesothelioma.

Within further aspects, methods are provided for inhibiting the development of malignant mesothelioma in a human patient, comprising administering to a human patient a pharmaceutical composition, comprising: (a) a T cell that specifically reacts with WT1; and (b) a pharmaceutically acceptable carrier or excipient. Within certain embodiments, the patient is afflicted with malignant mesothelioma. In other embodiments, the composition is administered prophylactically to a patient considered at risk for the development of malignant mesothelioma.

Further methods for inhibiting the development of malignant mesothelioma in a human patient comprise administering to a human patient a pharmaceutical composition, comprising: (a) an antigen-presenting cell that expresses (i) a WT1 peptide that comprises an immunogenic portion of a native WT1 or a variant thereof that differs in one or more substitutions, deletions, additions and/or insertions such that the ability of the variant to react with antigen-specific antibodies and/or T cell lines or clones is not substantially diminished; and (b) a pharmaceutically acceptable carrier or excipient. Within certain embodiments, the patient is afflicted with malignant mesothelioma. In other embodiments, the composition is administered prophylactically to a patient considered at risk for the development of malignant mesothelioma. The antigen presenting cell may, but need not, be present within a vaccine, which further comprises an immunostimulant, such as an adjuvant.

Within other aspects, the present invention provides methods for inhibiting the development of malignant mesothelioma in a human patient, comprising administering to a human patient a preparation of stimulated and/or expanded T cells, wherein the T cells are stimulated and/or expanded by contact with a WT1 peptide, a polynucleotide encoding a WT1 peptide and/or an antigen-presenting cell that expresses a WT1 peptide. The T cells may be present, for example, within bone marrow, peripheral blood or a fraction of bone marrow or peripheral blood (e.g., obtained from a patient afflicted with malignant mesothelioma). The T cells may, but need not, be cloned prior to expansion.

Methods are further provided for inhibiting the development of malignant mesothelioma in a patient, comprising the steps of: (a) incubating CD4⁺ and/or CD8⁺ T cells isolated from a patient with one or more of: (i) a WT1 peptide; (ii) a polynucleotide encoding a WT1 peptide; or (iii) an antigen-presenting cell that expresses a WT1 peptide; such that the T cells proliferate; and (b) administering to the patient an effective amount of the proliferated T cells.

Further methods for inhibiting the development of malignant mesothelioma in a patient, comprising the steps of: (a) incubating CD4⁺ and/or CD8⁺ T cells isolated from a patient with one or more of: (i) a WT1 peptide; (ii) a polynucleotide encoding a WT1 peptide; or (iii) an antigen-presenting cell that expresses a WT1 peptide; such that the T cells proliferate; (b) cloning one or more cells that proliferated in the presence of WT1 peptide; and (c) administering to the patient an effective amount of the cloned T cells.

Within other aspects, the present invention provides method for determining the presence or absence of malignant mesothelioma in a patient, comprising the steps of: (a) incubating CD4⁺ and/or CD8⁺ T cells isolated from a patient with one or more of: (i) a WT1 peptide; (ii) a polynucleotide encoding a WT1 peptide; or (iii) an antigen-presenting cell that expresses a WT1 peptide; and (b) detecting the presence or absence of specific activation of the T cells. The step of detecting may comprise, for example, detecting the presence or absence of proliferation of the T cells or the generation of cytolytic activity.

The present invention further provides methods for determining the presence or absence of malignant mesothelioma in a patient, comprising the steps of: (a) incubating a biological sample obtained from a patient with one or more of: (i) a WT1 peptide; (ii) a polynucleotide encoding a WT1 peptide; or (iii) an antigen-presenting cell that expresses a WT1 peptide; wherein the incubation is performed under conditions and for a time sufficient to allow immunocomplexes to form; and (b) detecting immunocomplexes formed between the WT1 peptide and antibodies in the biological sample that specifically bind to the WT1, peptide. The step of detecting may comprise, for example, (a) incubating the immunocomplexes with a detection reagent that is capable of binding to the immunocomplexes, wherein the detection reagent comprises a reporter group, (b) removing unbound detection reagent, and (c) detecting the presence or absence of the reporter group.

Methods are further provided, within other aspects, for monitoring the effectiveness of an immunization or therapy for malignant mesothelioma in a patient, comprising the steps of: (a) incubating a first biological sample with one or more of: (i) a WT1 peptide; (ii) a polynucleotide encoding a WT1 peptide; or (iii) an antigen-presenting cell that expresses a WT1 peptide, wherein the first biological sample is obtained from a patient prior to a therapy or immunization, and wherein the incubation is performed under conditions and for a time sufficient to allow immunocomplexes to form; (b) detecting immunocomplexes formed between the WT1 peptide and antibodies in the biological sample that specifically bind to the WT1 peptide; (c) repeating steps (a) and (b) using a second biological sample obtained from the patient following therapy or immunization; and (d) comparing the number of immunocomplexes detected in the first and second biological samples. The step of detecting may comprise, for example, (a) incubating the immunocomplexes with a detection reagent that is capable of binding to the immunocomplexes, wherein the detection reagent comprises a reporter group, (b) removing unbound detection reagent, and (c) detecting the presence or absence of the reporter group.

Within further aspects, methods are provided for monitoring the effectiveness of an immunization or therapy for malignant mesothelioma in a patient, comprising the steps of: (a) incubating a first biological sample with one or more of: (i) a WT1 peptide; (ii) a WT1 polynucleotide encoding a WT1 peptide; or (iii) an antigen-presenting cell that expresses a WT1 peptide; wherein the biological sample comprises CD4⁺ and/or CD8⁺ T cells and is obtained from a patient prior to a therapy or immunization, and wherein the incubation is performed under conditions and for a time sufficient to allow specific activation, proliferation and/or lysis of T cells in the biological sample; (b) detecting an amount of activation, proliferation and/or lysis of the T cells; (c) repeating steps (a) and (b) using a second biological sample comprising CD4⁺ and/or CD8⁺ T cells, wherein the second biological sample is obtained from the same patient following therapy or immunization; and (d) comparing the amount of activation, proliferation and/or lysis of T cells in the first and second biological samples.

Throughout the methods of the invention, an “effective inhibitory amount” is an amount of at least a first WT1 compound effective to inhibit, and preferably to significantly inhibit, mesothelioma in an animal afflicted with such a disorder. The effective inhibitory amounts are thus also amounts effective to inhibit, and preferably to significantly inhibit, a biological activity of native WT1 polypeptide. More preferably, the effective inhibitory amounts are amounts of WT1 compounds effective to inhibit, and preferably to significantly inhibit, the biological activity of native WT1 polypeptide in a human having or suspected of having malignant pleural mesothelioma. Any degree of inhibition is sufficient to satisfy the invention, although those of ordinary skill in the art will understand the inhibition levels that are sufficient to indicate preferred in vitro and in vivo inhibition.

“Inhibition” requires a “reproducible,” i.e., consistently observed, inhibition in one or more of the foregoing parameters. A “significant inhibition” is a reproducible or consistently observed significant inhibition in one or more of the foregoing parameters, such as a reproducible inhibition of at least about 50%, about 55%, about 60%, about 65%, about 70%, about 75%, about 80%, or about 85% in comparison to control levels, i.e., in the absence of the WT1 therapeutic composition. Although not required to practice the invention, inhibition levels of at least about 90%, about 92%, about 94%, about 96%, or even about 98% or higher are by no means excluded.

Execution of one or more of the therapeutic methods disclosed herein gives rise to effective therapies for preventing or treating malignant mesothelioma. These methods, which typically comprise providing to an animal or patient having, suspected of having, or at risk for developing malignant mesothelioma, an amount of at least a first WT1 peptide, antibody, antigen presenting cell, T cell, antigen binding fragment, or polynucleotide effective to inhibit malignant mesothelioma within cells of the animal or patient, thereby preventing or treating malignant mesothelioma.

The foregoing “prophylactically and therapeutically effective amounts” are thus encompassed within the terms “biologically effective amounts” and “effective inhibitory amounts” of WT1 peptide, antibody, antigen presenting cell, T cell, antigen binding fragment, or polynucleotide compositions. All such “effective amounts” are amounts of the disclosed WT1 compounds effective to produce some, and preferably some significant, benefit upon administration to an animal or patient. The benefits include reducing symptoms, severity and/or duration, as well as lessening the chance of transmission and other veterinary and clinical benefits.

The routes of administration that may be used in the present invention are virtually limitless, so long as an effective amount of at least a first WT1 peptide, antibody, antigen presenting cell, T cell, antigen binding fragment, or polynucleotide composition can be provided thereby. Exemplary means for therapeutic delivery of the disclosed compositions, including e.g., ingestion, inhalation, transdermal, parenteral administration, intranasal administration, subcutaneous injection, intravenous injection, continuous infusion, and the like are discussed in more detail hereinbelow.

All such compositions and methods of the invention may be combined for use with one or more other anti-cancer agents, such as at least a second, third, fourth or fifth, anti-mesothelioma agent or at least a first, second, third or fourth anti-cancer therapeutic agent. A plurality of distinct anti-cancer or anti-mesothelioma therapeutic agents may be administered to an animal or patient, up to and including the dose limiting toxicity of the combination. The invention can thus be used to form synergistic combinations with other therapies and/or known agents, particularly those methods and agents that previously failed to achieve maximal effectiveness in vivo, perhaps due to dose-limiting toxicity and/or resistance.

In such combination therapies, the at least a first WT1 peptide, antibody, antigen presenting cell, T cell, antigen binding fragment, or polynucleotide, and at least a second anti-mesothelioma or anti-cancer therapeutic agent may be administered to the animal or patient substantially simultaneously, such as from a single pharmaceutical formulation or two distinct pharmaceutical formulations. Alternatively, the at least a first WT1 peptide, antibody, antigen presenting cell, T cell, antigen binding fragment, or polynucleotide, and at least a second anti-mesothelioma or anti-cancer therapeutic agent may be administered to the animal or patient sequentially, such as on alternate days.

In further embodiments, the invention provides a range of therapeutic kits. Certain kits comprise a therapeutically effective amount of at least a first WT1 peptide, antibody, antigen presenting cell, T cell, antigen binding fragment, or polynucleotide composition and instructions for administering the composition to an animal or subject having or at risk for developing mesothelioma, and in particular, malignant pleural mesothelioma. Such kits may be combined with effective amounts of at least one diagnostic agent that detects a WT1 polypeptide or antibody, or at least one diagnostic agent that detects a mesothelioma cell; or with a therapeutically effective amount of at least one other anti-cancer, anti-mesothelioma or anti-WT1 polypeptide therapeutic agent.

Certain other therapeutic kits and uses of the compositions disclosed herein, may comprise an effective amount of at least a first WT1 peptide, antibody, antigen presenting cell, T cell, antigen binding fragment, or polynucleotide and an effective amount of at least one diagnostic agent that detects detects a mesothelioma cell; or an effective amount of at least one, two, three, four or any number of other anti-cancer, anti-mesothelioma or anti-WT1 polypeptide therapeutic agents. Instructions may also be combined with these kits. Other biological agents or components may be included, such as those for making and using the drugs.

Exemplary diagnostic agents include molecular biological agents that detect at least a first WT1-encoding nucleic acid; at least a first WT1 peptide or polypeptide, at least a first antibody that detects at least a first WT1 protein or peptide; and at least a first WT1 protein or peptide that detects at least a first antibody that binds to a WT1 protein or peptide. The range of additional therapeutic agents will be known those of ordinary skill in the art in light of the present disclosure, as exemplified by those described herein.

In such kits, the diagnostic agents are preferably disposed within a distinct container of the kit. The combined therapeutic agents, however, may be combined within a single container of the kit, i.e., in the same composition as the WT1 composition, such as in a “cocktail” or admixture. They may alternatively be maintained separately from the WT1 compound, in a distinct container.

The invention thus provides combination therapeutics comprising, in any pharmaceutically acceptable form, a therapeutically effective amount of a WT1 compound in combination with a therapeutically effective amount of at least a second anti-WT1 anti-mesothelioma or anti-cancer therapeutic agent. Also provided are compositions for use in the manufacture of a medicament or medicinal cocktail, that comprise, in any pharmaceutically acceptable form, a therapeutically effective amount of at least a first WT1 composition. Moreover, the invention provides compositions for use in the manufacture of a medicament or medicinal cocktail that comprise, in any pharmaceutically acceptable form, a first WT1 composition and a plurality of distinct anti-WT1, anti-mesothelioma or anti-cancer therapeutic agents. Combined uses and medicaments in which a WT1 compound is one component of a therapeutic approach are also encompassed within the present invention.

These and other aspects of the present invention will become apparent upon reference to the following detailed description and attached drawings. All references disclosed herein are hereby incorporated by reference in their entirety as if each was incorporated individually.

### 3. BRIEF DESCRIPTION OF THE DRAWINGS

The invention may be understood by reference to the following description taken in conjunction with the accompanying drawings, in which like reference numerals identify like elements, and in which:

FIG. 1 depicts a comparison of the mouse (MO) (SEQ ID NO:320) and human (HU) (SEQ ID NO:319) WT1 peptide sequences;

FIG. 2 depicts a histogram presenting the results of an ELISA assay to detect WT1-specific antibodies in malignant mesothelioma patients. WT180 and WTC19, as indicated, represent positive controls. D44 represents normal control serum, and the remaining samples were serum samples obtained from human patients afflicted with malignant mesothelioma;

FIG. 3A, FIG. 3B and FIG. 3C depict graphs illustrating the stimulation of proliferative T cell responses in mice immunized with representative WT1 peptides. Thymidine incorporation assays were performed using one T cell line and two different clones, as indicated, and results were expressed as cpm. Controls indicated on the X-axis were no antigen (No Ag) and B6/media; antigens used were p6-22 human (p 1), p117-139 (p2) or p244-262 human (p3).

FIG. 4A and FIG. 4B show histograms illustrating the stimulation of proliferative T cell responses in mice immunized with representative WT1 peptides. Three weeks after the third immunization, spleen cells of mice that had been inoculated with Vaccine A or Vaccine B were cultured with medium alone (medium) or spleen cells and medium (B6/no antigen), B6 spleen cells pulsed with the peptides p6-22 (p6), p117-139 (p117), p244-262 (p244) (Vaccine A; FIG. 4A) or p287-301 (p287), p299-313 (p299), p421-435 (p421) (Vaccine B; FIG. 4B) and spleen cells pulsed with an irrelevant control peptide (irrelevant peptide) at 25 μg/ml and were assayed after 96 hr for proliferation by (3 H) thymidine incorporation. Bars represent the stimulation index (SI), which is calculated as the mean of the experimental wells divided by the mean of the control (B6 spleen cells with no antigen);

FIG. 5A, FIG. 5B, FIG. 5C, and FIG. 5D are histograms illustrating the generation of proliferative T-cell lines and clones specific for p117-139 and p6-22. Following in vivo immunization, the initial three in vitro stimulations (IVS) were carried out using all three peptides of Vaccine A or B, respectively. Subsequent IVS were carried out as single peptide stimulations using only the two relevant peptides pl 17-139 and p6-22. Clones were derived from both the p6-22 and p117-139 specific T cell lines, as indicated. T cells were cultured with medium alone (medium) or spleen cells and medium (B6/no antigen), B6 spleen cells pulsed with the peptides p6-22 (p6), p117-139 (p117) or an irrelevant control peptide (irrelevant peptide) at 25 μg/ml and were assayed after 96 hr for proliferation by (³H) thymidine incorporation. Bars represent the stimulation index (SI), which is calculated as the mean of the experimental wells divided by the mean of the control (B6 spleen cells with no antigen);

FIG. 6A and FIG. 6B are graphs illustrating the elicitation of WT1 peptide-specific CTL in mice immunized with WT1 peptides.

FIG. 6A illustrates the lysis of target cells by allogeneic cell lines and FIG.

6B shows the lysis of peptide coated cell lines. In each case, the % lysis (as determined by standard chromium release assays) is shown at three indicated effector:target ratios. Results are provided for lymphoma cells (LSTRA and E10), as well as E10+p235-243 (E10+P235). E10 cells are also referred to herein as EL-4 cells;

FIG. 7A, FIG. 7B, FIG. 7C, and FIG. 7D are graphs illustrating the elicitation of WT1 specific CTL, which kill WT1 positive tumor cell lines but do not kill WT1 negative cell lines, following vaccination of B6 mice with WT1 peptide P117.

FIG. 7A illustrates that T-cells of non-immunized B6 mice do not kill WT1 positive tumor cell lines.

FIG. 7B illustrates the lysis of the target cells by allogeneic cell lines.

FIG. 7C and FIG. 7D demonstrate the lysis of WT1 positive tumor cell lines, as compared to WT1 negative cell lines in two different studies. In addition, FIG. 7C and FIG. 7D show the lysis of peptide-coated cell lines (WT1 negative cell line E10 coated with the relevant WT1 peptide P117). In each case, the % lysis (as determined by standard chromium release assays) is shown at three indicated effector:target ratios. Results are provided for lymphoma cells (El0), prostate cancer cells (TRAMP-C), a transformed fibroblast cell line (BLK-SV40), as well as E10+p117;

FIG. 8A and FIG. 8B are histograms illustrating the ability of representative peptide P117-139 specific CTL to lyse WT1 positive tumor cells. Three weeks after the third immunization, spleen cells of mice that had been inoculated with the peptides p235-243 or p117-139 were stimulated in vitro with the relevant peptide and tested for ability to lyse targets incubated with WT1 peptides as well as WT1 positive and negative tumor cells. The bars represent the mean % specific lysis in chromium release assays performed in triplicate with an E:T ratio of 25:1.

FIG. 8A shows the cytotoxic activity of the p235-243 specific T cell line against the WT1 negative cell line EL-4 (EL-4, WT1 negative); EL-4 pulsed with the relevant (used for immunization as well as for restimulation) peptide p235-243 (EL-4+p235); EL-4 pulsed with the irrelevant peptides p117-139 (EL-4+p117), p126-134 (EL-4+p126) or p130-138 (EL-4+p130) and the WT1 positive tumor cells BLK-SV40 (BLK-SV40, WT1 positive) and TRAMP-C (TRAMP-C, WT1 positive), as indicated.

FIG. 8B shows cytotoxic activity of the p117-139 specific T cell line against EL-4; EL-4 pulsed with the relevant peptide P117-139 (EL-4+p117) and EL-4 pulsed with the irrelevant peptides p123-131 (EL-4+p123), or p128-136 (EL-4+p128); BLK-SV40 and TRAMP-C, as indicated;

FIG. 9A and FIG. 9B are histograms illustrating the specificity of lysis of WT1 positive tumor cells, as demonstrated by cold target inhibition. The bars represent the mean % specific lysis in chromium release assays performed in triplicate with an E:T ratio of 25:1.

FIG. 9A shows the cytotoxic activity of the pl 17-139 specific T cell line against the WT1 negative cell line EL-4 (EL-4, WT1 negative); the WT1 positive tumor cell line TRAMP-C (TRAMP-C, WT1 positive); TRAMP-C cells incubated with a ten-fold excess (compared to the hot target) of EL-4 cells pulsed with the relevant peptide pl 17-139 (TRAMP-C +p117 cold target) without ⁵¹Cr labeling and TRAMP-C cells incubated with EL-4 pulsed with an irrelevant peptide without ⁵Cr labeling (TRAMP-C+irrelevant cold target), as indicated.

FIG. 9B shows the cytotoxic activity of the pl 17-139 specific T cell line against the WT1 negative cell line EL-4 (EL-4, WT1 negative); the WT1 positive tumor cell line BLK-SV40 (BLK-SV40, WT1 positive); BLK-SV40 cells incubated with the relevant cold target (BLK-SV40+p117 cold target) and BLK-SV40 cells incubated with the irrelevant cold target (BLK-SV40+irrelevant cold target), as indicated;

FIG. 10A, FIG. 10B, and FIG. 10C are histograms depicting an evaluation of the nonapeptide CTL epitope within p117-139. The p117-139 tumor specific CTL line was tested against peptides within aa117-139 containing or lacking an appropriate H-2^{b} class I binding motif and following restimulation with p126-134 or p130-138. The bars represent the mean % specific lysis in chromium release assays performed in triplicate with an E:T ratio of 25:1.

FIG. 10A shows the cytotoxic activity of the p117-139 specific T cell line against the WT1 negative cell line EL-4 (EL-4, WT1 negative) and EL-4 cells pulsed with the peptides p117-139 (EL-4 +p117), p119-127 (EL-4 +p119), p120-128 (EL-4 +p120), p123-131 (EL-4+p123), p126-134 (EL-4+p126), p128-136 (EL-4+p128), and p130-138 (EL-4+p130).

FIG. 10B shows the cytotoxic activity of the CTL line after restimulation with p126-134 against the WT1 negative cell line EL-4, EL-4 cells pulsed with p117-139 (EL-4+p117), p126-134 (EL-4+p126) and the WT1 positive tumor cell line TRAMP-C; and

FIG. 10C shows the cytotoxic activity of the CTL line after restimulation with p130-138 against EL-4, EL-4 cells pulsed with p117-139 (EL-4+p117), p130-138 (EL-4+p130) and the WT1 positive tumor cell line TRAMP-C.

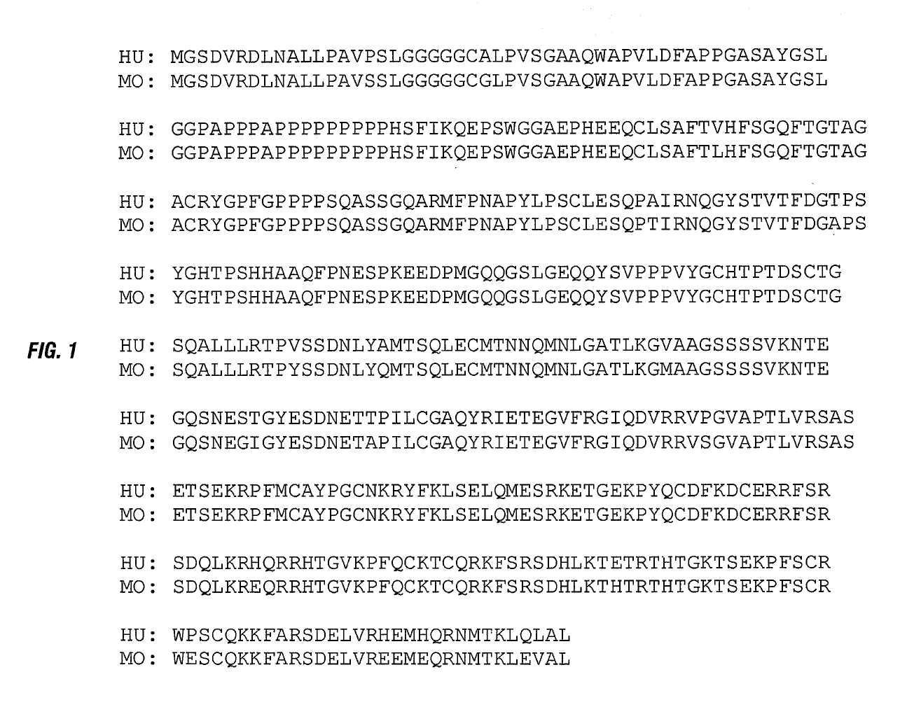

### 4. DESCRIPTION OF ILLUSTRATIVE EMBODIMENTS

In order that the invention herein described may be more fully understood, the following description of various illustrative embodiments is set forth.

The present invention is generally directed to compositions and methods for the immunotherapy and diagnosis of WT1 -associated diseases, such as malignant mesothelioma. In particular WT1 expression, and immune responses to WT1 (e.g., the presence of WT1 specific antibodies in patient sera), may be used as markers to identify patients with malignant mesothelioma and other WT1 associated malignancies (such as leukemia (e.g., acute myeloid leukemia (AML), chronic myeloid leukemia (CML), acute lymphocytic leukemia (ALL) and childhood ALL), Myelodysplastic syndromes, myeloproliferative syndromes, prostate cancer, lung cancer, breast cancer, thyroid cancer, gastrointestinal cancer, kidney cancer, liver cancer, ovarian cancer, testicular cancer and melanoma). Such diagnostic methods (e.g., in high throughput assay format) may be used for early diagnosis of cancer, and permit screening of healthy individuals who have or might have been exposed to asbestos. Patients found to be afflicted with such malignancies may benefit from the WT1-based vaccine or T-cell therapeutic methods provided herein.

The compositions described herein generally comprise WT1 peptides, WT1 polynucleotides, antigen-presenting cells (APC; e.g., dendritic cells) that express a WT1, peptide, agents such as antibodies that specifically bind to a WT1 polypeptides and WT1-derived peptides; and/or immune system cells (e.g., T cells) specific for WT1. WT1 peptides of the present invention generally comprise at least a portion of a Wilms' tumor gene product (WT1) or a variant thereof. Nucleic acid sequences of the subject invention generally comprise a DNA, PNA, or RNA sequence that encodes all or a portion of such a peptide, or that is complementary to such a sequence. Antibodies are generally immune system proteins, or antigen-binding fragments thereof, that are capable of binding to a portion of a WT1 peptide. T cells that may be employed within such compositions are generally cells (e.g., CD4⁺ and/or CD8⁺) that are specific for a WT1 peptide. Certain methods described herein further employ one or more antigen-presenting cells that express at least a first WT1 peptide or polypeptide as provided herein.

4.1 WT1 Peptides

Within the context of the present invention, exemplary preferred WT1-derived antigenic peptides include those peptides of from 9 to about 100 amino acids in length, that comprises at least a first epitope, antigenic fragment, antibody binding site, or an immunogenic sequence that is selected from the group consisting of SEQ ID NO: 1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ IDNO:326.

The WT1-derived peptides may be of any intermediate length provided that it comprises at least a first immunogenic portion or epitope, or antibody binding site, of a native WT1 polypeptide or a variant thereof, and particularly those peptide sequences disclosed in any one of SEQ ID NO:1 to SEQ ID NO:4, SEQ ID NO: 13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326. In other words, a WT1 peptide may be an oligopeptide (i.e., those consisting of a relatively small number of amino acid residues, such as 9 to about 12 or 13 or so amino acid residues), larger oligopeptides (i.e., those consisting of a relatively larger number of amino acid residues, such as for example, about 14 to about 20 or so amino acid residues), still larger peptides (i.e., those consisting of a relatively larger number of amino acid residues, such as for example, about 21 to about 40 or so amino acid residues), and so forth, up to and including those peptides that consist of a significantly larger number of amino acid residues, such as for example, about 5 to about 90 or 100 or so amino acid residues, as well as all peptides of intermediate sizes.

Within certain embodiments, the use of WT1 peptides that contain a small number of consecutive amino acid residues of a native WT1 peptide is preferred. Such peptides are preferred for certain uses in which the generation of a T cell response is desired. For example, such a WT1 peptide preferably contain at least 9, or at least about 10, 11, 12, 13, 14, or 15 or more consecutive amino acid residues of the native WT1 polypeptide. Nonameric peptides (9-mers, or those comprising at least nine consecutive amino acid residues of a native WT1 polypeptide) are particularly contemplated to be useful in the methods disclosed herein. Additional sequences derived from the native Protein A and/or heterologous sequences may be present within any WT1 peptide, and such sequences may (but need not) possess further immunogenic or antigenic properties. Peptides as provided herein may further be associated (covalently or noncovalently) with other peptide or non-peptide compounds.

An “immunogenic portion,” as used herein is a portion of a peptide that is recognized (i.e., specifically bound) by a B-cell and/or T-cell surface antigen receptor. Certain preferred immunogenic portions bind to an MHC class I or class II molecule. As used herein, an immunogenic portion is said to “bind to” an MHC class I or class II molecule if such binding is detectable using any assay known in the art. For example, the ability of a peptide to bind to MHC class I may be evaluated indirectly by monitoring the ability to promote incorporation of ¹²⁵I-labeled β2-microglobulin (β2 m) into MHC class I/β2 m/peptide heterotrimeric complexes (Parker et al., 1994). Alternatively, functional peptide competition assays that are known in the art may be employed. Certain immunogenic portions have one or more of the sequences recited within one or more of Tables 2-14.

Exemplary immunogenic peptides of the present invention include, but are not limited to, those disclosed in the Examples and illustrated in Table 2 through Table 49, and particularly, peptides that comprise at least a first amino acid sequence as defined in any one of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326.

Illustrative WT1-derived peptide compositions include, but are not limited to, those that comprise at least a first amino acid sequence selected from the group consisting of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, and SEQ ID NO:316 to SEQ ID NO:318, and particularly such sequences as disclosed in any one of the following: RDLNALLPAVPSLGGGG (human WT1 residues 6-22; SEQ ID NO:1), PSQASSGQARMFPNAPYLPSCLE (human and mouse WT1 residues 117-139; SEQ ID NO:2 and SEQ ID NO:3, respectively), GATLKGVAAGSSSSVKWTE (human WT1 residues 244-262; SEQ ID NO:4), GATLKGVAA (human WT1 residues 244-252; SEQ ID NO:88), CMTWNQMNL (human and mouse WT1 residues 235-243; SEQ ID NO:49 and SEQ ID NO:258, respectively), SCLESQPTI (mouse WT1 residues 136-144; SEQ ID NO:296), SCLESQPAI (human WT1 residues 136-144; SEQ ID NO:198), NLYQMTSQL (human and mouse WT1 residues 225-233; SEQ ID NO:147 and SEQ ID NO:284, respectively); ALLPAVSSL (mouse WT1 residues 10-18; SEQ ID NO:255); or RMFPNAPYL (human and mouse WT1 residues 126-134; SEQ ID NO:185 and SEQ ID NO:293, respectively).

Further immunogenic fragments and peptides are provided herein, and others may generally be identified using well-known techniques (Paul, 1993). Representative techniques for identifying immunogenic peptides, epitopes, and antibody binding motifs include, for example, screening peptides for the ability to react with antigen-specific antisera and/or T-cell lines or clones. An immunogenic portion of a native WT1 polypeptide is a portion that reacts with such antisera and/or T-cells at a level that is not substantially less than the reactivity of the full length WT1 (e.g., in an ELISA and/or T-cell reactivity assay). In other words, an immunogenic portion may react within such assays at a level that is similar to or greater than the reactivity of the full-length polypeptide. Such screens may generally be performed using methods well known to those of ordinary skill in the art (Harlow and Lane, 1988).

Alternatively, immunogenic portions may be identified using computer analysis, such as the Tsites program (Rothbard and Taylor, 1988; Deavin et al, 1996), which searches for peptide motifs that have the potential to elicit Th responses. CTL peptides with motifs appropriate for binding to murine and human class I or class II MHC may be identified according to BIMAS (Parker et al., 1994) and other HLA peptide binding prediction analyses. To confirm immunogenicity, a peptide may be tested using an HLA A2 transgenic mouse model and/or an in vitro stimulation assay using dendritic cells, fibroblasts or peripheral blood cells.

As noted above, the peptides of the present invention may comprise one or more variants of the amino acid sequences as disclosed herein. A peptide “variant,” as used herein, is a peptide that differs from a particular primary amino acid sequence in one or more substitutions, deletions, additions and/or insertions, such that the immunogenicity of the peptide is substantially retained (i.e., the ability of the variant to react with antigen-specific antisera and/or T-cell lines or clones is not substantially diminished relative to the native peptide). In other words, the ability of a variant to react with antigen-specific antisera and/or T-cell lines or clones may be enhanced or unchanged, relative to the peptide from which the variant was derived.

Preferably, the biological activity of a peptide variant will not be diminished by more than 1%, and preferably still will not be diminished by more than 2%, relative to the biological activity of the unmodified peptide. More preferably, the biological activity of a peptide variant will not be diminished by more than 3%, and more preferably still will not be diminished by more than 4%, 5%, 6%, 7%, 8%, or 9%, relative to the biological activity of the unmodified peptide. More preferably still, the biological activity of a peptide variant will not be diminished by more than 10%, and more preferably still, will not be diminished by more than 11%, 12%, 13%, 14%, 15%, 16%, 17%, 18%, 19%, or 20% relative to the biological activity of the corresponding unmodified peptide.

Based upon % sequence homology, preferred peptide variant of the present invention include those peptides that are from 9 to about 100 amino acids in length, and that comprise at least a first sequence region that is at least 75% identical to at least one of the amino acid sequences dislosed in any one of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326, and more preferably those that comprise at least a first sequence region that is at least 80% identical to at least one of the amino acid sequences dislosed in any one of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326. More preferably, based upon % sequence homology, preferred peptide variants of the present invention are those peptides that comprise at least a first sequence region that is at least 85% identical to at least one of the amino acid sequences dislosed in any one of SEQ ID NO: 1 to SEQ ID NO:4, SEQ ID NO: 13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326, and more preferably those that comprise at least a first sequence region that is at least 90% identical to at least one of the amino acid sequences dislosed in any one of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326. Particularly preferred peptide variants of the present invention are those peptides that comprise at least a first sequence region that is at least 91%, 92%, 93%, 94%, or 95% identical to at least one of the amino acid sequences dislosed in any one of SEQ ID NO:1 to SEQ ID NO:4, SEQ ID NO: 13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326, with those peptides that comprise at least a first sequence region that is at least 96%, 97%, 98%, or 99% identical to at least one of the amino acid sequences dislosed in any one of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326.

Such peptide variants may typically be prepared by modifying one of the peptide sequences disclosed herein, and particularly by modifying the primary amino acid sequence of one or more of the nonameric peptide epitopes disclosed in any one of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326. These biological functional equivalent peptides may encompass primary amino acid sequences that differ from the original peptide sequences disclosed in any one of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326 by one or more conservative amino acid substitutions.

It has been found, within the context of the present invention, that a relatively small number of conservative or neutral substitutions (e.g., 1 or 2) may be made within the sequence of the nonameric peptide epitopes disclosed herein, without substantially altering the biological activity of the peptide. In some cases, the substitution of one or more amino acids in a particular peptide may in fact serve to enhance or otherwise improve the ability of the peptide to elicit an immune or T-cell response in an animal that has been provided with a composition that comprises the modified peptide, or a polynucleotide that encodes the peptide. Suitable substitutions may generally be identified by using computer programs, as described hereinbelow, and the effect of such substitutions may be confirmed based on the reactivity of the modified peptide with antisera and/or T-cells as described herein. Accordingly, within certain preferred embodiments, a WT1 peptide for use in the disclosed diagnostic and therapeutic methods may comprise a primary amino acid sequence in which one or more amino acid residues are substituted by one or more replacement amino acids, such that the ability of the modified peptide to react with antigen-specific antisera and/or T-cell lines or clones is not significantly less than that for the unmodified peptide. Exemplary such substitutions may preferably be located within one or more MHC binding sites on the peptide.

As described above, preferred peptide variants are those that contain one or more conservative substitutions. A “conservative substitution” is one in which an amino acid is substituted for another amino acid that has similar properties, such that one skilled in the art of peptide chemistry would expect the secondary structure and hydropathic nature of the peptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Examples of amino acid substitutions that represent a conservative change include: (1) replacement of one or more Ala, Pro, Gly, Glu, Asp, Gln, Asn, Ser, or Thr; residues with one or more residues from the same group; (2) replacement of one or more Cys, Ser, Tyr, or Thr residues with one or more residues from the same group; (3) replacement of one or more Val, Ile, Leu, Met, Ala, or Phe residues with one or more residues from the same group; (4) replacement of one or more Lys, Arg, or His residues with one or more residues from the same group; and (5) replacement of one or more Phe, Tyr, Trp, or His residues with one or more residues from the same group.

A variant may also, or alternatively, contain nonconservative changes, for example, by substituting one of the amino acid residues from group (1) with an amino acid residue from group (2), group (3), group (4), or group (5). Variants may also (or alternatively) be modified by, for example, the deletion or addition of amino acids that have minimal influence on the immunogenicity, secondary structure and hydropathic nature of the peptide.

4.2 Biological Functional Equivalents

Modification and changes may be made in the structure of the polynucleotides and peptides of the present invention and still obtain a functional molecule that encodes a peptide with desirable characteristics, or still obtain a genetic construct with the desirable expression specificity and/or properties. As it is often desirable to introduce one or more mutations into a specific polynucleotide sequence, various means of introducing mutations into a polynucleotide or peptide sequence known to those of skill in the art may be employed for the preparation of heterologous sequences that may be introduced into the selected cell or animal species. In certain circumstances, the resulting encoded peptide sequence is altered by this mutation, or in other cases, the sequence of the peptide is unchanged by one or more mutations in the encoding polynucleotide. In other circumstances, one or more changes are introduced into the promoter and/or enhancer regions of the polynucleotide constructs to alter the activity, or specificity of the expression elements and thus alter the expression of the heterologous therapeutic nucleic acid segment operably positioned under the control of the elements.

When it is desirable to alter the amino acid sequence of one or more of the heterologous peptides encoded by the expression construct to create an equivalent, or even an improved, second-generation molecules, the amino acid changes may be achieved by changing one or more of the codons of the encoding DNA sequence, according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions, or corresponding DNA sequences which encode said peptides without appreciable loss of their biological utility or activity.

TABLE 1

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In making such changes, the hydropathic index of amino acids may be considered. The importance of the hydropathic amino acid index in conferring interactive biologic function on a protein is generally understood in the art (Kyte and Doolittle, 1982, incorporate herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those that are within ±1 are particularly preferred, and those within ±0.5 are even more particularly preferred. It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take several of the foregoing characteristics into consideration are well known to those of skill in the art and include: arginine and lysine; glutamate and aspartate; serine and threonine; glutamine and asparagine; and valine, leucine and isoleucine.

The peptides and peptide variants of the present invention may be conjugated to a signal (or leader) sequence at the N-terminal end of the peptide, which co-translationally or post-translationally directs transfer of the peptide. The peptides may also, or alternatively, be conjugated to one or more linker sequences for ease of synthesis, purification or identification of the peptide (e.g., poly-His), or to enhance binding of the peptide to a solid support. For example, the peptides may be conjugated to an immunoglobulin Fc region.

The peptides and peptide variants of the present invention may be isolated and purified from native sources, such as for example, by isolating all or part of the primary amino acid sequence from a native WT1 peptide, or alternatively, may be chemically synthesized in whole or in part using any of a variety of well-known peptide synthesis techniques. For example, peptides having less than about 100 amino acids, preferably less than about 90 or 80 amino acids, and more preferably less than about 70, less than about 60, or less than about 50, about 40, about 30, or about 20 amino acids, may be synthesized using any of the commercially available solid-phase techniques, such as the Merrifield solid-phase synthesis method, where amino acids are sequentially added to a growing amino acid chain (Merrifield, 1963). Equipment for automated synthesis of peptides is commercially available from suppliers such as Applied BioSystems, Inc. (Foster City, Calif.), and may be operated according to the manufacturer's instructions.

The peptides and peptide variants as described herein may also be readily prepared from recombinant WT1 peptides, or may be prepared by translation of a polynucleotide sequence that encodes such a peptide. In general, any of a variety of expression vectors known to those of ordinary skill in the art may be employed to express recombinant peptides. Expression may be achieved in any appropriate host cell that has been transformed or transfected with an expression vector containing a nucleic acid molecule that encodes the peptide. Suitable host cells include prokaryotes, yeast and higher eukaryotic cells. Preferably, the host cells employed are E. coli, yeast or a mammalian cell line such as COS or CHO.

In general, peptides and polynucleotides as described herein are isolated. An “isolated” peptide or polynucleotide is one that is removed from its original environment. For example, a naturally occurring peptide or polypeptide is isolated if it is separated from some or all of the coexisting materials in the natural system. Preferably, such peptides are at least about 80% or 85% pure, more preferably at least about 90% or 95% pure and most preferably at least about 96%, 97%, 98%, or 99% pure. A polynucleotide is considered to be isolated if, for example, it is cloned into a vector that is not a part of the natural environment.

Within further aspects, the present invention provides mimetics of WT1 peptides. Such mimetics may comprise amino acids linked to one or more amino acid mimetics (i.e., one or more amino acids within the WT1 protein may be replaced by an amino acid mimetic) or may be entirely nonpeptide mimetics. An amino acid mimetic is a compound that is conformationally similar to an amino acid such that it can be substituted for an amino acid within a WT1 peptide without substantially diminishing the ability to react with antigen-specific antisera and/or T cell lines or clones. A nonpeptide mimetic is a compound that does not contain amino acids, and that has an overall conformation that is similar to a WT1 peptide such that the ability of the mimetic to react with WT1 -specific antisera and/or T cell lines or clones is not substantially diminished relative to the ability of a WT1 peptide. Such mimetics may be designed based on standard techniques (e.g., nuclear magnetic resonance and computational techniques) that evaluate the three dimensional structure of a peptide sequence. Mimetics may be designed where one or more of the side chain functionalities of the WT1 peptide are replaced by groups that do not necessarily have the same size or volume, but have similar chemical and/or physical properties which produce similar biological responses. It should be understood that, within embodiments described herein, a mimetic may be substituted for a WT1 peptide.

Within other illustrative embodiments, a polypeptide may be a fusion polypeptide that comprises multiple polypeptides as described herein, or that comprises at least one polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the polypeptide or to enable the polypeptide to be targeted to desired intracellular compartments. Still further fusion partners include affinity tags, which facilitate purification of the polypeptide.

Fusion polypeptides may generally be prepared using standard techniques, including chemical conjugation. Preferably, a fusion polypeptide is expressed as a recombinant polypeptide, allowing the production of increased levels, relative to a non-fused polypeptide, in an expression system. Briefly, DNA sequences encoding the polypeptide components may be assembled separately, and ligated into an appropriate expression vector. The 3′-end of the DNA sequence encoding one polypeptide component is ligated, with or without a peptide linker, to the 5′-end of a DNA sequence encoding the second polypeptide component so that the reading frames of the sequences are in phase. This permits translation into a single fusion polypeptide that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion polypeptide using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., 1985; Murphy et al., 1986; U.S. Pat. No. 4,935,233 and U.S. Pat. No. 4,751,180. The linker sequence may generally be from 1 to about 10, about 20, about 30, about 40, or about 50 or so amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

The fusion polypeptide can comprise a polypeptide as described herein together with an unrelated immunogenic protein, such as an immunogenic protein capable of eliciting a recall response. Examples of such proteins include tetanus, tuberculosis and hepatitis proteins (see, for example, Stoute et al., 1997).

In one preferred embodiment, the immunological fusion partner is derived from a Mycobacterium sp., such as a Mycobacterium tuberculosis-derived Ra12 fragment. Ra12 compositions and methods for their use in enhancing the expression and/or immunogenicity of heterologous polynucleotide/polypeptide sequences is described in U.S. patent application 60/158,585, the disclosure of which is incorporated herein by reference in its entirety. Briefly, Ra12 refers to a polynucleotide region that is a subsequence of a Mycobacterium tuberculosis MTB32A nucleic acid. MTB32A is a serine protease of 32 kDa molecular weight encoded by a gene in virulent and avirulent strains of M tuberculosis. The nucleotide sequence and amino acid sequence of MTB32A have been described (see for example, U.S. patent application 60/158,585; and Skeiky et al., 1999, each incorporated herein by reference). C-terminal fragments of the MTB32A coding sequence express at high levels and remain as soluble polypeptides throughout the purification process. Moreover, Ra12 may enhance the immunogenicity of heterologous immunogenic polypeptides with which it is fused. One preferred Ra12 fusion polypeptide comprises a 14-kDa C-terminal fragment corresponding to amino acid residues 192 to 323 of MTB32A. Other preferred Ra12 polynucleotides generally comprise at least about 15 consecutive nucleotides, at least about 30 nucleotides, at least about 60 nucleotides, at least about 100 nucleotides, at least about 200 nucleotides, or at least about 300 nucleotides that encode a portion of a Ra12 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants preferably exhibit at least about 70% identity, more preferably at least about 80% identity and most preferably at least about 90% identity to a polynucleotide sequence that encodes a native Ra12 polypeptide or a portion thereof.

Within other preferred embodiments, an immunological fusion partner is derived from Protein D, a surface protein of the gram-negative bacterium Haemophilus influenza B (Intl. Pat. Appl. Publ. No. WO 91/18926). Preferably, a Protein D derivative comprises approximately the first third of the protein (e.g., the first N-terminal 100-110 amino acids), and a Protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. Coli (thus functioning as an expression enhancer). The lipid tail ensures optimal presentation of the antigen to antigen-presenting cells. Other fusion partners include the non-structural protein from influenzae virus, NSI (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described. Within a preferred embodiment, a repeat portion of LYTA may be incorporated into a fusion polypeptide. A repeat portion is found in the C-terminal region starting at residue 178. A particularly preferred repeat portion incorporates residues 188-305.

Yet another illustrative embodiment involves fusion polypeptides, and the polynucleotides encoding them, wherein the fusion partner comprises a targeting signal capable of directing a polypeptide to the endosomal/lysosomal compartment, as described in U.S. Pat. No. 5,633,234. An immunogenic polypeptide of the invention, when fused with this targeting signal, will associate more efficiently with MHC class II molecules and thereby provide enhanced in vivo stimulation of CD4⁺ T-cells specific for the polypeptide.

4.3 Polynucleotide Compositions

Any polynucleotide that encodes a WT1 peptide as described herein, or that is complementary to such a polynucleotide, is a WT1 polynucleotide encompassed by the present invention. Such polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials.

WT1 polynucleotides may encode a native WT1 protein, or may encode a variant of WT1 as described herein. Polynucleotide variants may contain one or more substitutions, additions, deletions and/or insertions such that the immunogenicity of the encoded peptide is not diminished, relative to a native WT1 protein. The effect on the immunogenicity of the encoded peptide may generally be assessed as described herein. Preferred peptide variants contain amino acid substitutions, deletions, insertions and/or additions at no more than about 20%, more preferably at no more than about 15%, and more preferably still, at no more than about 10% or 5% or less of the amino acid positions relative to the corresponding native unmodified WT1 sequence.

Likewise, polynucleotides encoding such peptide variants should preferably contain nucleotide substitutions, deletions, insertions and/or additions at no more than about 20%, more preferably at no more than about 15%, and more preferably still, at no more than about 10% or 5% or less of the nucleotide positions relative to the corresponding polynucleotide sequence that encodes the native unmodified WT1 peptide sequence. Certain polynucleotide variants, of course, may be substantially homologous to, or substantially identical to the corresponding region of the nucleotide sequence encoding an unmodified peptide. Such polynucleotide variants are capable of hybridizing to a naturally occurring DNA sequence encoding a WT1 peptide (or a complementary sequence) under moderately stringent, to highly stringent, to very highly stringent conditions.

Suitable moderately stringent conditions include prewashing in a solution containing about 5X SSC, 0.5% SDS, 1.0 mM EDTA (pH 8.0); hybridizing at a temperature of from about 50° C. to about 60° C. in 5× SSC overnight; followed by washing twice at about 60 to 65° C. for 20 min. with each of 2×, 0.5× and 0.2× SSC containing 0.1% SDS). Suitable highly stringent conditions include prewashing in a solution containing about 5× SSC, 0.5% SDS, 1.0 mM EDTA (pH 8.0); hybridizing at a temperature of from about 60° C. to about 70° C. in 5× SSC overnight; followed by washing twice at about 65 to 70° C. for 20 min. with each of 2×, 0.5× and 0.2× SSC containing 0.1% SDS). Representative examples of very highly stringent hybridization conditions may include, for example, prewashing in a solution containing about 5× SSC, 0.5% SDS, 1.0 mM EDTA (pH 8.0); hybridizing at a temperature of from about 70° C. to about 75° C. in 5× SSC overnight; followed by washing twice at about 70° C. to about 75° C. for 20 min. with each of 2×, 0.5× and 0.2× SSC containing 0.1% SDS). Such hybridizing DNA sequences are also within the scope of this invention.

It will be appreciated by those of ordinary skill in the art that, as a result of the degeneracy of the genetic code, there are many nucleotide sequences that encode a WT1, peptide. Some of these polynucleotides bear minimal homology to the nucleotide sequence of any native gene. Nonetheless, polynucleotides that vary due to differences in codon usage are specifically contemplated by the present invention.

Once an immunogenic portion of WT1 is identified, as described above, a WT1 polynucleotide may be prepared using any of a variety of techniques. For example, a WT1 polynucleotide may be amplified from cDNA prepared from cells that express WT1. Such polynucleotides may be amplified via polymerase chain reaction (PCR™). For this approach, sequence-specific primers may be designed based on the sequence of the immunogenic portion and may be purchased or synthesized.

For example, suitable primers for PCR™ amplification of a human WT1 gene include: first step—P118:1434-1414: 5′-GAGAGTCAGACTTGAAAGCAGT-3′ (SEQ ID NO:5) and P135: 5′-CTGAGCCTCAGCAAATGGGC-3′ (SEQ ID NO:6); second step—P136: 5′-GAGCATGCATGGGCTCCGACGTGCGGG-3′ (SEQ ID NO:7) and P137: 5′-GGGGTACCCACTGAACGGTCCCCGA-3′(SEQ ID NO:8). Primers for PCR™ amplification of a mouse WT1 gene include: first step—P138: 5′-TCCGAGCCGCACCTCATG-3′ (SEQ ID NO:9) and P139:5′-GCCTGGGATGCTGGACTG-3′ (SEQ ID NO:10), second step—P140: 5′-GAGCATGCGATGGGTTCCGACGTGCGG-3′ (SEQ ID NO:11) and P141: 5′-GGGGTACCTCAAAGCGCCACGTGGAGTTT-3′ (SEQ ID NO:12).

An amplified portion may then be used to isolate a full-length gene from a human genomic DNA library or from a suitable cDNA library, using well-known techniques. Alternatively, a full-length gene can be constructed from multiple PCR™ fragments. WT1 polynucleotides may also be prepared by synthesizing oligonucleotide components, and ligating components together to generate the complete polynucleotide.

WT1 polynucleotides may also be synthesized by any method known in the art, including chemical synthesis (e.g., solid phase phosphoramidite chemical synthesis). Modifications in a polynucleotide sequence may also be introduced using standard mutagenesis techniques, such as oligonucleotide-directed site-specific mutagenesis (Adelman et al., 1983). Alternatively, RNA molecules may be generated by in vitro or in vivo transcription of DNA sequences encoding a WT1 peptide, provided that the DNA is incorporated into a vector with a suitable RNA polymerase promoter (such as T7 or SP6). Certain portions may be used to prepare an encoded peptide, as described herein. In addition, or alternatively, a portion may be administered to a patient such that the encoded peptide is generated in vivo (e.g., by transfecting antigen-presenting cells such as dendritic cells with a cDNA construct encoding a WT1 peptide, and administering the transfected cells to the patient).

Polynucleotides that encode a WT1 peptide may generally be used for production of the peptide, in vitro or in vivo. WT1 polynucleotides that are complementary to a coding sequence (i.e., antisense polynucleotides) may also be used as a probe or to inhibit WT1, expression. cDNA constructs that can be transcribed into antisense RNA may also be introduced into cells of tissues to facilitate the production of antisense RNA.

Any polynucleotide may be further modified to increase stability in vivo. Possible modifications include, but are not limited to, the addition of flanking sequences at the 5′ and/or 3′-ends; the use of phosphorothioate or 2′-o-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Nucleotide sequences as described herein may be joined to a variety of other nucleotide sequences using established recombinant DNA techniques. For example, a polynucleotide may be cloned into any of a variety of cloning vectors, including plasmids, phagemids, lambda phage derivatives and cosmids. Vectors of particular interest include expression vectors, replication vectors, probe generation vectors, and sequencing vectors. In general, a vector will contain an origin of replication functional in at least one organism, convenient restriction endonuclease sites and one or more selectable markers. Other elements will depend upon the desired use, and will be apparent to those of ordinary skill in the art.

Within certain embodiments, polynucleotides may be formulated so as to permit entry into a cell of a mammal, and expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno-associated virus, retrovirus, or vaccinia or other poxvirus (e.g., avian poxvirus). Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art. cDNA constructs within such a vector may be used, for example, to transfect human or animal cell lines for use in establishing WT1 positive tumor models which may be used to perform tumor protection and adoptive immunotherapy experiments to demonstrate tumor or leukemia-growth inhibition or lysis of such cells.

Other therapeutic formulations for polynucleotides include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

4.4 Methods of Nucleic Acid Delivery and DNA Transfection

In certain embodiments, it is contemplated that one or more RNA or DNA and/or substituted polynucleotide compositions disclosed herein will be used to transfect an appropriate host cell. Technology for introduction of RNAs and DNAs, and vectors comprising them into suitable host cells is well known to those of skill in the art. In particular, such polynucleotides may be used to genetically transform one or more host cells, when therapeutic administration of one or more active peptides, compounds or vaccines is achieved through the expression of one or more polynucleotide constructs that encode one or more therapeutic compounds of interest.

A variety of means for introducing polynucleotides and/or polypeptides into suitable target cells is known to those of skill in the art. For example, when polynucleotides are contemplated for delivery to cells, several non-viral methods for the transfer of expression constructs into cultured mammalian cells are available to the skilled artisan for his use. These include, for example, calcium phosphate precipitation (Graham and Van Der Eb, 1973; Chen and Okayama, 1987; Rippe et al., 1990); DEAE-dextran precipitation (Gopal, 1985); electroporation (Wong and Neumann, 1982; Fronum et al., 1985; Tur-Kaspa et al., 1986; Potter et al., 1984; Suzuki et al., 1998; Vanbever et al., 1998), direct microinjection (Capecchi, 1980; Harland and Weintraub, 1985), DNA-loaded liposomes (Nicolau and Sene, 1982; Fraley et al., 1979; Takakura, 1998) and lipofectamine-DNA complexes, cell sonication (Fechheimer et al., 1987), gene bombardment using high velocity microprojectiles (Yang et al., 1990; Klein et al., 1992), and receptor-mediated transfection (Curiel et al., 1991; Wagner et al., 1992; Wu and Wu, 1987; Wu and Wu, 1988). Some of these techniques may be successfully adapted for in vivo or ex vivo use.

A bacterial cell, a yeast cell, or an animal cell transformed with one or more of the disclosed expression vectors represent an important aspect of the present invention. Such transformed host cells are often desirable for use in the expression of the various DNA gene constructs disclosed herein. In some aspects of the invention, it is often desirable to modulate, regulate, or otherwise control the expression of the gene segments disclosed herein. Such methods are routine to those of skill in the molecular genetic arts. Typically, when increased or over-expression of a particular gene is desired, various manipulations may be employed for enhancing the expression of the messenger RNA, particularly by using an active promoter, and in particular, a tissue-specific promoter such as those disclosed herein, as well as by employing sequences, which enhance the stability of the messenger RNA in the particular transformed host cell.

Typically, the initiation and translational termination region will involve stop codon(s), a terminator region, and optionally, a polyadenylation signal. In the direction of transcription, namely in the 5′ to 3′ direction of the coding or sense sequence, the construct will involve the transcriptional regulatory region, if any, and the promoter, where the regulatory region may be either 5′ or 3′ of the promoter, the ribosomal binding site, the initiation codon, the structural gene having an open reading frame in phase with the initiation codon, the stop codon(s), the polyadenylation signal sequence, if any, and the terminator region. This sequence as a double strand may be used by itself for transformation of a microorganism or eukaryotic host, but will usually be included with a DNA sequence involving a marker, where the second DNA sequence may be joined to the expression construct during introduction of the DNA into the host.

Where no functional replication system is present, the construct will also preferably include a sequence of at least about 30 or about 40 or about 50 basepairs (bp) or so, preferably at least about 60, about 70, about 80, or about 90 to about 100 or so bp, and usually not more than about 500 to about 1000 or so bp of a sequence homologous with a sequence in the host. In this way, the probability of legitimate recombination is enhanced, so that the gene will be integrated into the host and stably maintained by the host. Desirably, the regulatory regions of the expression construct will be in close proximity to (and also operably positioned relative to) the selected therapeutic gene providing for complementation as well as the gene providing for the competitive advantage. Therefore, in the event that the therapeutic gene is lost, the resulting organism will be likely to also lose the gene providing for the competitive advantage, so that it will be unable to compete in the environment with the gene retaining the intact construct.

The selected therapeutic gene can be introduced between the transcriptional and translational initiation region and the transcriptional and translational termination region, so as to be under the regulatory control of the initiation region. This construct may be included in a plasmid, which will include at least one replication system, but may include more than one, where one replication system is employed for cloning during the development of the plasmid and the second replication system is necessary for functioning in the ultimate host, in this case, a mammalian host cell. In addition, one or more markers may be present, which have been described previously. Where integration is desired, the plasmid will desirably include a sequence homologous with the host genome.

Genes or other nucleic acid segments, as disclosed herein, can be inserted into host cells using a variety of techniques that are well known in the art. Five general methods for delivering a nucleic segment into cells have been described: (1) chemical methods (Graham and VanDerEb, 1973); (2) physical methods such as microinjection (Capecchi, 1980), electroporation (U.S. Pat. No. 5,472,869; Wong and Neumann, 1982; Fromm et al., 1985), microprojectile bombardment (U.S. Pat. No. 5,874,265, specifically incorporated herein by reference in its entirety), “gene gun” (Yang et al., 1990); (3) viral vectors (Eglitis and Anderson, 1988); (4) receptor-mediated mechanisms (Curiel et al., 1991; Wagner et al, 1992); and (5) bacterial-mediated transformation.

4.5 WT1-specific Antibodies and Antigen-binding Fragments Thereof

The present invention further provides antibodies and antigen-binding fragments thereof, that specifically bind to (or are immunospecific for) at least a first peptide or peptide variant as disclosed herein. As used herein, an antibody or an antigen-binding fragment is said to “specifically bind” to a peptide if it reacts at a detectable level (within, for example, an ELISA) with the peptide, and does not react detectably with unrelated peptides or proteins under similar conditions. As used herein, “binding” refers to a noncovalent association between two separate molecules such that a “complex” is formed. The ability to bind may be evaluated by, for example, determining a binding constant for the formation of the complex. The binding constant is the value obtained when the concentration of the complex is divided by the product of the component concentrations. In the context of the present invention, in general, two compounds are said to “bind” when the binding constant for complex formation exceeds about 10³ L/mol. The binding constant maybe determined using methods well known in the art.

Any agent that satisfies the above requirements may be a binding agent. In illustrative embodiments, a binding agent is an antibody or an antigen-binding fragment thereof. Such antibodies may be prepared by any of a variety of techniques known to those of ordinary skill in the art (Harlow and Lane, 1988). In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the peptide is initially injected into any of a wide variety of mammals (e.g., mice, rats, rabbits, sheep or goats). In this step, the peptides of this invention may serve as the immunogen without modification. Alternatively, particularly for relatively short peptides, a superior immune response may be elicited if the peptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the peptide may then be purified from such antisera by, for example, affinity chromatography using the peptide coupled to a suitable solid support.

Monoclonal antibodies specific for the antigenic peptide of interest may be prepared, for example, using the technique of Kohler and Milstein (1976) and improvements thereto. Briefly, these methods involve the preparation of immortal cell lines capable of producing antibodies having the desired specificity (i.e., reactivity with the peptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the peptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The peptides of this invention may be used in the purification process in, for example, an affinity chromatography step.

Within certain embodiments, the use of antigen-binding fragments of antibodies may be preferred. Such fragments include Fab fragments, which may be prepared using standard techniques. Briefly, immunoglobulins may be purified from rabbit serum by affinity chromatography on Protein A bead columns (Harlow and Lane, 1988) and digested by papain to yield Fab and Fc fragments. The Fab and Fc fragments may be separated by affinity chromatography on Protein A bead columns.

Monoclonal antibodies and fragments thereof may be coupled to one or more therapeutic agents. Suitable agents in this regard include radioactive tracers and chemotherapeutic agents, which may be used, for example, to purge autologous bone marrow in vitro). Representative therapeutic agents include radionuclides, differentiation inducers, drugs, toxins, and derivatives thereof. Preferred radionuclides include ⁹⁰Y, ¹²³I, ¹²⁵I, ¹³¹I, ¹⁸⁶Re, ¹⁸⁸Re, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein. For diagnostic purposes, coupling of radioactive agents may be used to facilitate tracing of metastases or to determine the location of WT1-positive tumors.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be affected, for example, through amino groups, carboxyl groups, and sulfhydryl groups or oxidized carbohydrate residues. There are numerous references describing such methodology, e.g., U.S. Pat. No. 4,671,958.

Where a therapeutic agent is more potent when free from the antibody portion of the immunoconjugates of the present invention, it may be desirable to use a linker group that is cleavable during or upon internalization into a cell. A number of different cleavable linker groups have been described. The mechanisms for the intracellular release of an agent from these linker groups include cleavage by reduction of a disulfide bond (U.S. Pat. No. 4,489,710), by irradiation of a photolabile bond (U.S. Pat. No. 4,625,014), by hydrolysis of derivatized amino acid side chains (U.S. Pat. No. 4,638,045), by serum complement-mediated hydrolysis (U.S. Pat. No. 4,671,958), and acid-catalyzed hydrolysis (U.S. Pat. No. 4,569,789).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers that provide multiple sites for attachment can be used. Alternatively, a carrier can be used. A carrier may bear the agents in a variety of ways, including covalent bonding either directly or via a linker group. Suitable carriers include proteins such as albumins (U.S. Pat. No. 4,507,234), peptides and polysaccharides such as aminodextran (U.S. Pat. No. 4,699,784). A carrier may also bear an agent by noncovalent bonding or by encapsulation, such as within a liposome vesicle (U.S. Pat. No. 4,429,008 and U.S. Pat. No. 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562 discloses representative chelating compounds and their synthesis.

A variety of routes of administration for the antibodies and immunoconjugates may be used. Typically, administration will be intravenous, intramuscular, subcutaneous or in the bed of a resected tumor. It will be evident that the precise dose of the antibody/ immuno-conjugate will vary depending upon the antibody used, the antigen density on the tumor, and the rate of clearance of the antibody.

Also provided herein are anti-idiotypic antibodies that mimic an immunogenic portion of WT1. Such antibodies may be raised against an antibody, or an antigen-binding fragment thereof, that specifically binds to an immunogenic portion of WT1, using well-known techniques. Anti-idiotypic antibodies that mimic an immunogenic portion of WT1, are those antibodies that bind to an antibody, or antigen-binding fragment thereof, that specifically binds to an immunogenic portion of WT1, as described herein.

Irrespective of the source of the original WT1 peptide-specific antibody, the intact antibody, antibody multimers, or any one of a variety of functional, antigen-binding regions of the antibody may be used in the present invention. Exemplary functional regions include scFv, Fv, Fab′, Fab and F(ab′)₂ fragments of the WT1 peptide-specific antibodies. Techniques for preparing such constructs are well known to those in the art and are further exemplified herein.

The choice of antibody construct may be influenced by various factors. For example, prolonged half-life can result from the active readsorption of intact antibodies within the kidney, a property of the Fc piece of immunoglobulin. IgG based antibodies, therefore, are expected to exhibit slower blood clearance than their Fab′ counterparts. However, Fab′ fragment-based compositions will generally exhibit better tissue penetrating capability.

Antibody fragments can be obtained by proteolysis of the whole immunoglobulin by the non-specific thiol protease, papain. Papain digestion yields two identical antigen-binding fragments, termed “Fab fragments,” each with a single antigen-binding site, and a residual “Fc fragment.”

Papain should first be activated by reducing the sulphydryl group in the active site with cysteine, 2-mercaptoethanol or dithiothreitol. Heavy metals in the stock enzyme should be removed by chelation with EDTA (2 mM) to ensure maximum enzyme activity. Enzyme and substrate are normally mixed together in the ratio of 1:100 by weight. After incubation, the reaction can be stopped by irreversible alkylation of the thiol group with iodoacetamide or simply by dialysis. The completeness of the digestion should be monitored by SDS-PAGE and the various fractions separated by Protein A-Sepharose or ion exchange chromatography.

The usual procedure for preparation of F(ab′)₂ fragments from IgG of rabbit and human origin is limited proteolysis by the enzyme pepsin. The conditions, 100× antibody excess wt./wt. in acetate buffer at pH 4.5, 37° C., suggest that antibody is cleaved at the C-terminal side of the inter-heavy-chain disulfide bond. Rates of digestion of mouse IgG may vary with subclass and it may be difficult to obtain high yields of active F(ab′)₂ fragments without some undigested or completely degraded IgG. In particular, IgG₂L is highly susceptible to complete degradation. The other subclasses require different incubation conditions to produce optimal results, all of which is known in the art.

Pepsin treatment of intact antibodies yields an F(ab′)₂ fragment that has two antigen-combining sites and is still capable of cross-linking antigen. Digestion of rat IgG by pepsin requires conditions including dialysis in 0.1 M acetate buffer, pH 4.5, and then incubation for four hrs with 1% wt./wt. pepsin; IgG₁ and IgG₂ₐ digestion is improved if first dialyzed against 0.1 M formate buffer, pH 2.8, at 4° C., for 16 hrs followed by acetate buffer. IgG_{2b} gives more consistent results with incubation in staphylococcal V8 protease (3% wt./wt.) in 0.1 M sodium phosphate buffer, pH 7.8, for four hrs at 37° C.

A Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. F(ab′)₂ antibody fragments were originally produced as pairs of Fab′ fragments that have hinge cysteines between them. Other chemical couplings of antibody fragments are also known.

The term “variable,” as used herein in reference to antibodies, means that certain portions of the variable domains differ extensively in sequence among antibodies, and are used in the binding and specificity of each particular antibody to its particular antigen. However, the variability is not evenly distributed throughout the variable domains of antibodies. It is concentrated in three segments termed “hypervariable regions,” both in the light chain and the heavy chain variable domains.

The more highly conserved portions of variable domains are called the framework region (FR). The variable domains of native heavy and light chains each comprise four FRs (FR1, FR2, FR3 and FR4, respectively), largely adopting a β-sheet configuration, connected by three hypervariable regions, which form loops connecting, and in some cases, forming part of, the β-sheet structure.

The hypervariable regions in each chain are held together in close proximity by the FRs and, with the hypervariable regions from the other chain, contribute to the formation of the antigen-binding site of antibodies (Kabat etal., 1991, specifically incorporated herein by reference). The constant domains are not involved directly in binding an antibody to an antigen, but exhibit various effector functions, such as participation of the antibody in antibody-dependent cellular toxicity.

The term “hypervariable region,” as used herein, refers to the amino acid residues of an antibody that are responsible for antigen binding. The hypervariable region comprises amino acid residues from a “complementarity determining region” or “CDR” (i.e. residues 24-34 (LI), 50-56 (L2) and 89-97 (L3) in the light chain variable domain and 31-35 (HI), 50-56 (H2) and 95-102 (H3) in the heavy chain variable domain (Kabat et al., 1991, specifically incorporated herein by reference) and/or those residues from a “hypervariable loop” (i.e., residues 26-32 (LI), 50-52(L2) and 91-96 (L3) in the light chain variable domain and 26-32 (Hi), 53-55 (H2) and 96-101 (H3) in the heavy chain variable domain). “Framework” or “FR” residues are those variable domain residues other than the hypervariable region residues as herein defined.

An “Fv” fragment is the minimum antibody fragment that contains a complete antigen-recognition and binding site. This region consists of a dimer of one heavy chain and one light chain variable domain in tight, con-covalent association. It is in this configuration that three hypervariable regions of each variable domain interact to define an antigen-binding site on the surface of the V_{H}-V_{L} dimer. Collectively, six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

“Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains that enables the sFv to form the desired structure for antigen binding.

“Diabodies” are small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described in European Pat. Appl. No. EP 404,097 and Intl. Pat. Appl. Publ. No. WO 93/11161, each specifically incorporated herein by reference. “Linear antibodies”, which can be bispecific or monospecific, comprise a pair of tandem Fd segments (V_{H}-C_{H}1-V_{H}-C_{H}1) that form a pair of antigen binding regions, as described in Zapata et al. (1995), specifically incorporated herein by reference.

Other types of variants are antibodies with improved biological properties relative to the parent antibody from which they are generated. Such variants, or second-generation compounds, are typically substitutional variants involving one or more substituted hypervariable region residues of a parent antibody. A convenient way for generating such substitutional variants is affinity maturation using phage display.

In affinity maturation using phage display, several hypervariable region sites (e.g., 6 to 7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g, binding affinity) as herein disclosed. In order to identify candidate hypervariable region sites for modification, alanine-scanning mutagenesis can be performed on hypervariable region residues identified as contributing significantly to antigen binding.

Alternatively, or in addition, the crystal structure of the antigen-antibody complex be delineated and analyzed to identify contact points between the antibody and target. Such contact residues and neighboring residues are candidates for substitution. Once such variants are generated, the panel of variants is subjected to screening, and antibodies with analogues but different or even superior properties in one or more relevant assays are selected for firther development.

In using a Fab′ or antigen binding fragment of an antibody, with the attendant benefits on tissue penetration, one may derive additional advantages from modifying the fragment to increase its half-life. A variety of techniques may be employed, such as manipulation or modification of the antibody molecule itself, and also conjugation to inert carriers. Any conjugation for the sole purpose of increasing half-life, rather than to deliver an agent to a target, should be approached carefuilly in that Fab′ and other fragments are chosen to penetrate tissues. Nonetheless, conjugation to non-protein polymers, such PEG and the like, is contemplated.

Modifications other than conjugation are therefore based upon modifying the structure of the antibody fragment to render it more stable, and/or to reduce the rate of catabolism in the body. One mechanism for such modifications is the use of D-amino acids in place of L-amino acids. Those of ordinary skill in the art will understand that the introduction of such modifications needs to be followed by rigorous testing of the resultant molecule to ensure that it still retains the desired biological properties. Further stabilizing modifications include the use of the addition of stabilizing moieties to either the N-terninal or the C-terminal, or both, which is generally used to prolong the half-life of biological molecules. By way of example only, one may wish to modify the termini by acylation or amination.

Moderate conjugation-type modifications for use with the present invention include incorporating a salvage receptor binding epitope into the antibody fragment. Techniques for achieving this include mutation of the appropriate region of the antibody fragment or incorporating the epitope as a peptide tag that is attached to the antibody fragment. Intl. Pat. Appl. Publ. No. WO 96/32478 is specifically incorporated herein by reference for the purposes of further exemplifying such technology. Salvage receptor binding epitopes are typically regions of three or more amino acids from one or two lops of the Fc domain that are transferred to the analogous position on the antibody fragment. The salvage receptor-binding epitopes disclosed in Intl. Pat. Appl. Publ. No. WO 98/45331, are incorporated herein by reference for use with the present invention.

4.6 T Cell Compositions Specific for WT1 Peptides

Immunotherapeutic compositions may also, or alternatively, comprise T cells specific for WT1. Such cells may generally be prepared in vitro or ex vivo, using standard procedures. For example, T cells may be present within (or isolated from) bone marrow, peripheral blood or a fraction of bone marrow or peripheral blood of a mammal, such as a patient, using a commercially available cell separation system, such as the Isolex™ System, available from Nexell Therapeutics, Inc. (Irvine, Calif.; see also U.S. Pat. No. 5,240,856; U.S. Pat. No. 5,215,926; Intl. Pat. Appl. Publ. No. WO 89/06280; Intl. Pat. Appl. Publ. No. WO 91/16116 and Intl. Pat. Appl. Publ. No. WO 92/07243). Alternatively, T cells may be derived from related or unrelated humans, non-human mammals, cell lines or cultures.

T cells may be stimulated with WT1 peptide, polynucleotide encoding a WT1 peptide and/or an antigen-presenting cell (APC) that expresses a WT1 peptide. Such stimulation is performed under conditions and for a time sufficient to permit the generation of T cells that are specific for the WT1 peptide. Preferably, a WT1 peptide or polynucleotide is present within a delivery vehicle, such as a microsphere, to facilitate the generation of antigen-specific T cells. Briefly, T cells, which may be isolated from a patient or a related or unrelated donor by routine techniques (such as by Ficoll/Hypaque® density gradient centrifugation of peripheral blood lymphocytes), are incubated with WT1 peptide. For example, T cells may be incubated in vitro for 2-9 days (typically 4 days) at 37° C. with WT1, peptide (e.g., 5 to 25 tg/ml) or cells synthesizing a comparable amount of WT1 peptide. It may be desirable to incubate a separate aliquot of a T cell sample in the absence of WT1, peptide to serve as a control.

T cells are considered to be specific for a WT1 peptide if the T cells kill target cells coated with a WT1 peptide or expressing a gene encoding such a peptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al. (1994). Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Other ways to detect T cell proliferation include measuring increases in interleukin-2 (IL-2) production, Ca²⁺ flux, or dye uptake, such as 3-(4,5-dimethylthiazol-2-yl)-2,5-diphenyl-tetrazolium. Alternatively, synthesis of lymphokines (such as interferon-gamma) can be measured or the relative number of T cells that can respond to a WT1 peptide may be quantified. Contact with a WT1 peptide (200 ng/ml-100 μg/ml, preferably 100 ng/ml-25 μg/ml) for 3-7 days should result in at least a two-fold increase in proliferation of the T cells and/or contact as described above for 2-3 hrs should result in activation of the T cells, as measured using standard cytokine assays in which a two-fold increase in the level of cytokine release (e.g., TNF or IFN-γ) is indicative of T cell activation (Coligan et al., 1998). WT1, specific T cells may be expanded using standard techniques. Within preferred embodiments, the T cells are derived from a patient or a related or unrelated donor and are administered to the patient following stimulation and expansion.

T cells that have been activated in response to a WT1 peptide, polynucleotide or WT1-expressing APC may be CD4⁺ and/or CD8⁺. Specific activation of CD4⁺ or CD8⁺ T cells may be detected in a variety of ways. Methods for detecting specific T cell activation include detecting the proliferation of T cells, the production of cytokines (e.g., lymphokines), or the generation of cytolytic activity (i.e., generation of cytotoxic T cells specific for WT1). For CD4⁺ T cells, a preferred method for detecting specific T cell activation is the detection of the proliferation of T cells. For CD8⁺ T cells, a preferred method for detecting specific T cell activation is the detection of the generation of cytolytic activity.

For therapeutic purposes, CD4+or CD8⁺ T cells that proliferate in response to the WT1 peptide, polynucleotide or APC can be expanded in number either in vitro or in vivo. Proliferation of such T cells in vitro may be accomplished in a variety of ways. For example, the T cells can be re-exposed to WT1 peptide, with or without the addition of T cell growth factors, such as interleukin-2, and/or stimulator cells that synthesize a WT1 peptide. The addition of stimulator cells is preferred where generating CD8⁺ T cell responses. T cells can be grown to large numbers in vitro with retention of specificity in response to intermittent restimulation with WT1 peptide. Briefly, for the primary in vitro stimulation (IVS), large numbers of lymphocytes (e.g., greater than 4×10⁷) may be placed in flasks with media containing human serum. WT1 peptide (e.g., peptide at 10 μg/ml) may be added directly, along with tetanus toxoid (e.g., 5 μg/ml). The flasks may then be incubated (e.g., 37° C. for 7 days). For a second IVS, T cells are then harvested and placed in new flasks with 2-3×10⁷ irradiated peripheral blood mononuclear cells. WT1 peptide (e.g., 10 μg/ml) is added directly. The flasks are incubated at 37° C. for 7 days. On day 2 and day 4 after the second IVS, 2-5 units of interleukin-2 (IL-2) may be added. For a third IVS, the T cells may be placed in wells and stimulated with the individual's own EBV transformed B cells coated with the peptide. IL-2 may be added on days 2 and 4 of each cycle. As soon as the cells are shown to be specific cytotoxic T cells, they may be expanded using a 10-day stimulation cycle with higher IL-2 (20 units) on days 2, 4 and 6.

Alternatively, one or more T cells that proliferate in the presence of WT1 peptide can be expanded in number by cloning. Methods for cloning cells are well known in the art, and include limiting dilution. Responder T cells may be purified from the peripheral blood of sensitized patients by density gradient centrifugation and sheep red cell rosetting and established in culture by stimulating with the nominal antigen in the presence of irradiated autologous filler cells. In order to generate CD4⁺ T cell lines, WT1 peptide is used as the antigenic stimulus and autologous peripheral blood lymphocytes (PBL) or lymphoblastoid cell lines (LCL) immortalized by infection with Epstein Barr virus are used as antigen-presenting cells. In order to generate CD8⁺ T cell lines, autologous antigen-presenting cells transfected with an expression vector that produces WT1 peptide may be used as stimulator cells. Established T cell lines may be cloned 2-4 days following antigen stimulation by plating stimulated T cells at a frequency of 0.5 cells per well in 96-well flat-bottom plates with 1×10⁶ irradiated PBL or LCL cells and recombinant interleukin-2 (rIL2) (50 U/ml). Wells with established clonal growth may be identified at approximately 2-3 weeks after initial plating and restimulated with appropriate antigen in the presence of autologous antigen-presenting cells, then subsequently expanded by the addition of low doses of rIL2 (10 U/ml) 2-3 days following antigen stimulation. T cell clones may be maintained in 24-well plates by periodic restimulation with antigen and rIL2 approximately every two weeks. Cloned and/or expanded cells may be administered back to the patient as described, for example, by Chang et al., (1996).

Within certain embodiments, allogeneic T-cells may be primed (i.e., sensitized to WT1,) in vivo and/or in vitro. Such priming may be achieved by contacting T cells with a WT1 peptide, a polynucleotide encoding such a peptide or a cell producing such a peptide under conditions and for a time sufficient to permit the priming of T cells. In general, T cells are considered to be primed if, for example, contact with a WT1 peptide results in proliferation and/or activation of the T cells, as measured by standard proliferation, chromium release and/or cytokine release assays as described herein. A stimulation index of more than two fold increase in proliferation or lysis, and more than three fold increase in the level of cytokine, compared to negative controls indicates T-cell specificity. Cells primed in vitro may be employed, for example, within bone marrow transplantation or as donor lymphocyte infusion.

T cells specific for WT1 can kill cells that express WT1 protein. Introduction of genes encoding T-cell receptor (TCR) chains for WT1 are used as a means to quantitatively and qualitatively improve responses to WT1 bearing leukemia and cancer cells. Vaccines to increase the number of T cells that can react to WT1 positive cells are one method of targeting WT1 bearing cells. T cell therapy with T cells specific for WT1 is another method. An alternative method is to introduce the TCR chains specific for WT1 into T cells or other cells with lytic potential. In a suitable embodiment, the TCR alpha and beta chains are cloned out from a WT1 specific T cell line and used for adoptive T cell therapy, such as described in WO96/30516, incorporated herein by reference.

4.7 Pharmaceutical Compositions and Vaccine Formulations

Within certain aspects, peptides, polynucleotides, antibodies and/or T cells may be incorporated into pharmaceutical compositions or immunogenic compositions (i.e., vaccines). Alternatively, a pharmaceutical composition may comprise an antigen-presenting cell (e.g., a dendritic cell) transfected with a WT1 polynucleotide such that the antigen-presenting cell expresses a WT1 peptide. Pharmaceutical compositions comprise one or more such compounds or cells and a physiologically acceptable carrier or excipient. Vaccines may comprise one or more such compounds or cells and an immunostimulant, such as an adjuvant or a liposome (into which the compound is incorporated). An immunostimulant may be any substance that enhances or potentiates an immune response (antibody- and/or cell-mediated) to an exogenous antigen. Examples of immunostimulants include adjuvants, biodegradable microspheres (e.g., polylactic galactide) and liposomes (into which the compound is incorporated) (U.S. Pat. No. 4,235,877). Vaccine preparation is generally described in, for example, Powell and Newman (1995). Pharmaceutical compositions and vaccines within the scope of the present invention may also contain other compounds, which may be biologically active or inactive. For example, one or more immunogenic portions of other tumor antigens may be present, either incorporated into a fusion peptide or as a separate compound, within the composition or vaccine.

Within certain embodiments, pharmaceutical compositions and vaccines are designed to elicit T cell responses specific for a WT1 peptide in a patient, such as a human. In general, T cell responses may be favored through the use of relatively short peptides (e.g., comprising less than 23 consecutive amino acid residues of a native WT1 peptide, preferably 4-16 consecutive residues, more preferably 8-16 consecutive residues and still more preferably 8-10 consecutive residues). Alternatively, or in addition, a vaccine may comprise an immunostimulant that preferentially enhances a T cell response. In other words, the immunostimulant may enhance the level of a T cell response to a WT1 peptide by an amount that is proportionally greater than the amount by which an antibody response is enhanced. For example, when compared to a standard oil based adjuvant, such as CFA, an immunostimulant that preferentially enhances a T cell response may enhance a proliferative T cell response by at least two fold, a lytic response by at least 10%, and/or T cell activation by at least two fold compared to WT1-negative control cell lines, while not detectably enhancing an antibody response. The amount by which a T cell or antibody response to a WT1 peptide is enhanced may generally be determined using any representative technique known in the art, such as the techniques provided herein.

A pharmaceutical composition or vaccine may contain DNA encoding one or more of the peptides as described above, such that the peptide is generated in situ. As noted above, the DNA may be present within any of a variety of delivery systems known to those of ordinary skill in the art, including nucleic acid expression systems, bacterial and viral expression systems and mammalian expression systems. Numerous gene delivery techniques are well known in the art (Rolland, 1998, and references cited therein). Appropriate nucleic acid expression systems contain the necessary DNA, cDNA or RNA sequences for expression in the patient (such as a suitable promoter and terminating signal). Bacterial delivery systems involve the administration of a bacterium (such as Bacillus-Calmette-Guerrin) that expresses an immunogenic portion of the peptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus (Fisher-Hoch et al., 1989; Flexner et al., 1989; Flexner et al., 1990; U.S. Pat. No. 4,603,112, U.S. Pat. No. 4,769,330, and U.S. Pat. No. 5,017,487; Intl. Pat. Appl. Publ. No. WO 89/01973; U.S. Pat. No. 4,777,127; Great Britain Pat. No. GB 2,200,651; European Pat. No. EP 0,345,242; Intl. Pat. Appl. Publ. No. WO 91/02805; Berkner, 1988; Rosenfeld et al., 1991; Kolls et al., 1994; Kass-Eisler et al., 1993; Guzman et al., 1993a; and Guzman et al., 1993). Techniques for incorporating DNA into such expression systems are well known to those of ordinary skill in the art. The DNA may also be “naked,” as described, for example, in Ulmer et aL (1993) and reviewed by Cohen (1993). The uptake of naked DNA may be increased by coating the DNA onto biodegradable beads, which are efficiently transported into the cells. It will be apparent that a vaccine may comprise both a polynucleotide and a peptide component. Such vaccines may provide for an enhanced immune response.

As noted above, a pharmaceutical composition or vaccine may comprise an antigen-presenting cell that expresses a WT1 peptide. For therapeutic purposes, as described herein, the antigen-presenting cell is preferably an autologous dendritic cell. Such cells may be prepared and transfected using standard techniques (Reeves et al., 1996; Tuting et al., 1998; and Nair et al., 1998). Expression of a WT1 peptide on the surface of an antigen-presenting cell may be confirmed by in vitro stimulation and standard proliferation as well as chromium release assays, as described herein.

It will be apparent to those of ordinary skill in the art having the benefit of the present teachings that a vaccine may contain pharmaceutically acceptable salts of the polynucleotides and peptides provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts). The phrases “pharmaceutically or pharmacologically acceptable” refer to molecular entities and compositions that do not produce an adverse, allergic or other significant untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations should meet sterility, pyrogenicity, and general safety and purity standards as required by the Food and Drug Administration Office of Biologics standards. Supplementary active ingredients can also be incorporated into the compositions.

While any suitable carrier known to those of ordinary skill in the art may be employed in the pharmaceutical compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252. For certain topical applications, formulation as a cream or lotion, using well-known components, is preferred.

Such compositions may also comprise buffers (e.g., neutral buffered saline or phosphate buffered saline), carbohydrates (e.g., glucose, mannose, sucrose or dextrans), mannitol, proteins, peptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate, or formulated with one or more liposomes, microspheres, nanoparticles, or micronized delivery systems using well-known technology.

Any of a variety of immunostimulants, such as adjuvants, may be employed in the preparation of vaccine compositions of this invention. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, alum-based adjuvants (e.g, Alhydrogel, Rehydragel, aluminum phosphate, Algammulin, aluminum hydroxide); oil based adjuvants (Freund's Incomplete Adjuvant and Complete Adjuvant (Difco Laboratories, Detroit, Mich.), Specol, RIBI, TiterMax, Montanide ISA50 or Seppic MONTANIDE ISA 720); nonionic block copolymer-based adjuvants, cytokines (e.g., GM-CSF or Flat3-ligand); Merck Adjuvant 65 (Merck and Company, Inc., Rahway, N.J.); AS-2 (SmithKline Beecham, Philadelphia, Pa.); salts of calcium, iron or zinc; an insoluble suspension of acylated tyrosine; acylated sugars; cationically or anionically derivatized polysaccharides; polyphosphazenes; biodegradable microspheres;

monophosphoryl lipid A and Quil A. Cytokines, such as GM-CSF or interleukin-2, -7, or -12, may also be used as adjuvants. Hemocyanins and hemoerythrins may also be used in the invention. The use of hemocyanin from keyhole limpet (KLH) is particularly preferred, although other molluscan and arthropod hemocyanins and hemoerythrins may be employed. Various polysaccharide adjuvants may also be used. Polyamine varieties of polysaccharides are particularly preferred, such as chitin and chitosan, including deacetylated chitin.

A further preferred group of adjuvants are the muramyl dipeptide (MDP, N-acetylmuramyl-L-alanyl-D-isoglutamine) group of bacterial peptidoglycans. Derivatives of muramyl dipeptide, such as the amino acid derivative threonyl-MDP, and the fatty acid derivative MTPPE, are also contemplated.

U.S. Pat. No. 4,950,645 describes a lipophilic disaccharide-tripeptide derivative of muramyl dipeptide that is proposed for use in artificial liposomes formed from phosphatidyl choline and phosphatidyl glycerol. It is said to be effective in activating human monocytes and destroying tumor cells, but is non-toxic in generally high doses. The compounds of U. S. Pat. No. 4,950,645 and Intl. Pat. Appl. Publ. No. WO 91/16347, are also proposed for use in achieving particular aspects of the present invention.

BCG and BCG-cell wall skeleton (CWS) may also be used as adjuvants in the invention, with or without trehalose dimycolate. Trehalose dimycolate may be used itself. Azuma et al. (1988) show that trehalose dimycolate administration correlates with augmented resistance to influenza virus infection in mice. Trehalose dimycolate may be prepared as described in U.S. Pat. No. 4,579,945.

Amphipathic and surface-active agents, e.g., saponin and derivatives such as QS21 (Cambridge Biotech), form yet another group of preferred adjuvants for use with the immunogens of the present invention. Nonionic block copolymer surfactants (Rabinovich et al., 1994; Hunter et al., 1991) may also be employed. Oligonucleotides, as described by Yamamoto et al. (1988) are another useful group of adjuvants. Quil A and lentinen are also preferred adjuvants.

Superantigens are also contemplated for use as adjuvants in the present invention. “Superantigens” are generally bacterial products that stimulate a greater proportion of T lymphocytes than peptide antigens without a requirement for antigen processing (Mooney et. al., 1994). Superantigens include Staphylococcus exoproteins, such as the α, β, γ and δenterotoxins from S. aureus and S. epidermidis, and the α, β, γ and δ E. coli exotoxins.

Common Staphylococcus enterotoxins are known as staphylococcal enterotoxin A (SEA) and staphylococcal enterotoxin B (SEB), with enterotoxins through E (SEE) being described (Rott et. al., 1992). Streptococcus pyogenes B (SEB), Clostridium perftringens enterotoxin (Bowness et. al., 1992), cytoplasmic membrane-associated protein (CAP) from S. pyogenes (Sato et. al., 1994) and toxic shock syndrome toxin-1 (TSST-1) from S. aureus (Schwab et. al., 1993) are further useful superantigens.

One group of adjuvants particularly preferred for use in the invention are the detoxified endotoxins, such as the refined detoxified endotoxin of U.S. Pat. No. 4,866,034. These refined detoxified endotoxins are effective in producing adjuvant responses in mammals.

The detoxified endotoxins may be combined with other adjuvants. Combination of detoxified endotoxins with trehalose dimycolate is contemplated, as described in U.S. Pat. No. 4,435,386. Combinations of detoxified endotoxins with trehalose dimycolate and endotoxic glycolipids is also contemplated (U.S. Pat. No. 4,505,899), as is combination of detoxified endotoxins with cell wall skeleton (CWS) or CWS and trehalose dimycolate, as described in U.S. Pat. Nos. 4,436,727, 4,436,728 and 4,505,900. Combinations of just CWS and trehalose dimycolate, without detoxified endotoxins are also envisioned to be useful, as described in U.S. Pat. No. 4,520,019.

MPL is currently one preferred immunopotentiating agent for use herein. References that concern the uses of MPL include Tomai et al. (1987), Chen et al. (1991) and Garg and Subbarao (1992), that each concern certain roles of MPL in the reactions of aging mice; Elliott et al. (1991), that concerns the D-galactosamine loaded mouse and its enhanced sensitivity to lipopolysaccharide and MPL; Chase et al. (1986), that relates to bacterial infections; and Masihi et al. (1988), that describes the effects of MPL and endotoxin on resistance of mice to Toxoplasma gondii. Fitzgerald (1991) also reported on the use of MPL to up-regulate the immunogenicty of a syphilis vaccine and to confer significant protection against challenge infection in rabbits.

Thus MPL is known to be safe for use, as shown in the above model systems. Phase-I clinical trials have also shown MPL to be safe for use (Vosika et al., 1984). Indeed, 100 μg/m² is known to be safe for human use, even on an outpatient basis (Vosika et al, 1984).

MPL generally induces polyclonal B cell activation (Baker et al., 1994), and has been shown to augment antibody production in many systems, for example, in immunologically immature mice (Baker et al., 1988); in aging mice (Tomai and Johnson, 1989); and in nude and Xid mice (Madonna and Vogel, 1986; Myers et al., 1995). Antibody production has been shown against erythrocytes (Hraba et al, 1993); T cell dependent and independent antigens; Pnu-immune vaccine (Garg and Subbarao, 1992); isolated tumor-associated antigens (U.S. Pat. No. 4,877,611); against syngeneic tumor cells (Livingston etal., 1985; Ravindranath et al., 1994a;b); and against tumor-associated gangliosides (Ravindranath et al., 1994a;b).

Another useful attribute of MPL is that is augments IgM responses, as shown by Baker et al (1988a), who describe the ability of MPL to increase antibody responses in young mice. This is a particularly useful feature of an adjuvant for use in certain embodiments of the present invention. Myers et al. (1995) recently reported on the ability of MPL to induce IgM antibodies, by virtue T cell-independent antibody production.

In the Myers et al. (1995) studies, MPL was conjugated to the hapten, TNP. MPL was proposed for use as a carrier for other haptens, such as peptides.

MPL also activates and recruits macrophages (Verma et al., 1992). Tomai and Johnson (1989) showed that MPL-stimulated T cells enhance IL-1 secretion by macrophages. MPL is also known to activate superoxide production, lysozyme activity, phagocytosis, and killing of Candida in murine peritoneal macrophages (Chen et al., 1991).

The effects of MPL on T cells include the endogenous production of cytotoxic factors, such as TNF, in serum of BCG-primed mice by MPL (Bennett et al., 1988). Kovach etal. (1990) and Elliot etal. (1991) also show that MPL induces TNF activity. MPL is known to act with TNF-α to induce release of IFN-γ by NK cells. IFN-γ production by T cells in response to MPL was also documented by Tomai and Johnson (1989) and Odean etal. (1990).

MPL is also known to be a potent T cell adjuvant. For example, MPL stimulates proliferation of melanoma-antigen specific CTLs (Mitchell et al., 1988, 1993). Further, Baker et al. (1988b) showed that nontoxic MPL inactivated suppressor T cell activity. Naturally, in the physiological environment, the inactivation of T suppressor cells allows for increased benefit for the animal, as realized by, e.g., increased antibody production. Johnson and Tomai (1988) have reported on the possible cellular and molecular mediators of the adjuvant action of MPL.

MPL is also known to induce aggregation of platelets and to phosphorylate a platelet protein prior to induction of serotonin secretion (Grabarek et al., 1990). This study shows that MPL is involved in protein kinase C activation and signal transduction.

Many articles concern the structure and function of MPL include. These include Johnson et al. (1990), that describes the structural characterization of MPL homologs obtained from Salmonella Minnesota Re595 lipopolysaccharide. The work of Johnson et al. (1990), in common with Grabarek et al. (1990), shows that the fatty acid moieties of MPL can vary, even in commercial species. In separating MPL into eight fractions by thin layer chromatography, Johnson et al. (1990) found that three were particularly active, as assessed using human platelet responses. The chemical components of the various MPL species were characterized by Johnson et al. (1990).

Baker et al. (1992) further analyzed the structural features that influence the ability of lipid A and its analogs to abolish expression of suppressor T cell activity. They reported that decreasing the number of phosphate groups in lipid A from two to one (i.e., creating monophosphoryl lipid A, MPL) as well as decreasing the fatty acyl content, primarily by removing the residue at the 3 position, resulted in a progressive reduction in toxicity; however, these structural modifications did not influence its ability to abolish the expression of Ts function (Baker et al., 1992). These types of MPL are ideal for use in the present invention.

Baker et al. (1992) also showed that reducing the fatty acyl content from five to four (lipid A precursor IV_{A} or Iₐ) eliminated the capacity to influence Ts function but not to induce polyclonal activation of B cells. These studies show that in order to be able to abolish the expression of Ts function, lipid A must be a glucosamine disaccharide; may have either one or two phosphate groups; and must have at least five fatty acyl groups. Also, the chain length of the nonhydroxylated fatty acid, as well as the location of acyloxyacyl groups (2′ versus 3′ position), may play an important role (Baker et al., 1992).

In examining the relationship between chain length and position of fatty acyl groups on the ability of lipid A to abolish the expression of suppressor T-cell (Ts) activity, Baker et al. (1994) found that fatty acyl chain lengths of C₁₂ to C₁₄ appeared to be optimal for bioactivity. Therefore, although their use is still possible, lipid A preparations with fatty acyl groups of relatively short chain length (C₁₀ to C₁₂ from Pseudomonas aeruginosa and Chromobacterium violaceum) or predominantly long chain length (C₁₈ from Helicobacter pylori) are less preferred for use in this invention.

Baker et al. (1994) also showed that the lipid A proximal inner core region oligosaccharides of some bacterial lipopolysaccharides increase the expression of Ts activity; due mainly to the capacity of such oligosaccharides, which are relatively conserved in structure among gram-negative bacterial, to enlarge or expand upon the population of CD8+ Ts generated during the course of a normal antibody response to unrelated microbial antigens. The minimal structure required for the expression of the added immunosuppression observed was reported to be a hexasaccharide containing one 2-keto-3-deoxyoctonate residue, two glucose residues, and three heptose residues to which are attached two pyrophosphorylethanolamine groups (Baker et al., 1994). This information may be considered in utilizing or even designing further adjuvants for use in the invention.

In a generally related line of work, Tanamoto et al. (1994a;b; 1995) described the dissociation of endotoxic activities in a chemically synthesized Lipid A precursor after acetylation or succinylation. Thus, compounds such as “acetyl 406” and “succinyl 516” (Tanamoto et al., 1994a;b; 1995) are also contemplated for use in the invention.

Synthetic MPLs form a particularly preferred group of antigens. For example, Brade et al. (1993) described an artificial glycoconjugate containing the bisphosphorylated glucosamine disaccharide backbone of lipid A that binds to anti-Lipid A MAbs. This is one candidate for use in certain aspects of the invention.

The MPL derivatives described in U.S. Pat. No. 4,987,237 are particularly contemplated for use in the present invention. U.S. Pat. No. 4,987,237 describes MPL derivatives that contain one or more free groups, such as amines, on a side chain attached to the primary hydroxyl groups of the monophosphoryl lipid A nucleus through an ester group. The derivatives provide a convenient method for coupling the lipid A through coupling agents to various biologically active materials. The immunostimulant properties of lipid A are maintained. All MPL derivatives in accordance with U.S. Pat. No. 4,987,237 are envisioned for use in the MPL adjuvant-incorporated cells of this invention.

Various adjuvants, even those that are not commonly used in humans, may still be employed in animals, where, for example, one desires to raise antibodies or to subsequently obtain activated T cells. The toxicity or other adverse effects that may result from either the adjuvant or the cells, e.g., as may occur using non-irradiated tumor cells, is irrelevant in such circumstances.

Within the vaccines provided herein, the adjuvant composition is preferably designed to induce an immune response predominantly of the Thl type. High levels of Thl-type cytokines (e.g., IFN-γ, TNFα, IL-2 and IL-12) tend to favor the induction of cell-mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines see e.g., Mosmann and Coffman (1989).

Preferred adjuvants for use in eliciting a predominantly Th1-type response include, for example, a combination of monophosphoryl lipid A, preferably 3-de-O-acylated mono-phosphoryl lipid A (3D-MPL), together with an aluminum salt. MPL adjuvants are available from Corixa Corporation (Seattle, Wash.; see e.g., U.S. Pat. Nos. 4,436,727; 4,877,611; 4,866,034 and 4,912,094, each of which is specifically incorporated herein by reference in its entirety). CpG-containing oligonucleotides (in which the CpG dinucleotide is unmethylated) also induce a predominantly Thl response. Such oligonucleotides are well known and are described, for example, in Intl. Pat. Appl. Publ. No. WO 96/02555 and Intl. Pat. Appl. Publ. No. WO 99/33488. Immunostimulatory DNA sequences are also described, for example, by Sato et al. (1996). Another preferred adjuvant is a saponin, preferably QS21 (Aquila Biopharmaceuticals Inc., Framingham, Mass.), which may be used alone or in combination with other adjuvants. For example, an enhanced system involves the combination of a monophosphoryl lipid A and saponin derivative, such as the combination of QS21 and 3D-MPL (see e.g., Intl. Pat. Appl. Publ. No. WO 94/00153), or a less reactogenic composition where the QS21 is quenched with cholesterol (see e.g., Intl. Pat. Appl. Publ. No. WO 96/33739). Other preferred formulations comprise an oil-in-water emulsion and tocopherol. A particularly potent adjuvant formulation involving QS21, 3D-MPL and tocopherol in an oil-in-water emulsion has also been described (see e.g., Intl. Pat. Appl. Publ. No. WO 95/17210).

Other preferred adjuvants include Montanide ISA 720 (Seppic), SAF (Chiron), ISCOMS (CSL), MF-59 (Chiron), the SBAS series of adjuvants (e.g., SBAS-2 or SBAS-4, available from SmithKline Beecham, Rixensart, Belgium), Detox (Corixa Corporation), RC-529 (Corixa Corporation) and aminoalkyl glucosaminide 4-phosphates (AGPs).

Any vaccine provided herein may be prepared using well-known methods that result in a combination of one or more antigens, one or more immunostimulants or adjuvants and one or more suitable carriers, excipients, or pharmaceutically acceptable buffers. The compositions described herein may be administered as part of a sustained release formulation (i.e., a formulation such as a capsule, sponge or gel [composed of polysaccharides, for example] that effects a slow release of compound following administration). Such formulations may generally be prepared using well-known technology (Coombes et al., 1996) and administered by, for example, oral, rectal or subcutaneous implantation, or by implantation at the desired target site. Sustained-release formulations may contain a peptide, polynucleotide or antibody dispersed in a carrier matrix and/or contained within a reservoir surrounded by a rate-controlling membrane.

Carriers for use within such formulations are preferably biocompatible, and may also be biodegradable; preferably the formulation provides a relatively constant level of active component release. Such carriers include microparticles of poly(lactide-co-glycolide), as well as polyacrylate, latex, starch, cellulose and dextran. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (U.S. Pat. No. 5,151,254; Intl. Pat. Appl. Publ. No. WO 94/20078; Intl. Pat. Appl. Publ. No. WO/94/23701; and Intl. Pat. Appl. Publ. No. WO 96/06638). The amount of active compound contained within a sustained release formulation depends upon the site of implantation, the rate and expected duration of release and the nature of the condition to be treated or prevented.

Any of a variety of delivery vehicles may be employed within pharmaceutical compositions and vaccines to facilitate production of an antigen-specific immune response that targets tumor cells. Delivery vehicles include antigen-presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, 1998) and have been shown to be effective as a physiological adjuvant for eliciting prophylactic or therapeutic antitumor immunity (Timmerman and Levy, 1999). In general, dendritic cells may be identified based on their typical shape (stellate in situ, with marked cytoplasmic processes (dendrites) visible in vitro), their ability to take up, process and present antigens with high efficiency and their ability to activate naive T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (Zitvogel et al, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL- 13 and/or TNFA to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcy receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide encoding a WT1 peptide, such that the peptide, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen-presenting cell may be administered to a patient, resulting in transfection that occurs in vivo. In vivo and ex vivo transfection of dendritic cells, for example, may generally be performed using any methods known in the art, such as those described in Intl. Pat. Appl. Publ. No. WO 97/24447, or the gene gun approach described by Mahvi et al. (1997). Antigen loading of dendritic cells may be achieved by incubating dendritic cells or progenitor cells with the WT1 peptide, DNA (naked or within a plasmid vector) or RNA; or with antigen-expressing recombinant bacterium or viruses (e.g., vaccinia, fowlpox, adenovirus or lentivirus vectors). Prior to loading, the peptide may be covalently conjugated to an immunological partner that provides T cell help (e.g., a carrier molecule). Alternatively, a dendritic cell may be pulsed with a non-conjugated immunological partner, separately or in the presence of the peptide.

Combined therapeutics is also contemplated, and the same type of underlying pharmaceutical compositions may be employed for both single and combined medicaments. Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

4.8 Methods for the Therapy of Malignant Disease

In further aspects of the present invention, the compositions and vaccines described herein may be used to inhibit the development of malignant diseases (e.g., progressive or metastatic diseases or diseases characterized by small tumor burden such as minimal residual disease). In general, such methods may be used to prevent, delay or treat a disease associated with WT1 expression. In other words, therapeutic methods provided herein may be used to treat an existing WT1-associated disease, or may be used to prevent or delay the onset of such a disease in a patient who is free of disease or who is afflicted with a disease that is not yet associated with WT1 expression.

As used herein, a disease is “associated with WT1 expression” if diseased cells (e.g., tumor cells) at some time during the course of the disease generate detectably higher levels of a WT1 peptide than normal cells of the same tissue. Association of WT1 expression with a malignant disease does not require that WT1 be present on a tumor. For example, overexpression of WT1 may be involved with initiation of a tumor, but the protein expression may subsequently be lost. Alternatively, a malignant disease that is not characterized by an increase in WT1 expression may, at a later time, progress to a disease that is characterized by increased WT1 expression. Accordingly, any malignant disease in which diseased cells formerly expressed, currently express or are expected to subsequently express increased levels of WT1 is considered to be “associated with WT1 expression.” Within certain embodiments, the therapies provided herein are administered to a patient afflicted with, or considered at risk for, malignant mesothelioma.

Immunotherapy may be performed using any of a variety of techniques, in which compounds or cells provided herein function to remove WT1 -expressing cells from a patient. Such removal may take place as a result of enhancing or inducing an immune response in a patient specific for WT1 or a cell expressing WT1. Alternatively, WT1-expressing cells may be removed ex vivo (e.g., by treatment of autologous bone marrow, peripheral blood or a fraction of bone marrow or peripheral blood). Fractions of bone marrow or peripheral blood may be obtained using any standard technique in the art.

Within such methods, pharmaceutical compositions and vaccines may be administered to a patient. As used herein, a “patient” refers to any warm-blooded animal, preferably a human. A patient may or may not be afflicted with a malignant disease. Accordingly, the above pharmaceutical compositions and vaccines may be used to prevent the onset of a disease (i.e., prophylactically) or to treat a patient afflicted with a disease (e.g., to prevent or delay progression and/or metastasis of an existing disease). A patient afflicted with a disease may have a minimal residual disease (e.g., a low tumor burden in a leukemia patient in complete or partial remission or a cancer patient following reduction of the tumor burden after surgery radiotherapy and/or chemotherapy). Such a patient may be immunized to inhibit a relapse (i.e., prevent or delay the relapse, or decrease the severity of a relapse). Within certain preferred embodiments, the patient is afflicted with malignant mesothelioma. Other WT1-associated conditions include leukemia (e.g., AML, CML, ALL or childhood ALL), a myelodysplastic syndrome (MDS) and cancer (e.g., gastrointestinal, lung, thyroid or breast cancer or a melanoma), where the cancer or leukemia is WT1 positive (i.e., reacts detectably with an anti-WT1 antibody, as provided herein or expresses WT1 mRNA at a level detectable by RT-PCR™, as described herein), as well as autoimmune diseases directed against WT1 -expressing cells.

Other diseases associated with WT1 overexpression include kidney cancer (such as renal cell carcinoma, or Wilms tumor), as described in Satoh et al. (2000), and Campbell et al. (1998); and mesothelioma, as described in Amin et al., (1995). Harada et al. (1999) describe WT1 gene expression in human testicular germ-cell tumors. Nonomura et al. Hinyokika (1999) describe molecular staging of testicular cancer using polymerase chain reaction of the testicular cancer-specific genes. Shimizu et al. (2000) describe the immunohistochemical detection of the Wilms' tumor gene (WT1) in epithelial ovarian tumors.

WT1 overexpression was also described in desmoplastic small round cell tumors, by Bamoud, et al., (2000). WT1 overexpression in glioblastoma and other cancer was described by Menssen et al., (2000), “Wilms' tumor gene (WT1) expression in lung cancer, colon cancer and glioblastoma cell lines compared to freshly isolated tumor specimens.” Other diseases showing WT1 overexpression include EBV associated diseases, such as Burkitt's lymphoma and nasopharyngeal cancer (Spinsanti et al., 2000), “Wilms' tumor gene expression by normal and malignant human B lymphocytes.”

Pan et al. (2000) describe in vitro IL-12 treatment of peripheral blood mononuclear cells from patients with leukemia or myelodysplastic syndromes, and reported an increase in cytotoxicity and reduction in WT1 gene expression. Patmasiriwat et al. (1999) reported WT1 and GATA1 expression in myelodysplastic syndrome and acute leukemia. Tamaki et al. (1999) reported that the Wilms tumor gene WT1 is a good marker for diagnosis of disease progression of myelodysplastic syndromes. Expression of the Wilms tumor gene WT1 in solid tumors, and its involvement in tumor cell growth, was discussed in relation to gastric cancer, colon cancer, lung cancer, breast cancer cell lines, germ cell tumor cell line, ovarian cancer, the uterine cancer, thyroid cancer cell line, hepatocellular carcinoma, in Oji et al. (1999).

The compositions provided herein may be used alone or in combination with conventional therapeutic regimens such as surgery, irradiation, chemotherapy and/or bone marrow transplantation (autologous, syngeneic, allogeneic or unrelated). As discussed in greater detail below, binding agents and T cells as provided herein may be used for purging of autologous stem cells. Such purging may be beneficial prior to, for example, bone marrow transplantation or transfusion of blood or components thereof. Binding agents, T cells, antigen-presenting cells (APC) and compositions provided herein may further be used for expanding and stimulating (or priming) autologous, allogeneic, syngeneic or unrelated WT1-specific T cells in vitro and/or in vivo. Such WT1-specific T cells may be used, for example, within donor lymphocyte infusions.

Routes and frequency of administration, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. In some tumors, pharmaceutical compositions or vaccines may be administered locally (by, for example, rectocoloscopy, gastroscopy, videoendoscopy, angiography or other methods known in the art). Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response that is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent complete or partial remissions, or longer disease-free and/or overall survival) in vaccinated patients as compared to non-vaccinated patients. In general, for pharmaceutical compositions and vaccines comprising one or more peptides, the amount of each peptide present in a dose ranges from about 100 μg to 5 mg. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent complete or partial remissions, or longer disease-free and/or overall survival) in treated patients as compared to non-treated patients. Increases in preexisting immune responses to WT1 generally correlate with an improved clinical outcome. Such immune responses may generally be evaluated using standard proliferation, cytotoxicity or cytokine assays, which may be performed using samples obtained from a patient before and after treatment.

Within further aspects, methods for inhibiting the development of a malignant disease associated with WT1 expression involve the administration of autologous T cells that have been activated in response to a WT1 peptide or WT1-expressing APC, as described above. Such T cells may be CD4⁺ and/or CD8⁺, and may be proliferated as described above. The T cells may be administered to the individual in an amount effective to inhibit the development of a malignant disease. Typically, about 1×10⁹ to 1×10¹¹ T cells/M² are administered intravenously, intracavitary or in the bed of a resected tumor. It will be evident to those skilled in the art that the number of cells and the frequency of administration will be dependent upon the response of the patient.

Within certain embodiments, T cells may be stimulated prior to an autologous bone marrow transplantation. Such stimulation may take place in vivo or in vitro. For in vitro stimulation, bone marrow and/or peripheral blood (or a fraction of bone marrow or peripheral blood) obtained from a patient may be contacted with a WT1 peptide, a polynucleotide encoding a WT1 peptide and/or an APC that expresses a WT1 peptide under conditions and for a time sufficient to permit the stimulation of T cells as described above. Bone marrow, peripheral blood stem cells and/or WT1-specific T cells may then be administered to a patient using standard techniques.

Within related embodiments, T cells of a related or unrelated donor may be stimulated prior to a syngeneic, or allogeneic (related or unrelated), bone marrow transplantation. Such stimulation may take place in vivo or in vitro. For in vitro stimulation, bone marrow and/or peripheral blood (or a fraction of bone marrow or peripheral blood) obtained from a related or unrelated donor may be contacted with a WT1 peptide, WT1 polynucleotide and/or APC that expresses a WT1 peptide under conditions and for a time sufficient to permit the stimulation of T cells as described above. Bone marrow, peripheral blood stem cells and/or WT1-specific T cells may then be administered to a patient using standard techniques.

Within other embodiments, WT1-specific T cells as described herein may be used to remove cells expressing WT1 from autologous bone marrow, peripheral blood or a fraction of bone marrow or peripheral blood (e.g., CD34⁺ enriched peripheral blood (PB) prior to administration to a patient). Such methods may be performed by contacting bone marrow or PB with such T cells under conditions and for a time sufficient to permit the reduction of WT1-expressing cells to less than 10%, preferably less than 5% and more preferably less than 1%, of the total number of myeloid or lymphatic cells in the bone marrow or peripheral blood. The extent to which such cells have been removed may be readily determined by standard methods such as, for example, qualitative and quantitative PCR™ analysis, morphology, immunohistochemistry and FACS analysis. Bone marrow or PB (or a fraction thereof) may then be administered to a patient using standard techniques.

4.9 Diagnostic and Prognostic Methods for WT1 -Specific Disease

The present invention further provides methods for detecting a malignant disease associated with WT1 expression, and for monitoring the effectiveness of an immunization or therapy for such a disease. Such methods are based on the discovery, within the present invention, that an immune response specific for WT1 protein can be detected in patients afflicted with such diseases, including malignant mesothelioma, and that methods which enhance such immune responses may provide a preventive or therapeutic benefit. Diagnostic methods provided herein may provide early detection of these diseases, and permit the high throughput screening of patients considered at risk. Such patients include, for example, individuals suspected of asbestos exposure, which may be at risk for the development of malignant mesothelioma.

To determine the presence or absence of a malignant disease associated with WT1 expression, a patient may be tested for the level of T cells specific for WT1. Within certain methods, a biological sample comprising CD4⁺ and/or CD8⁺ T cells isolated from a patient is incubated with a WT1 peptide, a polynucleotide encoding a WT1 peptide and/or an APC that expresses a WT1 peptide, and the presence or absence of specific activation of the T cells is detected, as described herein. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with WT1 peptide (e.g., 5-25 μg/ml). It may be desirable to incubate another aliquot of a T cell sample in the absence of WT1 peptide to serve as a control. For CD4⁺ T cells, activation is preferably detected by evaluating proliferation of the T cells. For CD8⁺ T cells, activation is preferably detected by evaluating cytolytic activity. A level of proliferation that is at least two fold greater and/or a level of cytolytic activity that is at least 20% greater than in disease-free patients indicates the presence of a malignant disease associated with WT1 expression. Further correlation may be made, using methods well known in the art, between the level of proliferation and/or cytolytic activity and the predicted response to therapy. In particular, patients that display a higher antibody, proliferative and/or lytic response may be expected to show a greater response to therapy.

Within other methods, a biological sample obtained from a patient is tested for the level of antibody specific for WT1. The biological sample is incubated with a WT1 peptide, a polynucleotide encoding a WT1 peptide and/or an APC that expresses a WT1 peptide under conditions and for a time sufficient to allow immunocomplexes to form. Immunocomplexes formed between the WT1 peptide and antibodies in the biological sample that specifically bind to the WT1 peptide are then detected. A biological sample for use within such methods may be any sample obtained from a patient that would be expected to contain antibodies. Suitable biological samples include blood, sera, ascites, bone marrow, pleural effusion, and cerebrospinal fluid.

The biological sample is incubated with the WT1 peptide in a reaction mixture under conditions and for a time sufficient to permit immunocomplexes to form between the peptide and antibodies specific for WT1. For example, a biological sample and WT1 peptide may be incubated at 4° C. for 24-48 hrs.

Following the incubation, the reaction mixture is tested for the presence of immunocomplexes. Detection of immunocomplexes formed between the WT1 peptide and antibodies present in the biological sample may be accomplished by a variety of known techniques, such as radioimmunoassays (RIA) and enzyme linked immunosorbent assays (ELISA). Suitable assays are well known in the art and are amply described in the scientific and patent literature (Harlow and Lane, 1988). Assays that may be used include, but are not limited to, the double monoclonal antibody sandwich immunoassay technique (U.S. Pat. No. 4,376,110); monoclonal-polyclonal antibody sandwich assays (Wide et al., 1970); the “western blot” method (U.S. Pat. No. 4,452,901); immunoprecipitation of labeled ligand (Brown et al., 1980); enzyme-linked immunosorbent assays (Raines and Ross, 1982); immunocytochemical techniques, including the use of fluorochromes (Brooks et al., 1980); and neutralization of activity (Bowen-Pope et al., 1984). Other immunoassays include, but are not limited to, those described in U.S. Pat. Nos.: 3,817,827; 3,850,752; 3,901,654; 3,935,074; 3,984,533; 3,996,345; 4,034,074; and 4,098,876.

For detection purposes, WT1 peptide may either be labeled or unlabeled. Unlabeled WT1 peptide may be used in agglutination assays or in combination with labeled detection reagents that bind to the immunocomplexes (e.g., anti-immunoglobulin, protein G, Protein A or a lectin and secondary antibodies, or antigen-binding fragments thereof, capable of binding to the antibodies that specifically bind to the WT1 peptide). If the WT1 peptide is labeled, the reporter group may be any suitable reporter group known in the art, including radioisotopes, fluorescent groups, luminescent groups, enzymes, biotin and dye particles.

Within certain assays, unlabeled WT1 peptide is immobilized on a solid support. The solid support may be any material known to those of ordinary skill in the art to which the peptide may be attached. For example, the solid support may be a test well in a microtiter plate or a nitrocellulose or other suitable membrane. Alternatively, the support may be a bead or disc, such as glass, fiberglass, latex or a plastic material such as polystyrene or poly-vinylchloride. The support may also be a magnetic particle or a fiber optic sensor, such as those disclosed, for example, in U.S. Pat. No. 5,359,681. The peptide may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the antigen and functional groups on the support or may be a linkage by way of a cross-linking agent). Immobilization by adsorption to a well in a microtiter plate or to a membrane is preferred. In such cases, adsorption may be achieved by contacting the WT1 peptide, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of peptide ranging from about 10 ng to about 10 μg, and preferably about 100 ng to about 1 μg, is sufficient to immobilize an adequate amount of peptide.

Following immobilization, the remaining protein binding sites on the support are typically blocked. Any suitable blocking agent known to those of ordinary skill in the art, such as bovine serum albumin, Tween™ 20™ (Sigma Chemical Co., St. Louis, MO), heat-inactivated normal goat serum (NGS), or BLOTTO (buffered solution of nonfat dry milk which also contains a preservative, salts, and an antifoaming agent) may be used. The support is then incubated with a biological sample suspected of containing specific antibody. The sample can be applied neat, or, more often, it can be diluted, usually in a buffered solution which contains a small amount (0.1%-5.0% by weight) of protein, such as BSA, NGS, or BLOTTO. In general, an appropriate contact time (i.e., incubation time) is a period of time that is sufficient to detect the presence of antibody that specifically binds WT1 within a sample containing such an antibody. Preferably, the contact time is sufficient to achieve a level of binding that is at least about 95% of that achieved at equilibrium between bound and unbound antibody. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% TweenTm 20. A detection reagent that binds to the immunocomplexes and that comprises a reporter group may then be added. The detection reagent is incubated with the immunocomplex for an amount of time sufficient to detect the bound antibody. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups (e.g., horseradish peroxidase, beta-galactosidase, alkaline phosphatase and glucose oxidase) may generally be detected by the addition of substrate (generally for a specific period of time), followed by spectroscopic or other analysis of the reaction products. Regardless of the specific method employed, a level of bound detection reagent that is at least two fold greater than background (i.e., the level observed for a biological sample obtained from a disease-free individual) indicates the presence of a malignant disease associated with WT1 expression.

In general, methods for monitoring the effectiveness of an immunization or therapy involve monitoring changes in the level of antibodies or T cells specific for WT1 in the patient. Methods in which antibody levels are monitored may comprise the steps of: (a) incubating a first biological sample, obtained from a patient prior to a therapy or immunization, with a WT1 peptide, wherein the incubation is performed under conditions and for a time sufficient to allow immunocomplexes to form; (b) detecting immunocomplexes formed between the WT1 peptide and antibodies in the biological sample that specifically bind to the WT1 peptide; (c) repeating steps (a) and (b) using a second biological sample taken from the patient following therapy or immunization; and (d) comparing the number of immunocomplexes detected in the first and second biological samples. Alternatively, a polynucleotide encoding a WT1 peptide, or an APC expressing a WT1 peptide may be employed in place of the WT1 peptide. Within such methods, immunocomplexes between the WT1 peptide encoded by the polynucleotide, or expressed by the APC, and antibodies in the biological sample are detected.

Methods in which T cell activation and/or the number of WT1 specific precursors are monitored may comprise the steps of: (a) incubating a first biological sample comprising CD4⁺ and/or CD8⁺ cells (e.g., bone marrow, peripheral blood or a fraction thereof), obtained from a patient prior to a therapy or immunization, with a WT1 peptide, wherein the incubation is performed under conditions and for a time sufficient to allow specific activation, proliferation and/or lysis of T cells; (b) detecting an amount of activation, proliferation and/or lysis of the T cells; (c) repeating steps (a) and (b) using a second biological sample comprising CD4⁺ and/or CD8⁺ T cells, and taken from the same patient following therapy or immunization; and (d) comparing the amount of activation, proliferation and/or lysis of T cells in the first and second biological samples. Alternatively, a polynucleotide encoding a WT1 peptide, or an APC expressing a WT1 peptide may be employed in place of the WT1 peptide.

A biological sample for use within such methods may be any sample obtained from a patient that would be expected to contain antibodies, CD4⁺ T cells and/or CD8⁺ T cells. Suitable biological samples include blood, sera, ascites, bone marrow, pleural effusion and cerebrospinal fluid. A first biological sample may be obtained prior to initiation of therapy or immunization or part way through a therapy or vaccination regime. The second biological sample should be obtained in a similar manner, but at a time following additional therapy or immunization. The second biological sample may be obtained at the completion of, or part way through, therapy or immunization, provided that at least a portion of therapy or immunization takes place between the isolation of the first and second biological samples.

Incubation and detection steps for both samples may generally be performed as described above. A statistically significant increase in the number of immunocomplexes in the second sample relative to the first sample reflects successful therapy or immunization.

4.10 Administration of Pharmaceutical Compositions and Formulations

In certain embodiments, the present invention concerns formulation of one or more of the polynucleotide compositions disclosed herein in pharmaceutically acceptable solutions for administration to a cell or an animal, either alone, or in combination with one or more other modalities of anti-cancer therapy.

It will also be understood that, if desired, the nucleic acid segment, RNA, or DNA compositions disclosed herein may be administered in combination with other agents as well, such as, e.g., proteins or peptides or various pharmaceutically-active agents. As long as the composition comprises at least one of the genetic expression constructs disclosed herein, there is virtually no limit to other components that may also be included, given that the additional agents do not cause a significant adverse effect upon contact with the target cells or host tissues. The RNA- or DNA-derived compositions may thus be delivered along with various other agents as required in the particular instance. Such RNA or DNA compositions may be purified from host cells or other biological sources, or alternatively may be chemically synthesized as described herein. Likewise, such compositions may comprise substituted or derivatized RNA or DNA compositions. Such compositions may include one or more therapeutic gene constructs, either alone, or in combination with one or more modified peptide or nucleic acid substituent derivatives, and/or other anticancer therapeutics.

The formulation of pharmaceutically-acceptable excipients and carrier solutions are well-known to those of skill in the art, as is the development of suitable dosing and treatment regimens for using the particular compositions described herein in a variety of treatment regimens, including e.g., oral, intravenous, intranasal, transdermal, intraprostatic, intratumoral, and/or intramuscular administration and formulation.

4.10.1 Injectable Delivery

For example, the pharmaceutical compositions disclosed herein may be administered parenterally, intravenously, intramuscularly, or even intraperitoneally as described in U.S. Pat. No. 5,543,158, U.S. Pat. No. 5,641,515 and U.S. Pat. No. 5,399,363 (each specifically incorporated herein by reference in its entirety). Solutions of the active compounds as free-base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, Hoover, 1975). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

Sterile injectable solutions may be prepared by incorporating the gene therapy constructs in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antiflngal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

4.10.2 Intranasal Delivery

One may use nasal solutions or sprays, aerosols or even inhalants for the treatment of mesothelioma with one of more of the disclosed peptides and polynucleotides. Nasal solutions are usually aqueous solutions designed to be administered to the nasal passages in drops or sprays. Nasal solutions are prepared so that they are similar in many respects to nasal secretions, so that normal ciliary action is maintained. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known.

Inhalations and inhalants are pharmaceutical preparations designed for delivering a drug or compound into the respiratory tree of a patient. A vapor or mist is administered and reaches the affected area, often to give relief from symptoms of bronchial and nasal congestion. However, this route can also be employed to deliver agents into the systemic circulation. Inhalations may be administered by the nasal or oral respiratory routes. The administration of inhalation solutions is only effective if the droplets are sufficiently fine and uniform in size so that the mist reaches the bronchioles.

Another group of products, also known as inhalations, and sometimes called insufflations, consists of finely powdered or liquid drugs that are carried into the respiratory passages by the use of special delivery systems, such as pharmaceutical aerosols, that hold a solution or suspension of the drug in a liquefied gas propellant. When released through a suitable valve and oral adapter, a metered does of the inhalation is propelled into the respiratory tract of the patient.

Particle size is of importance in the administration of this type of preparation. It has been reported that the optimum particle size for penetration into the pulmonary cavity is of the order of 0.5 to 7 μm. Fine mists are produced by pressurized aerosols and hence their use in considered advantageous.

4.10.3 Liposome-, Nanocapsule-, and Microparticle-mediated Delivery

In certain embodiments, the inventors contemplate the use of liposomes, nanocapsules, microparticles, microspheres, lipid particles, vesicles, and the like, for the introduction of the polynucleotide compositions of the present invention into suitable host cells. In particular, the polynucleotide compositions of the present invention may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids disclosed herein. The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-lives (Gabizon and Papahadjopoulos, 1988; Allen and Choun, 1987; U.S. Pat. No. 5,741,516, specifically incorporated herein by reference in its entirety). Further, various methods of liposome and liposome like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally resistant to transfection by other procedures including T cell suspensions, primary hepatocyte cultures and PC12 cells (Renneisen et al., 1990; Muller et al., 1990). In addition, liposomes are free of the DNA length constraints that are typical of viral-based delivery systems. Liposomes have been used effectively to introduce genes, drugs (Heath and Martin, 1986; Heath et al., 1986; Balazsovits et al., 1989; Fresta and Puglisi, 1996), radiotherapeutic agents (Pikul et al., 1987), enzymes (Imaizumi et al., 1990a; Imaizumi et al., 1990b), viruses (Faller and Baltimore, 1984), transcription factors and allosteric effectors (Nicolau and Gersonde, 1979) into a variety of cultured cell lines and animals. In addition, several successful clinical trails examining the effectiveness of liposome-mediated drug delivery have been completed (Lopez-Berestein et al., 1985a; 1985b; Coune, 1988; Sculier et al., 1988). Furthermore, several studies suggest that the use of liposomes is not associated with autoimmune responses, toxicity or gonadal localization after systemic delivery (Mori and Fukatsu, 1992).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 Mm. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e. in the aqueous spaces and within the bilayer itself, respectively. It is possible that the drug-bearing liposomes may even be employed for site-specific delivery of active agents by selectively modifying the liposomal formulation.

In addition to the teachings of Couvreur et al. (1977; 1988), the following information may be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars, and drugs.

Alternatively, the invention provides for pharmaceutically acceptable nanocapsule formulations of the polynucleotide compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made, as described (Couvreur et al., 1980; 1988; zur Muhlen et al., 1998; Zambaux et al. 1998; Pinto-Alphandry et al., 1995 and U.S. Pat. No. 5,145,684, specifically incorporated herein by reference in its entirety). In particular, methods of polynucleotide polynucleotide delivery to a target cell using either nanoparticles or nanospheres (Schwab et al., 1994; Truong-Le et al., 1998) are also particularly contemplated to be useful in formulating the disclosed compositions for administration to an animal, and to a human in particular.

4.11 Therapeutic AGENTS and Kits

The invention also provides one or more of the WT1 -specific antibodies or antigen binding fragments, or WT1-derived peptides or peptide variants formulated with one or more pharmaceutically acceptable excipients, carriers, diluents, adjuvants, and/or other components for administration to an animal in need thereof. In addition to the disclosed epitopes, antibodies and antigen binding fragments, antibody- or antigen binding fragment-encoding polynucleotides or additional anticancer agents, polynucleotides, peptides, antigens, or other therapeutic compounds as may be employed in the formulation of particular compositions and formulations disclosed herein, and particularly in the preparation of anticancer agents or anti-mesothelioma therapies for administration to the affected mammal.

As such, preferred animals for administration of the pharmaceutical compositions disclosed herein include mammals, and particularly humans. Other preferred animals include primates, sheep, goats, bovines, equines, porcines, lupines, canines, and felines, as well as any other mammalian species commonly considered pets, livestock, or commercially relevant animal species. The compositions and formulations may include partially or significantly purified polypeptide, polynucleotide, or antibody or antigen binding fragment compositions, either alone, or in combination with one or more additional active ingredients, anticancer agents, vaccines, adjuvants, or other therapeutics which may be obtained from natural or recombinant sources, or which may be obtainable naturally or either chemically synthesized, or alternatively produced in vitro from recombinant host cells expressing one or more nucleic acid segments that encode one or more such additional active ingredients, carriers, adjuvants, cofactors, or other therapeutic compound.

4.12 Diagnostic Reagents and Kits

The invention further provides diagnostic reagents and kits comprising one or more such reagents for use in a variety of diagnostic assays, including for example, immunoassays such as ELISA and “sandwich”-type immunoassays. Such kits may preferably include at least a first peptide, or a first antibody or antigen binding fragment of the invention, a functional fragment thereof, or a cocktail thereof, and means for signal generation. The kit's components may be pre-attached to a solid support, or may be applied to the surface of a solid support when the kit is used. The signal generating means may come pre-associated with an antibody of the invention or may require combination with one or more components, e.g., buffers, antibody-enzyme conjugates, enzyme substrates, or the like, prior to use. Kits may also include additional reagents, e.g., blocking reagents for reducing nonspecific binding to the solid phase surface, washing reagents, enzyme substrates, and the like. The solid phase surface may be in the form of microtiter plates, microspheres, or other materials suitable for immobilizing proteins, peptides, or polypeptides. Preferably, an enzyme that catalyzes the formation of a chemiluminescent or chromogenic product or the reduction of a chemiluminescent or chromogenic substrate is a component of the signal generating means. Such enzymes are well known in the art.

Such kits are useful in the detection, monitoring and diagnosis of conditions characterized by over-expression or inappropriate expression of WT1, or WT1-derived peptides and/or polypeptides.

The therapeutic and diagnostic kits of the present invention may also be prepared that comprise at least one of the antibody, peptide, antigen binding fragment, hybridoma, vector, vaccine, polynucleotide, or cellular compositions disclosed herein and instructions for using the composition as a diagnostic reagent or therapeutic agent. Containers for use in such kits may typically comprise at least one vial, test tube, flask, bottle, syringe or other suitable container, into which one or more of the diagnostic and/or therapeutic composition(s) may be placed, and preferably suitably aliquoted. Where a second therapeutic agent is also provided, the kit may also contain a second distinct container into which this second diagnostic and/or therapeutic composition may be placed. Alternatively, a plurality of compounds may be prepared in a single pharmaceutical composition, and may be packaged in a single container means, such as a vial, flask, syringe, bottle, or other suitable single container. The kits of the present invention will also typically include a means for containing the vial(s) in close confinement for commercial sale, such as, e.g., injection or blow-molded plastic containers into which the desired vial(s) are retained. Where a radiolabel, chromogenic, fluorigenic, or other type of detectable label or detecting means is included within the kit, the labeling agent may be provided either in the same container as the diagnostic or therapeutic composition itself, or may alternatively be placed in a second distinct container means into which this second composition may be placed and suitably aliquoted. Alternatively, the detection reagent and the label may be prepared in a single container means, and in most cases, the kit will also typically include a means for containing the vial(s) in close confinement for commercial sale and/or convenient packaging and delivery.

4.13 Exemplary Definitions

In accordance with the present invention, nucleic acid sequences include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from native sources, chemically synthesized, modified, or otherwise prepared in whole or in part by the hand of man.

Unless defined otherwise, all technical and scientific terms used herein have the same meaning as commonly understood by one of ordinary skill in the art to which this invention belongs. Although any methods and compositions similar or equivalent to those described herein can be used in the practice or testing of the present invention, the preferred methods and compositions are described herein. For purposes of the present invention, the following terms are defined below:

A, an: In accordance with long standing patent law convention, the words “a” and “an” when used in this application, including the claims, denotes “one or more”.

Expression: The combination of intracellular processes, including transcription and translation undergone by a polynucleotide such as a structural gene to synthesize the encoded peptide or polypeptide.

Promoter: a term used to generally describe the region or regions of a nucleic acid sequence that regulates transcription.

Regulatory Element: a term used to generally describe the region or regions of a nucleic acid sequence that regulates transcription.

Structural gene: A gene or sequence region that is expressed to produce an encoded peptide or polypeptide.

Transformation: A process of introducing an exogenous polynucleotide sequence (e.g., a vector, a recombinant DNA or RNA molecule) into a host cell or protoplast in which that exogenous nucleic acid segment is incorporated into at least a first chromosome or is capable of autonomous replication within the transformed host cell. Transfection, electroporation, and naked nucleic acid uptake all represent examples of techniques used to transform a host cell with one or more polynucleotides.

Transformed cell: A host cell whose nucleic acid complement has been altered by the introduction of one or more exogenous polynucleotides into that cell.

Transgenic cell: Any cell derived or regenerated from a transformed cell or derived from a transgenic cell, or from the progeny or offspring of any generation of such a transformed host cell.

Transgenic animal: An animal or a progeny or an offspring of any generation thereof that is derived from a transformed animal cell, wherein the animal's DNA contains an introduced exogenous nucleic acid molecule not originally present in a native, wild type, non-transgenic animal of the same species. The terms “transgenic animal” and “transformed animal” have sometimes been used in the art as synonymous terms to define an animal, the genetic contents of which has been modified to contain one or more exogenous nucleic acid segments.

Vector: A nucleic acid molecule, typically comprised of DNA, capable of replication in a host cell and/or to which another nucleic acid segment can be operatively linked so as to bring about replication of the attached segment. A plasmid, cosmid, or a virus is an exemplary vector.

The terms “substantially corresponds to”, “substantially homologous”, or “substantial identity” as used herein denotes a characteristic of a nucleic acid or an amino acid sequence, wherein a selected nucleic acid or amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared. The percentage of sequence identity may be calculated over the entire length of the sequences to be compared, or may be calculated by excluding small deletions or additions which total less than about 25 percent or so of the chosen reference sequence. The reference sequence may be a subset of a larger sequence, such as a portion of a gene or flanking sequence, or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides. Desirably, which highly homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as e.g., the FASTA program analysis described by Pearson and Lipman (1988).

The term “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally occurring animals.

As used herein, a “heterologous” is defined in relation to a predetermined referenced gene sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements. “Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

As used herein, a “transcription factor recognition site” and a “transcription factor binding site” refer to a polynucleotide sequence(s) or sequence motif(s) which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted on the basis of known consensus sequence motifs, or by other methods known to those of skill in the art.

As used herein, the term “operably linked” refers to a linkage of two or more polynucleotides or two or more nucleic acid sequences in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis sequences that are necessary for efficient transcription and translation (e.g., polyadenylation site(s), mRNA stability controlling sequence(s), etc.

### 5. EXAMPLES

The following examples are included to demonstrate preferred embodiments of the invention. However, those of skill in the art should, in light of the present disclosure, appreciate that many changes can be made in the specific embodiments which are disclosed and still obtain a like or similar result without departing from the spirit and scope of the invention described in the appended claims.

### 5.1 Example 1

### Detection of WT1 specific antibodies in Malignant Mesothielioma Patients

This example illustrates the use of WT1 as a marker for malignant mesothelioma.

5.1.1 Materials and Methods

5.1.1.1 Recombinant Protein Purification

For protein expression, CDNA constructs representing the human WT1 full length (amino acids 1-449), the N-terminus (amino acids 1-249) and C-terminus (amino acids 267-449) regions were subcloned into a modified pET28 vector. The resulting vector had a 5′ His tag, followed by the thioredoxin (Trx) coding region, followed by a 3′ Histidine tag, followed by a Thrombin and EK site. Recombinant BL21 pLysS E. coli (Stratagene, La Jolla, CA) containing the expression constructs for the TRX-WT1 Protein And the fusion proteins of truncated forms of WT1 with TRX were grown overnight and induced with isopropyl-β-D-thiogalactoside (IPTG). All the proteins behaved similarly and were purified following essentially the same protocol. Cells were harvested and lysed by incubation in 10 mM Tris pH=8.0 with complete protease inhibitor tablets (Boehringer Mannheim Biochemicals, Indianapolis, Ind.) at 37° C. followed by repeated rounds of sonication. Inclusion bodies obtained were washed twice with 10 mM Tris pH=8.0 / 0.5% CHAPS and solubilized in 8M urea containing 10 mM Tris at pH=8.0 (Buffer A). Proteins were then purified by metal chelate affinity chromatography over nickel nitrilotriacetic acid resin (QIAGEN Inc., Valencia, Calif.). Proteins were analyzed by SDS-PAGE and fractions containing the protein of interest were pooled and dialyzed overnight against excess 10 mM Tris pH=8.0. Dialysates were brought to 8 M urea and loaded onto a Source QTM anion exchange resin (Amersham Pharmacia Biotech, Uppsala, Sweden) equilibrated in buffer A. Proteins were eluted in buffer A with a gradient from 0 to 1 M NaCl. Fractions containing the proteins of interest were pooled, dialyzed overnight against excess 10 mM Tris pH=8.0, and stored at −80° C. for further use. The identity of the WT1 peptides was confirmed by N-terminal sequencing.

5.1.1.2 Antibody Responses

Antibody responses to WT1 polypeptides were determined by Western blot analysis using both recombinant full-length and truncated WT1 proteins and a WT1 polypeptide designated WT N180 (Santa Cruz Biotechnology, Inc., Santa Cruz, Calif.). As the primary antibody, sera from immunized as well as non-immunized B6 mice or human AML patients was used in a 1:500 dilution with Tris-buffered saline/1% BSA and 0.1% NP-40™. A polygonal antimouse or antihuman-horseradish peroxidase-conjugated second antibody (Amersham Pharmacia Biotech, Piscataway, NJ) was used in a 1:10,000 dilution. The blots were then developed by using a chemiluminescent reaction (ECL™ Reagent, Amersham) after which they were exposed to Hyperfilm-ECL™ (Amersham). The film was developed and examined. All control blots were developed by using the commercially prepared WT1-specific antibodies, WT C-19 and WT 180 (Santa Cruz Biotechnology), and each demonstrated a strong band at the expected size of the TRX-WT1 fusion proteins (TRX-WT1 full-length (approximately 85 kDa); TRX-WT1 N-terminus (approximately 60 kDa); TRX-WT1 C-terminus (approximately 50 kDa).

5.1.1.3 WT1 Elisa

96-well ELISA plates (Nunc) were coated with 50 μl/well of each of the WT1, proteins. WT1 proteins were diluted to 5 ng/μl in ELISA coating buffer (1 M Na₂HCO₃, pH 9.6). Plates were incubated over night at 4° C. or 4 hrs at 37° C. and then washed twice in PBS/0.1% Tween™. Plates were blocked with 200 μl/well Blocking Buffer (10% normal goat sera/PBS/0.1% Tween™), incubated 2 hrs at room temperature and then washed twice. As a first step antibody, 50 μl of either patient samples, positive controls or negative control samples diluted in Blocking Buffer were added. Positive controls included WTC19 and WT180 antibodies (Santa Cruz). Negative controls included sera from healthy volunteers. Test samples included sera derived from malignant mesothelioma patients. Plates were incubated overnight at 4° C. or 4 hrs at room temperature, and then washed four times using plate washer. As a second step antibody, anti rabbit HRP (1:5000) (for the positive control) or anti-human HRP (1:8000) Ab at 100 μl/well diluted in Blocking Buffer were added. Plates were incubated for 2 hrs at room temperature and then washed six times.

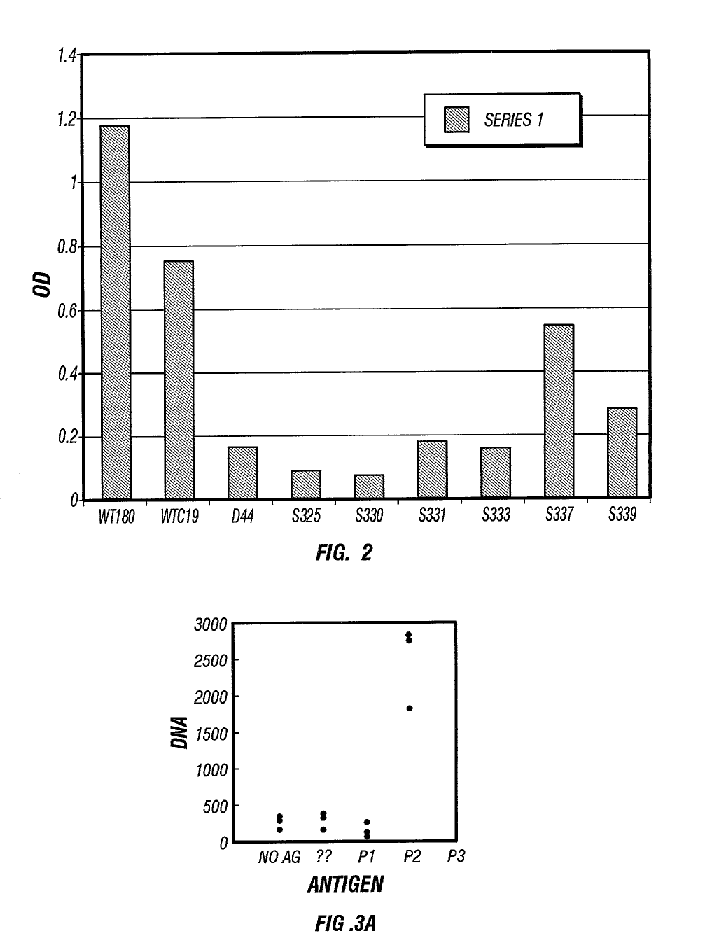

The results are presented in FIG. 2, which shows the detection of WT1-specific anti-bodies in mesothelioma patients. The first two columns show the positive controls (WTC19 and WT180). The third column (designated D44) shows the results for normal control serum. The remaining columns show the results for serum samples obtained from patients with malignant mesothelioma. Serum samples designated S337 and S339 had values that were greater than twice the mean of the normal control samples, and thus were considered positive for malignant mesothelioma.

5.2 Example 2

### Induction of Antibodies to WT1 in Mice Immunized with Cell Lines Expressing WT1

This example illustrates the use of cells expressing WT1 to induce a WT1 specific antibody response in vivo.

Detection of existent antibodies to WT1 in patients with leukemia strongly implied that it is possible to immunize to WT1 protein to elicit immunity to WT1. To test whether immunity to WT1 can be generated by vaccination, mice were injected with TRAMP-C, a WT1 positive tumor cell line of B6 origin. Briefly, male B6 mice were immunized with 5×10⁶ TRAMP-C cells subcutaneously and boosted twice with 5×10⁶ cells at three week intervals. Three weeks after the final immunization, sera were obtained and single cell suspensions of spleens were prepared in RPMI 1640 medium (GIBCO) with 25 μM β-2-mercaptoethanol, 200 units of penicillin per ml, 10 mM L-glutamine, and 10% fetal bovine serum.

Following immunization to TRAMP-C, a WT1 specific antibody response in the immunized animals was detectable. A Western blot was performed illustrating the detection of a WT1-specific antibody response in B6 mice immunized with TRAMP-C, a WT1 positive tumor cell line. Lanes 1, 3 and 5 contained molecular weight markers, and lanes 2, 4 and 6 contained a WT1-specific positive control (NI80, Santa Cruz Biotechnology, polypeptide spanning 180 amino acids of the N-terminal region of the WT1 protein, migrating on the Western blot at approximately 52 kDa). The primary antibody used was WT1 80 in lane 2, sera of non-immunized B6 mice in lane 4 and sera of the immunized B6 mice in lane 6. These results demonstrated that immunization to WT1 polypeptide elicits an immune response to the WT1 polypeptide.

### 5.3 Example 3

### Induction of TH and Antibody Responses in Mice Immunized with WT1 Peptides

This example illustrates the ability of immunization with WT1 peptides to elicit an immune response specific for WT1.

Peptides suitable for eliciting Ab and proliferative T cell responses were identified according to the Tsites program (Rothbard and Taylor, 1988; Deavin et al., 1996), which searches for peptide motifs that have the potential to elicit Th responses. Peptides shown in Table 2 were synthesized and sequenced.

TABLE 2

EXEMPLARY WT1 PEPTIDES

| | | |
|---|---|---|
| Peptide | Sequence | Comments |

| | | |
|---|---|---|
| Mouse: p6-22 | RDLNALLPAVSSLGGGG | 1 mismatch relative to |
| | (SEQ ID NO:13) | human WT1 sequence |
| Human: p6-22 | RDLNALLPAVPSLGGGG | |
| | (SEQ ID NO:1) | |
| Human/mouse: p117-139 | PSQASSGQARMFPNAPYLPSCLE | |
| | (SEQ ID NO:2 and SEQ ID NO:3) | |
| Mouse: p244-262 | GATLKGMAAGSSSSVKWTE | 1 mismatch relative to |
| | (SEQ ID NO:14) | human WT1 sequence |
| Human: p244-262 | GATLKGVAAGSSSSVKWTE | |
| | (SEQ ID NO:4) | |
| Human/mouse: p287-301 | RIHTHGVFRGIQDVR | |
| | (SEQ ID NO:15 and SEQ ID NO:16) | |
| Mouse: p299-313 | VRRVSGVAPTLVRS | 1 mismatch relative to |
| | (SEQ ID NO: 17) | human WT1 sequence |
| Human/mouse: p421-435 | CQKKFARSDELVRHH | |
| | (SEQ ID NO:19 and SEQ ID NO:20) | |

For immunization, peptides were grouped as follows:
Group A: p6-22 human: 10.9 mg in 1 ml (10 μl = 100 μg)
p1 17-139 human/mouse: 7.6 mg in 1 ml (14 μl=100 μg)
p244-262 human: 4.6 mg in 1 ml (22 μl = 100 μg)
Group B: p287-301 human/mouse: 7.2 mg in 1 ml (14 μl = 100 μg)
mouse p299-313: 6.6 mg in 1 ml (15 μl = 100 μg)
p421-435 human/mouse: 3.3 mg in 1 ml (30 μl=100 μg)
Control: (FBL peptide 100 μg) + CFA/IFA
Control: (CD45 peptide 100 μg) + CFA/IFA

Group A contained peptides present within the amino terminus portion of WT1 (exon 1) and Group B contained peptides present within the carboxy terminus, which contains a four zinc finger region with sequence homology to other DNA-binding proteins. Within group B, p287-301 and p299-313 were derived from exon 7, zinc finger 1, and p421-435 was derived from exon 10, zinc finger IV.

B6 mice were immunized with a group of WT1 peptides or with a control peptide. Peptides were dissolved in 1 ml sterile water for injection, and B6 mice were immunized 3 times at time intervals of three weeks. Adjuvants used were CFA/IFA, GM-CSF, and Montinide. The presence of antibodies specific for WT1 was then determined as described in Examples 1 and 2, and proliferative T cell responses were evaluated using a standard thymidine incorporation assay, in which cells were cultured in the presence of antigen and proliferation was evaluated by measuring incorporated radioactivity (Chen et al., 1994). In particular, lymphocytes were cultured in 96-well plates at 2×10⁵ cells per well with 4×10⁵ irradiated (3000 rads) syngeneic spleen cells and the designated peptide.

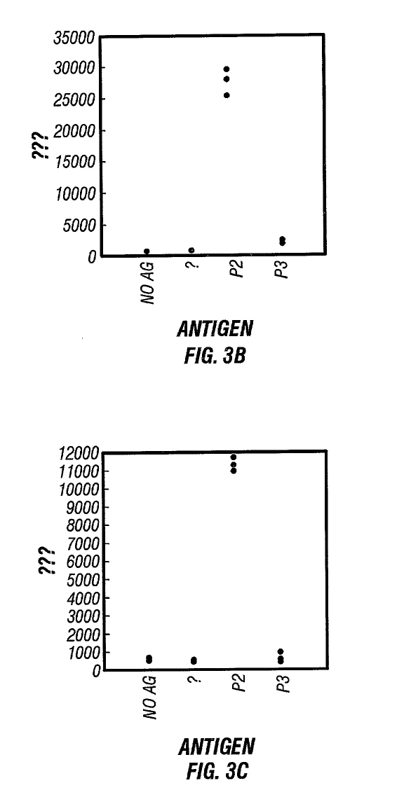

Immunization of mice with the group of peptides designated as Group A elicited an antibody response to WT1. A representative Western blot was performed that illustrated the detection of WT1-specific antibodies in mice immunized with representative WT1 peptides. Lanes 1, 3 and 5 contained molecular weight markers, with lanes 2, 4 and 6 showing a WT1 specific positive control (N180, Santa Cruz Biotechnology, polypeptide spanning 180 amino acids of the N-terminal region of the WT1 protein, migrating on the Western blot at 52 kDa). The primary antibody used was WT180 in lane 2, sera of non-immunized B6 mice in lane 4 and sera of the immunized B6 mice in lane 6. No antibodies were detected following immunization to Vaccine B, which is consistent with a lack of helper T cell response from immunization with Vaccine B. P117-139 elicited proliferative T cell responses (FIG. 3A, FIG. 3B, and FIG. 3C). The stimulation indices (SI) varied between 8 and 72. Other peptides (P6-22 and P299-313) also were shown to elicit proliferative T cell responses. Immunization with P6-22 resulted in a stimulation index (SI) of 2.3 and immunization with P299-313 resulted in a SI of 3.3. Positive controls included ConA stimulated T cells, as well as T cells stimulated with known antigens, such as CD45 and FBL, and allogeneic T cell lines (DeBruijn et al., 1991).

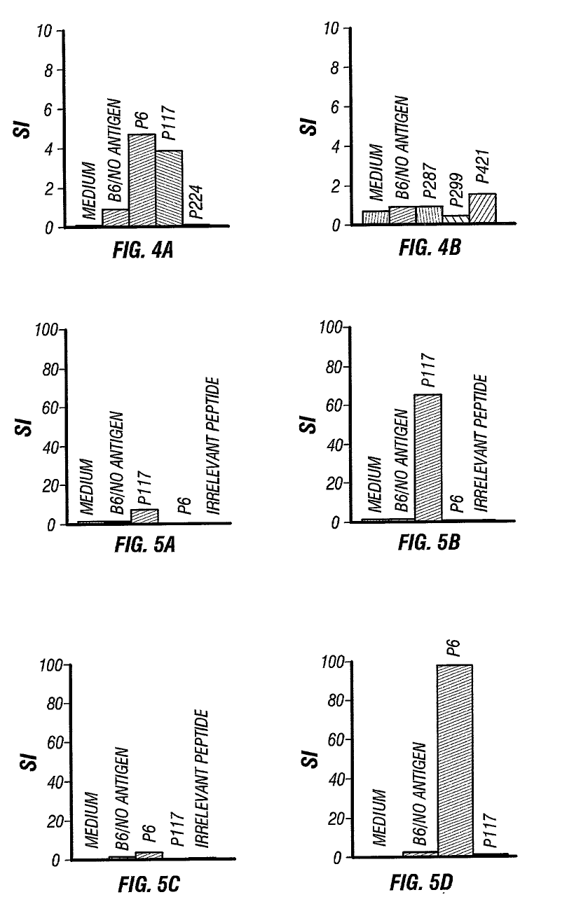

FIG. 4A and FIG. 4B show the proliferative response observed for each of the three peptides within vaccine A (FIG. 4A) and vaccine B (FIG. 4B). Vaccine A elicited proliferative T cell responses to the immunizing peptides p6-22 and pl 17-139, with stimulation indices (SI) varying between 3 and 8 (bulk lines). No proliferative response to p244-262 was detected (FIG. 4A).

Subsequent in vitro stimulations were carried out as single peptide stimulations using only p6-22 and pl 17-139. Stimulation of the Vaccine A specific T cell line with pl 17-139 resulted in proliferation to pl 17-139 with no response to p6-22 (FIG. 5A). Clones derived from the line were specific for p117-139 (FIG. 5B). By contrast, stimulation of the Vaccine A specific T cell line with p6-22 resulted in proliferation to p6-22 with no response to p117-139 (FIG. 5C). Clones derived from the line were specific for p6-22 (FIG. 5D). These results demonstrated that vaccination with WT1 peptides can elicit an antibody response to WT1 polypeptides, and a proliferative T cell response to the immunizing peptides.

### 5.4 Example 4

### Induction of CTL Responses in Mice Immunized with WT1 Peptides

This example illustrates the ability of WT1 peptides to elicit CTL immunity.

Nonameric peptides (9-mers) with motifs appropriate for binding to class I MHC were identified using a variety of analytical methods, including BIMAS HLA peptide binding prediction analysis (Parker et al., 1994). Peptides identified within such analyses are shown in Table 3 -Table 45. In each of these tables, the score reflects the theoretical binding affinity (half-time of dissociation) of the peptide to the MHC molecule indicated. Peptides identified using the TSITES program (Rothbard and Taylor, 1988; Deavin et al., 1996), which searches for peptide motifs that have the potential to elicit Th responses are further shown in Table 46.

TABLE 3

RESULTS OF BIMAS HLA PEPTIDE BINDING PREDICTION ANALYSIS FOR BINDING OF
HUMAN WT1 PEPTIDES TO HUMAN HLA A1

| | | | |
|---|---|---|---|
| | | | Score (Estimate of Half Time of |
| | Start | | Disassociation of a Molecule |
| Rank | Position | Subsequence | Containing This Subsequence) |

| | | | | |
|---|---|---|---|---|
| 1 | 137 | CLESQPAIR | (SEQ ID NO:47) | 18.000 |
| 2 | 80 | GAEPHEEQC | (SEQ ID NO:87) | 9.000 |
| 3 | 40 | FAPPGASAY | (SEQ ID NO:74) | 5.000 |
| 4 | 354 | QCDFKDCER | (SEQ ID NO:162) | 5.000 |
| 5 | 2 | GSDVRDLNA | (SEQ ID NO:101) | 3.750 |
| 6 | 152 | VTFDGTPSY | (SEQ ID NO:244) | 2.500 |
| 7 | 260 | WTEGQSNHS | (SEQ ID NO:247) | 2.250 |
| 8 | 409 | TSEKPFSCR | (SEQ ID NO:232) | 1.350 |
| 9 | 73 | KQEPSWGGA | (SEQ ID NO:125) | 1.350 |
| 10 | 386 | KTCQRKFSR | (SEQ ID NO:128) | 1.250 |
| 11 | 37 | VLDFAPPGA | (SEQ ID NO:241) | 1.000 |
| 12 | 325 | CAYPGCNKR | (SEQ ID NO:44) | 1.000 |
| 13 | 232 | QLECMTWNQ | (SEQ ID NO:167) | 0.900 |
| 14 | 272 | ESDNHTTPI | (SEQ ID NO:71) | 0.750 |
| 15 | 366 | RSDQLKRHQ | (SEQ ID NO:193) | 0.750 |
| 16 | 222 | SSDNLYQMT | (SEQ ID NO:217) | 0.750 |
| 17 | 427 | RSDELVRHH | (SEQ ID NO:191) | 0.750 |
| 18 | 394 | RSDHLKTHT | (SEQ ID NO:192) | 0.750 |
| 19 | 317 | TSEKRPFMC | (SEQ ID NO:233) | 0.675 |
| 20 | 213 | QALLLRTPY | (SEQ ID NO:160) | 0.500 |

TABLE 4

RESULTS OF BIMAS HLA PEPTIDE BINDING PREDICTION ANALYSIS FOR BINDING OF
HUMAN WT1 PEPTIDES TO HUMAN HLA A 0201

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 126 | RMFPNAPYL | (SEQ ID NO:185) | 313.968 |
| 2 | 187 | SLGEQQYSV | (SEQ ID NO:214) | 285.163 |
| 3 | 10 | ALLPAVPSL | (SEQ ID NO:34) | 181.794 |
| 4 | 242 | NLGATLKGV | (SEQ ID NO:146) | 159.970 |
| 5 | 225 | NLYQMTSQL | (SEQ ID NO:147) | 68.360 |
| 6 | 292 | GVFRGIQDV | (SEQ ID NO:103) | 51.790 |
| 7 | 191 | QQYSVPPPV | (SEQ ID NO:171) | 22.566 |
| 8 | 280 | ILCGAQYRI | (SEQ ID NO:116) | 17.736 |
| 9 | 235 | CMTWNQMNL | (SEQ ID NO:49) | 15.428 |
| 10 | 441 | NMTKLQLAL | (SEQ ID NO:149) | 15.428 |
| 11 | 7 | DLNALLPAV | (SEQ ID NO:58) | 11.998 |
| 12 | 227 | YQMTSQLEC | (SEQ ID NO:251) | 8.573 |
| 13 | 239 | NQMNLGATL | (SEQ ID NO:151) | 8.014 |
| 14 | 309 | TLVRSASET | (SEQ ID NO:226) | 7.452 |
| 15 | 408 | KTSEKPFSC | (SEQ ID NO:129) | 5.743 |
| 16 | 340 | LQMHSRKHT | (SEQ ID NO:139) | 4.752 |
| 17 | 228 | QMTSQLECM | (SEQ ID NO:169) | 4.044 |
| 18 | 93 | TVHFSGQFT | (SEQ ID NO:235) | 3.586 |
| 19 | 37 | VLDFAPPGA | (SEQ ID NO:241) | 3.378 |
| 20 | 86 | EQCLSAFTV | (SEQ ID NO:69) | 3.068 |

TABLE 5

RESULTS OF BIMAS HLA PEPTIDE BINDING PREDICTION ANALYSIS FOR BINDING OF
HUMAN WT1 PEPTIDES TO HUMAN HLA A 0205

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 10 | ALLPAVPSL | (SEQ ID NO:34) | 42.000 |
| 2 | 292 | GVFRGIQDV | (SEQ ID NO:103) | 24.000 |
| 3 | 126 | RMFPNAPYL | (SEQ ID NO:185) | 21.000 |
| 4 | 225 | NLYQMTSQL | (SEQ ID NO:147) | 21.000 |
| 5 | 239 | NQMNLGATL | (SEQ ID NO:151) | 16.800 |
| 6 | 302 | RVPGVAPTL | (SEQ ID NO:195) | 14.000 |
| 7 | 441 | NMTKLQLAL | (SEQ ID NO:149) | 7.000 |
| 8 | 235 | CMTWNQMNL | (SEQ ID NO:49) | 7.000 |
| 9 | 187 | SLGEQQYSV | (SEQ ID NO:214) | 6.000 |
| 10 | 191 | QQYSVPPPV | (SEQ ID NO:171) | 4.800 |
| 11 | 340 | LQMHSRKHT | (SEQ ID NO:139) | 4.080 |
| 12 | 242 | NLGATLKGV | (SEQ ID NO:146) | 4.000 |
| 13 | 227 | YQMTSQLEC | (SEQ ID NO:251) | 3.600 |
| 14 | 194 | SVPPPVYGC | (SEQ ID NO:218) | 2.000 |
| 15 | 93 | TVHFSGQFT | (SEQ ID NO:235) | 2.000 |
| 16 | 280 | ILCGAQYRI | (SEQ ID NO:116) | 1.700 |
| 17 | 98 | GQFTGTAGA | (SEQ ID NO:99) | 1.200 |
| 18 | 309 | TLVRSASET | (SEQ ID NO:226) | 1.000 |
| 19 | 81 | AEPHEEQCL | (SEQ ID NO:30) | 0.980 |
| 20 | 73 | KQEPSWGGA | (SEQ ID NO:125) | 0.960 |

TABLE 6

RESULTS OF BIMAS HLA PEPTIDE BINDING PREDICTION ANALYSIS FOR BINDING OF
HUMAN WT1 PEPTIDES TO HUMAN HLA A24

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 302 | RVPGVAPTL | (SEQ ID NO:195) | 16.800 |
| 2 | 218 | RTPYSSDNL | (SEQ ID NO:194) | 12.000 |
| 3 | 356 | DFKDCERRF | (SEQ ID NO:55) | 12.000 |
| 4 | 126 | RMFPNAPYL | (SEQ ID NO:185) | 9.600 |
| 5 | 326 | AYPGCNKRY | (SEQ ID NO:42) | 7.500 |
| 6 | 270 | GYESDNHT | (SEQ ID NO:106) | 7.500 |
| 7 | 239 | NQMNLGATL | (SEQ ID NO:151) | 7.200 |
| 8 | 10 | ALLPAVPSL | (SEQ ID NO:34) | 7.200 |
| 9 | 130 | NAPYLPSCL | (SEQ ID NO:144) | 7.200 |
| 10 | 329 | GCNKRYFKL | (SEQ ID NO:90) | 6.600 |
| 11 | 417 | RWPSCQKKF | (SEQ ID NO:196) | 6.600 |
| 12 | 47 | AYGSLGGPA | (SEQ ID NO:41) | 6.000 |
| 13 | 180 | DPMGQQGSL | (SEQ ID NO:59) | 6.000 |
| 14 | 104 | DVRDLNALL | (SEQ ID NO:62) | 5.760 |
| 15 | 285 | QYRIHTHGV | (SEQ ID NO:175) | 5.000 |
| 16 | 192 | QYSVPPPVY | (SEQ ID NO:176) | 5.000 |
| 17 | 207 | DSCTGSQAL | (SEQ ID NO:61) | 4.800 |
| 18 | 441 | NMTKLQLAL | (SEQ ID NO:149) | 4.800 |
| 19 | 225 | NLYQMTSQL | (SEQ ID NO:147) | 4.000 |
| 20 | 235 | CMTWNQMNL | (SEQ ID NO:49) | 4.000 |

TABLE 7

RESULTS OF BIMAS HLA PEPTIDE BINDING PREDICTION ANALYSIS FOR BINDING OF
HUMAN WT1 PEPTIDES TO HUMAN HLA A3

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 436 | NMHQRNMTK | (SEQ ID NO:148) | 40.000 |
| 2 | 240 | QMNLGATLK | (SEQ ID NO:168) | 20.000 |
| 3 | 88 | CLSAFTVHF | (SEQ ID NO:48) | 6.000 |
| 4 | 126 | RMFPNAPYL | (SEQ ID NO:185) | 4.500 |
| 5 | 169 | AQFPNHSFK | (SEQ ID NO:36) | 4.500 |
| 6 | 10 | ALLPAVPSL | (SEQ ID NO:34) | 4.050 |
| 7 | 137 | CLESQPAIR | (SEQ ID NO:47) | 4.000 |
| 8 | 225 | NLYQMTSQL | (SEQ ID NO:147) | 3.000 |
| 9 | 32 | AQWAPVLDF | (SEQ ID NO:37) | 2.700 |
| 10 | 280 | ILCGAQYRI | (SEQ ID NO:116) | 2.700 |
| 11 | 386 | KTCQRKFSR | (SEQ ID NO:128) | 1.800 |
| 12 | 235 | CMTWNQMNL | (SEQ ID NO:49) | 1.200 |
| 13 | 441 | NMTKLQLAL | (SEQ ID NO:149) | 1.200 |
| 14 | 152 | VTFDGTPSY | (SEQ ID NO:244) | 1.000 |
| 15 | 187 | SLGEQQYSV | (SEQ ID NO:214) | 0.900 |
| 16 | 383 | FQCKTCQRK | (SEQ ID NO:80) | 0.600 |
| 17 | 292 | GVFRGIQDV | (SEQ ID NO:103) | 0.450 |
| 18 | 194 | SVPPPHYGC | (SEQ ID NO:218) | 0.405 |
| 19 | 287 | RIHTHGVFR | (SEQ ID NO:182) | 0.400 |
| 20 | 263 | GQSNHSTGY | (SEQ ID NO:100) | 0.360 |

TABLE 8

RESULTS OF BIMAS HLA PEPTIDE BINDING PREDICTION ANALYSIS FOR BINDING OF
HUMAN WT1 PEPTIDES TO HUMAN HLA A68.1

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 100 | FTGTAGACR | (SEQ ID NO:84) | 100.000 |
| 2 | 386 | KTCQRKFSR | (SEQ ID NO:128) | 50.000 |
| 3 | 368 | DQLKRHQRR | (SEQ ID NO:60) | 30.000 |
| 4 | 312 | RSASETSEK | (SEQ ID NO:190) | 18.000 |
| 5 | 337 | LSHLQMHSR | (SEQ ID NO:141) | 15.000 |
| 6 | 364 | FSRSDQLKR | (SEQ ID NO:83) | 15.000 |
| 7 | 409 | TSEKPFSCR | (SEQ ID NO:232) | 15.000 |
| 8 | 299 | DVRRVPGVA | (SEQ ID NO:63) | 12.000 |
| 9 | 4 | DVRDLNALL | (SEQ ID NO:62) | 12.000 |
| 10 | 118 | SQASSGQAR | (SEQ ID NO:216) | 10.000 |
| 11 | 343 | HSRKHTGEK | (SEQ ID NO:111) | 9.000 |
| 12 | 169 | AQFPNHSFK | (SEQ ID NO:36) | 9.000 |
| 13 | 292 | GVFRGIQDV | (SEQ ID NO:103) | 8.000 |
| 14 | 325 | CAYPGCNKR | (SEQ ID NO:44) | 7.500 |
| 15 | 425 | FARSDELVR | (SEQ ID NO:75) | 7.500 |
| 16 | 354 | QCDFKDCER | (SEQ ID NO:162) | 7.500 |
| 17 | 324 | MCAYPGCNK | (SEQ ID NO:142) | 6.000 |
| 18 | 251 | AAGSSSSVK | (SEQ ID NO:28) | 6.000 |
| 19 | 379 | GVKPFQCKT | (SEQ ID NO:104) | 6.000 |
| 20 | 137 | CLESQPAIR | (SEQ ID NO:47) | 5.000 |

TABLE 9

RESULTS OF BIMAS HLA PEPTIDE BINDING PREDICTION ANALYSIS FOR BINDING OF
HUMAN WT1 PEPTIDES TO HUMAN HLA A1

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 386 | KTCQRKFSR | (SEQ ID NO:128) | 1.800 |
| 2 | 169 | AQFPNHSFK | (SEQ ID NO:36) | 1.200 |
| 3 | 436 | NMHQRNMTK | (SEQ ID NO:148) | 0.800 |
| 4 | 391 | KFSRSDHLK | (SEQ ID NO:120) | 0.600 |
| 5 | 373 | HQRRHTGVK | (SEQ ID NO:109) | 0.600 |
| 6 | 383 | FQCKTCQRK | (SEQ ID NO:80) | 0.600 |
| 7 | 363 | RFSRSDQLK | (SEQ ID NO:178) | 0.600 |
| 8 | 240 | QMNLGATLK | (SEQ ID NO:168) | 0.400 |
| 9 | 287 | RIHTHGVFR | (SEQ ID NO:182) | 0.240 |
| 10 | 100 | FTGTAGACR | (SEQ ID NO:84) | 0.200 |
| 11 | 324 | MCAYPGCNK | (SEQ ID NO:142) | 0.200 |
| 12 | 251 | AAGSSSSVK | (SEQ ID NO:28) | 0.200 |
| 13 | 415 | SCRWPSCQK | (SEQ ID NO:201) | 0.200 |
| 14 | 118 | SQASSGQAR | (SEQ ID NO:216) | 0.120 |
| 15 | 292 | GVFRGIQDV | (SEQ ID NO:103) | 0.120 |
| 16 | 137 | CLESQPAIR | (SEQ ID NO:47) | 0.080 |
| 17 | 425 | FARSDELVR | (SEQ ID NO:75) | 0.080 |
| 18 | 325 | CAYPGCNKR | (SEQ ID NO:44) | 0.080 |
| 19 | 312 | RSASETSEK | (SEQ ID NO:190) | 0.060 |
| 20 | 65 | PPPPHSFIK | (SEQ ID NO:156) | 0.060 |

TABLE 10

RESULTS OF BIMAS HLA PEPTIDE BINDING PREDICTION ANALYSIS FOR BINDING OF
HUMAN WT1 PEPTIDES TO HUMAN ULA A 3101

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 386 | KTCQRKFSR | (SEQ ID NO:128) | 9.000 |
| 2 | 287 | RIHTHGVFR | (SEQ ID NO:182) | 6.000 |
| 3 | 137 | CLESQPAIR | (SEQ ID NO:47) | 2.000 |
| 4 | 118 | SQASSGQAR | (SEQ ID NO:216) | 2.000 |
| 5 | 368 | DQLKRHQRR | (SEQ ID NO:60) | 1.200 |
| 6 | 100 | FTGTAGACR | (SEQ ID NO:84) | 1.000 |
| 7 | 293 | VFRGIQDVR | (SEQ ID NO:238) | 0.600 |
| 8 | 325 | CAYPGCNKR | (SEQ ID NO:44) | 0.600 |
| 9 | 169 | AQFPNHSFK | (SEQ ID NO:36) | 0.600 |
| 10 | 279 | PILCGAQYR | (SEQ ID NO:155) | 0.400 |
| 11 | 436 | NMHQRNMTK | (SEQ ID NO:148) | 0.400 |
| 12 | 425 | FARSDELVR | (SEQ ID NO:75) | 0.400 |
| 13 | 32 | AQWAPVLDF | (SEQ ID NO:37) | 0.240 |
| 14 | 240 | QMNLGATLK | (SEQ ID NO:168) | 0.200 |
| 15 | 354 | QCDFKDCER | (SEQ ID NO:162) | 0.200 |
| 16 | 373 | HQRRHTGVK | (SEQ ID NO:109) | 0.200 |
| 17 | 383 | FQCKTCQRK | (SEQ ID NO:80) | 0.200 |
| 18 | 313 | SASETSEKR | (SEQ ID NO:197) | 0.200 |
| 19 | 358 | KDCERRFSR | (SEQ ID NO:118) | 0.180 |
| 20 | 391 | KFSRSDHLK | (SEQ ID NO:120) | 0.180 |

TABLE 11

RESULTS OF BIMAS HLA PEPTIDE BINDING PREDICTION ANALYSIS FOR BINDING OF
HUMAN WT1 PEPTIDES TO HUMAN HLA A 3302

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 337 | LSHLQMHSR | (SEQ ID NO:141) | 15.000 |
| 2 | 409 | TSEKPFSCR | (SEQ ID NO:232) | 15.000 |
| 3 | 364 | FSRSDQLKR | (SEQ ID NO:83) | 15.000 |
| 4 | 137 | CLESQPAIR | (SEQ ID NO:47) | 9.000 |
| 5 | 368 | DQLKRHQRR | (SEQ ID NO:60) | 9.000 |
| 6 | 287 | RIHTHGVFR | (SEQ ID NO:182) | 4.500 |
| 7 | 210 | TGSQALLLR | (SEQ ID NO:223) | 3.000 |
| 8 | 425 | FARSDELVR | (SEQ ID NO:75) | 3.000 |
| 9 | 313 | SASETSEKR | (SEQ ID NO:197) | 3.000 |
| 10 | 293 | VFRGIQDVR | (SEQ ID NO:238) | 3.000 |
| 11 | 354 | QCDFKDCER | (SEQ ID NO:162) | 3.000 |
| 12 | 100 | FTGTAGACR | (SEQ ID NO:84) | 3.000 |
| 13 | 118 | SQASSGQAR | (SEQ ID NO:216) | 3.000 |
| 14 | 325 | CAYPGCNKR | (SEQ ID NO:44) | 3.000 |
| 15 | 207 | DSCTGSQAL | (SEQ ID NO:61) | 1.500 |
| 16 | 139 | ESQPAIRNQ | (SEQ ID NO:72) | 1.500 |
| 17 | 299 | DVRRVPGVA | (SEQ ID NO:63) | 1.500 |
| 18 | 419 | PSCQKKFAR | (SEQ ID NO:159) | 1.500 |
| 19 | 272 | ESDNHTTPI | (SEQ ID NO:71) | 1.500 |
| 20 | 4 | DVRDLNALL | (SEQ ID NO:62) | 1.500 |

TABLE 12

RESULTS OF BIMAS HLA PEPTIDE BINDING PREDICTION ANALYSIS FOR BINDING OF
HUMAN WT1 PEPTIDES TO HUMAN HLA B14

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 362 | RRFSRSDQL | (SEQ ID NO:187) | 1000.000 |
| 2 | 332 | KRYFKLSHL | (SEQ ID NO:127) | 300.000 |
| 3 | 423 | KKFARSDEL | (SEQ ID NO:122) | 150.000 |
| 4 | 390 | RKFSRSDHL | (SEQ ID NO:183) | 150.000 |
| 5 | 439 | QRNMTKLQL | (SEQ ID NO:173) | 20.000 |
| 6 | 329 | GCNKRYFKL | (SEQ ID NO:90) | 10.000 |
| 7 | 10 | ALLPAVPSL | (SEQ ID NO:34) | 10.000 |
| 8 | 180 | DPMGQQGSL | (SEQ ID NO:59) | 9.000 |
| 9 | 301 | RRVPGVAPT | (SEQ ID NO:189) | 6.000 |
| 10 | 126 | RMFPNAPYL | (SEQ ID NO:185) | 5.000 |
| 11 | 371 | KRHQRRHTG | (SEQ ID NO:126) | 5.000 |
| 12 | 225 | NLYQMTSQL | (SEQ ID NO:147) | 5.000 |
| 13 | 144 | IRNQGYSTV | (SEQ ID NO:117) | 4.000 |
| 14 | 429 | DELVRHHNM | (SEQ ID NO:53) | 3.000 |
| 15 | 437 | MHQRNMTKL | (SEQ ID NO:143) | 3.000 |
| 16 | 125 | ARMFPNAPY | (SEQ ID NO:38) | 3.000 |
| 17 | 239 | NQMNLGATL | (SEQ ID NO:151) | 3.000 |
| 18 | 286 | YRIHTHGVF | (SEQ ID NO:252) | 3.000 |
| 19 | 174 | HSFKHEDPM | (SEQ ID NO:110) | 3.000 |
| 20 | 372 | RHQRRHTGV | (SEQ ID NO:181) | 3.000 |

TABLE 13

RESULTS OF BIMAS HLA PEPTIDE BINDING PREDICTION ANALYSIS FOR BINDING OF
HUMAN WT1 PEPTIDES TO HUMAN HLA B40

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 81 | AEPHEEQCL | (SEQ ID NO:30) | 40.000 |
| 2 | 429 | DELVRHHNM | (SEQ ID NO:53) | 24.000 |
| 3 | 410 | SEKPFSCRW | (SEQ ID NO:207) | 20.000 |
| 4 | 318 | SEKRPFMCA | (SEQ ID NO:208) | 15.000 |
| 5 | 233 | LECMTWNQM | (SEQ ID NO:131) | 12.000 |
| 6 | 3 | SDVRDLNAL | (SEQ ID NO:206) | 10.000 |
| 7 | 349 | GEKPYQCDF | (SEQ ID NO:91) | 8.000 |
| 8 | 6 | RDLNALLPA | (SEQ ID NO:177) | 5.000 |
| 9 | 85 | EEQCLSAFT | (SEQ ID NO:65) | 4.000 |
| 10 | 315 | SETSEKRPF | (SEQ ID NO:209) | 4.000 |
| 11 | 261 | TEGQSNHST | (SEQ ID NO:221) | 4.000 |
| 12 | 23 | GCALPVSGA | (SEQ ID NO:89) | 3.000 |
| 13 | 38 | LDFAPPGAS | (SEQ ID NO:130) | 3.000 |
| 14 | 273 | SDNHTTPIL | (SEQ ID NO:204) | 2.500 |
| 15 | 206 | TDSCTGSQA | (SEQ ID NO:220) | 2.500 |
| 16 | 24 | CALPVSGAA | (SEQ ID NO:43) | 2.000 |
| 17 | 98 | GQFTGTAGA | (SEQ ID NO:99) | 2.000 |
| 18 | 30 | GAAQWAPVL | (SEQ ID NO:86) | 2.000 |
| 19 | 84 | HEEQCLSAF | (SEQ ID NO:107) | 2.000 |
| 20 | 26 | LPVSGAAQW | (SEQ ID NO:138) | 2.000 |

TABLE 14

RESULTS OF BIMAS HLA PEPTIDE BINDING PREDICTION ANALYSIS FOR BINDING OF
HUMAN WT1 PEPTIDES TO HUMAN HLA B60

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 81 | AEPHEEQCL | (SEQ ID NO:30) | 160.000 |
| 2 | 3 | SDVRDLNAL | (SEQ ID NO:206) | 40.000 |
| 3 | 429 | DELVRHHNM | (SEQ ID NO:53) | 40.000 |
| 4 | 233 | LECMTWNQM | (SEQ ID NO:131) | 22.000 |
| 5 | 273 | SDNHTTPIL | (SEQ ID NO:204) | 20.000 |
| 6 | 209 | CTGSQALLL | (SEQ ID NO:52) | 8.000 |
| 7 | 30 | GAAQWAPVL | (SEQ ID NO:86) | 8.000 |
| 8 | 318 | SEKRPFMCA | (SEQ ID NO:208) | 8.000 |
| 9 | 180 | DPMGQQGSL | (SEQ ID NO:59) | 8.000 |
| 10 | 138 | LESQPAIRN | (SEQ ID NO:132) | 5.280 |
| 11 | 239 | NQMNLGATL | (SEQ ID NO:151) | 4.400 |
| 12 | 329 | GCNKRYFKL | (SEQ ID NO:90) | 4.400 |
| 13 | 130 | NAPYLPSCL | (SEQ ID NO:144) | 4.400 |
| 14 | 85 | EEQCLSAFT | (SEQ ID NO:65) | 4.400 |
| 15 | 208 | SCTGSQALL | (SEQ ID NO:202) | 4.000 |
| 16 | 207 | DSCTGSQAL | (SEQ ID NO:61) | 4.000 |
| 17 | 218 | RTPYSSDNL | (SEQ ID NO:194) | 4.000 |
| 18 | 261 | TEGQSNHST | (SEQ ID NO:221) | 4.000 |
| 19 | 18 | LGGGGGCAL | (SEQ ID NO:134) | 4.000 |
| 20 | 221 | YSSDNLYQM | (SEQ ID NO:253) | 2.200 |

TABLE 15

RESULTS OF BIMAS HLA PEPTIDE BINDING PREDICTION ANALYSIS FOR BINDING OF
HUMAN WT1 PEPTIDES TO HUMAN HLA B61

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 318 | SEKRPFMCA | (SEQ ID NO:208) | 20.000 |
| 2 | 429 | DELVRHHNM | (SEQ ID NO:53) | 16.000 |
| 3 | 298 | QDVRRVPGV | (SEQ ID NO:164) | 10.000 |
| 4 | 81 | AEPHEEQCL | (SEQ ID NO:30) | 8.000 |
| 5 | 233 | LECMTWNQM | (SEQ ID NO:131) | 8.000 |
| 6 | 6 | RDLNALLPA | (SEQ ID NO:177) | 5.500 |
| 7 | 85 | EEQCLSAFT | (SEQ ID NO:65) | 4.000 |
| 8 | 261 | TEGQSNHST | (SEQ ID NO:221) | 4.000 |
| 9 | 206 | TDSCTGSQA | (SEQ ID NO:220) | 2.500 |
| 10 | 295 | RGIQDVRRV | (SEQ ID NO:179) | 2.200 |
| 11 | 3 | SDVRDLNAL | (SEQ ID NO:206) | 2.000 |
| 12 | 250 | VAAGSSSSV | (SEQ ID NO:236) | 2.000 |
| 13 | 29 | SGAAQWAPV | (SEQ ID NO:211) | 2.000 |
| 14 | 315 | SETSEKRPF | (SEQ ID NO:209) | 1.600 |
| 15 | 138 | LESQPAIRN | (SEQ ID NO:132) | 1.200 |
| 16 | 244 | GATLKGVAA | (SEQ ID NO:88) | 1.100 |
| 17 | 20 | GGGGCALPV | (SEQ ID NO:92) | 1.100 |
| 18 | 440 | RNMTKLQLA | (SEQ ID NO:186) | 1.100 |
| 19 | 23 | GCALPVSGA | (SEQ ID NO:89) | 1.100 |
| 20 | 191 | QQYSVPPPV | (SEQ ID NO:171) | 1.000 |

TABLE 16

RESULTS OF BIMAS HLA PEPTIDE BINDING PREDICTION ANALYSIS FOR BINDING OF
HUMAN WT1 PEPTIDES TO HUMAN HLA B62

| | | | |
|---|---|---|---|
| | hc,4 | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 146 | NQGYSTVTF | (SEQ ID NO:150) | 211.200 |
| 2 | 32 | AQWAPVLDF | (SEQ ID NO:37) | 96.000 |
| 3 | 263 | GQSNHSTGY | (SEQ ID NO:100) | 96.000 |
| 4 | 88 | CLSAFTVHF | (SEQ ID NO:48) | 96.000 |
| 5 | 17 | SLGGGGGCA | (SEQ ID NO:215) | 9.600 |
| 6 | 239 | NQMNLGATL | (SEQ ID NO:151) | 8.800 |
| 7 | 191 | QQYSVPPPV | (SEQ ID NO:171) | 8.000 |
| 8 | 98 | GQFTGTAGA | (SEQ ID NO:99) | 8.000 |
| 9 | 384 | QCKTCQRKF | (SEQ ID NO:163) | 6.000 |
| 10 | 40 | FAPPGASAY | (SEQ ID NO:74) | 4.800 |
| 11 | 227 | YQMTSQLEC | (SEQ ID NO:251) | 4.800 |
| 12 | 187 | SLGEQQYSV | (SEQ ID NO:214) | 4.400 |
| 13 | 86 | EQCLSAFTV | (SEQ ID NO:69) | 4.400 |
| 14 | 152 | VTFDGTPSY | (SEQ ID NO:244) | 4.400 |
| 15 | 101 | TGTAGACRY | (SEQ ID NO:224) | 4.000 |
| 16 | 242 | NLGATLKGV | (SEQ ID NO:146) | 4.000 |
| 17 | 92 | FTVHFSGQF | (SEQ ID NO:85) | 4.000 |
| 18 | 7 | DLNALLPAV | (SEQ ID NO:58) | 4.000 |
| 19 | 123 | GQARMFPNA | (SEQ ID NO:98) | 4.000 |
| 20 | 280 | ILCGAQYRI | (SEQ ID NO:116) | 3.120 |

TABLE 17

RESULTS OF BIMAS HLA PEPTIDE BINDING PREDICTION ANALYSIS FOR BINDING OF
HUMAN WT1 PEPTIDES TO HUMAN HLA B7

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 180 | DPMGQQGSL | (SEQ ID NO:59) | 240.000 |
| 2 | 4 | DVRDLNALL | (SEQ ID NO:62) | 200.000 |
| 3 | 302 | RVPGVAPTL | (SEQ ID NO:195) | 20.000 |
| 4 | 30 | GAAQWAPVL | (SEQ ID NO:86) | 12.000 |
| 5 | 239 | NQMNLGATL | (SEQ ID NO:151) | 12.000 |
| 6 | 130 | NAPYLPSCL | (SEQ ID NO:144) | 12.000 |
| 7 | 10 | ALLPAVPSL | (SEQ ID NO:34) | 12.000 |
| 8 | 299 | DVRRVPGVA | (SEQ ID NO:63) | 5.000 |
| 9 | 208 | SCTGSQALL | (SEQ ID NO:202) | 4.000 |
| 10 | 303 | VPGVAPTLV | (SEQ ID NO:242) | 4.000 |
| 11 | 18 | LGGGGGCAL | (SEQ ID NO:134) | 4.000 |
| 12 | 218 | RTPYSSDNL | (SEQ ID NO:194) | 4.000 |
| 13 | 207 | DSCTGSQAL | (SEQ ID NO:61) | 4.000 |
| 14 | 209 | CTGSQALLL | (SEQ ID NO:52) | 4.000 |
| 15 | 329 | GCNKRYFKL | (SEQ ID NO:90) | 4.000 |
| 16 | 235 | CMTWNQMNL | (SEQ ID NO:49) | 4.000 |
| 17 | 441 | NMTKLQLAL | (SEQ ID NO:149) | 4.000 |
| 18 | 126 | RMFPNAPYL | (SEQ ID NO:185) | 4.000 |
| 19 | 225 | NLYQMTSQL | (SEQ ID NO:147) | 4.000 |
| 20 | 143 | AIRNQGYST | (SEQ ID NO:33) | 3.000 |

TABLE 18

RESULTS OF BIMAS HLA PEPTIDE BINDING PREDICTION ANALYSIS FOR BINDING OF
HUMAN WT1 PEPTIDES TO HUMAN HLA B8

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 329 | GCNKRYFKL | (SEQ ID NO:90) | 16.000 |
| 2 | 4 | DVRDLNALL | (SEQ ID NO:62) | 12.000 |
| 3 | 316 | ETSEKRPFM | (SEQ ID NO:73) | 3.000 |
| 4 | 180 | DPMGQQGSL | (SEQ ID NO:59) | 1.600 |
| 5 | 208 | SCTGSQALL | (SEQ ID NO:202) | 0.800 |
| 6 | 130 | NAPYLPSCL | (SEQ ID NO:144) | 0.800 |
| 7 | 244 | GATLKGVAA | (SEQ ID NO:88) | 0.800 |
| 8 | 30 | GAAQWAPVL | (SEQ ID NO:86) | 0.800 |
| 9 | 299 | DVRRVPGVA | (SEQ ID NO:63) | 0.400 |
| 10 | 420 | SCQKKFARS | (SEQ ID NO:200) | 0.400 |
| 11 | 387 | TCQRKFSRS | (SEQ ID NO:219) | 0.400 |
| 12 | 225 | NLYQMTSQL | (SEQ ID NO:147) | 0.400 |
| 13 | 141 | QPAIRNQGY | (SEQ ID NO:170) | 0.400 |
| 14 | 10 | ALLPAVPSL | (SEQ ID NO:34) | 0.400 |
| 15 | 207 | DSCTGSQAL | (SEQ ID NO:61) | 0.400 |
| 16 | 384 | QCKTCQRKF | (SEQ ID NO:163) | 0.400 |
| 17 | 136 | SCLESQPAI | (SEQ ID NO:198) | 0.300 |
| 18 | 347 | HTGEKPYQC | (SEQ ID NO:112) | 0.300 |
| 19 | 401 | HTRTHTGKT | (SEQ ID NO:114) | 0.200 |
| 20 | 332 | KRYFKLSHL | (SEQ ID NO:127) | 0.200 |

TABLE 19

RESULTS OF BIMAS HLA PEPTIDE BINDING PREDICTION ANALYSIS FOR BINDING OF
HUMAN WT1 PEPTIDES TO HUMAN HLA B 2702

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 332 | KRYFKLSHL | (SEQ ID NO:127) | 900.000 |
| 2 | 362 | RRFSRSDQL | (SEQ ID NO:187) | 900.000 |
| 3 | 286 | YRIHTHGVF | (SEQ ID NO:252) | 200.000 |
| 4 | 125 | ARMFPNAPY | (SEQ ID NO:38) | 200.000 |
| 5 | 375 | RRHTGVKPF | (SEQ ID NO:188) | 180.000 |
| 6 | 32 | AQWAPVLDF | (SEQ ID NO:37) | 100.000 |
| 7 | 301 | RRVPGVAPT | (SEQ ID NO:189) | 60.000 |
| 8 | 439 | QRNMTKLQL | (SEQ ID NO:173) | 60.000 |
| 9 | 126 | RMFPNAPYL | (SEQ ID NO:185) | 22.500 |
| 10 | 426 | ARSDELVRH | (SEQ ID NO:39) | 20.000 |
| 11 | 146 | NQGYSTVTF | (SEQ ID NO:150) | 20.000 |
| 12 | 144 | IRNQGYSTV | (SEQ ID NO:117) | 20.000 |
| 13 | 389 | QRKFSRSDH | (SEQ ID NO:172) | 20.000 |
| 14 | 263 | GQSNHSTGY | (SEQ ID NO:100) | 20.000 |
| 15 | 416 | CRWPSCQKK | (SEQ ID NO:50) | 20.000 |
| 16 | 191 | QQYSVPPPV | (SEQ ID NO:171) | 10.000 |
| 17 | 217 | LRTPYSSDN | (SEQ ID NO:140) | 10.000 |
| 18 | 107 | CRYGPFGPP | (SEQ ID NO:51) | 10.000 |
| 19 | 98 | GQFTGTAGA | (SEQ ID NO:99) | 10.000 |
| 20 | 239 | NQMNLGATL | (SEQ ID NO:151) | 6.000 |

TABLE 20

RESULTS OF BIMAS HLA PEPTIDE BINDING PREDICTION ANALYSIS FOR BINDING OF
HUMAN WT1 PEPTIDES TO HUMAN HLA B 2705

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 332 | KRYFKLSHL | (SEQ ID NO:127) | 30000.000 |
| 2 | 362 | RRFSRSDQL | (SEQ ID NO:187) | 30000.000 |
| 3 | 416 | CRWPSCQKK | (SEQ ID NO:50) | 10000.000 |
| 4 | 439 | QRNMTKLQL | (SEQ ID NO:173) | 2000.000 |
| 5 | 286 | YRIHTHGVF | (SEQ ID NO:252) | 1000.000 |
| 6 | 125 | ARMFPNAPY | (SEQ ID NO:38) | 1000.000 |
| 7 | 294 | FRGIQDVRR | (SEQ ID NO:81) | 1000.000 |
| 8 | 432 | VRHHNMHQR | (SEQ ID NO:243) | 1000.000 |
| 9 | 169 | AQFPNHSFK | (SEQ ID NO:36) | 1000.000 |
| 10 | 375 | RRHTGVKPF | (SEQ ID NO:188) | 900.000 |
| 11 | 126 | RMFPNAPYL | (SEQ ID NO:185) | 750.000 |
| 12 | 144 | IRNQGYSTV | (SEQ ID NO:117) | 600.000 |
| 13 | 301 | RRVPGVAPT | (SEQ ID NO:189) | 600.000 |
| 14 | 32 | AQWAPVLDF | (SEQ ID NO:37) | 500.000 |
| 15 | 191 | QQYSVPPPV | (SEQ ID NO:171) | 300.000 |
| 16 | 373 | HQRRHTGVK | (SEQ ID NO:109) | 200.000 |
| 17 | 426 | ARSDELVRH | (SEQ ID NO:39) | 200.000 |
| 18 | 383 | FQCKTCQRK | (SEQ ID NO:80) | 200.000 |
| 19 | 239 | NQMNLGATL | (SEQ ID NO:151) | 200.000 |
| 20 | 389 | QRKFSRSDH | (SEQ ID NO:172) | 200.000 |

TABLE 21
RESULTS OF BIMAS HLA PEPTIDE BINDING PREDICTION
ANALYSIS FOR BINDING OF HUMAN WT1 PEPTIDES TO
HUMAN HLA B 3501

| | | | | Score (Esti- |
|---|---|---|---|---|
| | | | | mate of Half |
| | | | | Time of Dis- |
| | | | | association |
| of a Molecule | | | | |
| | Start | | | Containing |
| | Posi- | Subsequence | | This |
| Rank | tion | Residue Listing | | Subsequence) |
| 1 | 278 | TPILCGAQY | (SEQ ID NO:227) | 40.000 |
| 2 | 141 | QPAIRNQGY | (SEQ ID NO:170) | 40.000 |
| 3 | 219 | TPYSSDNLY | (SEQ ID NO:231) | 40.000 |
| 4 | 327 | YPGCNKRYF | (SEQ ID NO:250) | 20.000 |
| 5 | 163 | TPSHHAAQF | (SEQ ID NO:228) | 20.000 |
| 6 | 180 | DPMGQQGSL | (SEQ ID NO:59) | 20.000 |
| 7 | 221 | YSSDNLYQM | (SEQ ID NO:253) | 20.000 |
| 8 | 26 | LPVSGAAQW | (SEQ ID NO:138) | 10.000 |
| 9 | 174 | HSFKHEDPM | (SEQ ID NO:110) | 10.000 |
| 10 | 82 | EPHEEQCLS | (SEQ ID NO:68) | 6.000 |
| 11 | 213 | QALLLRTPY | (SEQ ID NO:160) | 6.000 |
| 12 | 119 | QASSGQARM | (SEQ ID NO:161) | 6.000 |
| 13 | 4 | DVRDLNALL | (SEQ ID NO:62) | 6.000 |
| 14 | 40 | FAPPGASAY | (SEQ ID NO:74) | 6.000 |
| 15 | 120 | ASSGQARMF | (SEQ ID NO:40) | 5.000 |
| 16 | 207 | DSCTGSQAL | (SEQ ID NO:61) | 5.000 |
| 17 | 303 | VPGVAPTLV | (SEQ ID NO:242) | 4.000 |
| 18 | 316 | ETSEKRPFM | (SEQ ID NO:73) | 4.000 |
| 19 | 152 | VTFDGTPSY | (SEQ ID NO:244) | 4.000 |
| 20 | 412 | KPFSCRWPS | (SEQ ID NO:123) | 4.000 |

TABLE 22
RESULTS OF BIMAS HLA PEPTIDE BINDING PREDICTION
ANALYSIS FOR BINDING OF HUMAN WT1 PEPTIDES TO
HUMAN ULA B 3701

| | | | +HHC,24 Score (Esti- | |
|---|---|---|---|---|
| | | | mate of Half | |
| | | | Time of Dis- | |
| | | | association | |
| | | | of a Molecule | |
| | Start | | Containing | |
| | Posi- | Subsequence | This | |
| Rank | tion | Residue Listing | Subsequence) | |
| 1 | 3 | SDVRDLNAL | (SEQ ID NO:206) | 40.000 |
| 2 | 273 | SDNHTTPIL | (SEQ ID NO:204) | 40.000 |
| 3 | 81 | AEPHEEQCL | (SEQ ID NO:30) | 10.000 |
| 4 | 298 | QDVRRVPGV | (SEQ ID NO:164) | 8.000 |
| 5 | 428 | SDELVRHHN | (SEQ ID NO:203) | 6.000 |
| 6 | 85 | EEQCLSAFT | (SEQ ID NO:65) | 5.000 |
| 7 | 208 | SCTGSQALL | (SEQ ID NO:202) | 5.000 |
| 8 | 4 | DVRDLNALL | (SEQ ID NO:62) | 5.000 |
| 9 | 209 | CTGSQALLL | (SEQ ID NO:52) | 5.000 |
| 10 | 38 | LDFAPPGAS | (SEQ ID NO:130) | 4.000 |
| 11 | 223 | SDNLYQMTS | (SEQ ID NO:205) | 4.000 |
| 12 | 179 | EDPMGQQGS | (SEQ ID NO:64) | 4.000 |
| 13 | 206 | TDSCTGSQA | (SEQ ID NO:220) | 4.000 |
| 14 | 6 | RDLNALLPA | (SEQ ID NO:177) | 4.000 |
| 15 | 84 | HEEQCLSAF | (SEQ ID NO:107) | 2.000 |
| 16 | 233 | LECMTWNQM | (SEQ ID NO:131) | 2.000 |
| 17 | 429 | DELVRHHNM | (SEQ ID NO:53) | 2.000 |
| 18 | 315 | SETSEKRPF | (SEQ ID NO:209) | 2.000 |
| 19 | 349 | GEKPYQCDF | (SEQ ID NO:91) | 2.000 |
| 20 | 302 | RVPGVAPTL | (SEQ ID NO:195) | 1.500 |

TABLE 23
RESULTS OF BIMAS HLA PEPTIDE BINDING PREDICTION
ANALYSIS FOR BINDING OF HUMAN WT1 PEPTIDES TO
HUMAN HLA B 3801

| | | | | Score (Esti- |
|---|---|---|---|---|
| | | | | mate of Half |
| | | | | Time of Dis- |
| | | | | association |
| | | | | of a Molecule |
| | Start | | | Containing |
| | Posi- | Subsequence | | This |
| Rank | tion | Residue Listing | | Subsequence) |
| 1 | 437 | MHQRNMTKL | (SEQ ID NO:143) | 36.000 |
| 2 | 434 | HHNMHQRNM | (SEQ ID NO:108) | 6.000 |
| 3 | 372 | RHQRRHTGV | (SEQ ID NO:181) | 6.000 |
| 4 | 180 | DPMGQQGSL | (SEQ ID NO:59) | 4.000 |
| 5 | 433 | RHHNMHQRN | (SEQ ID NO:180) | 3.900 |
| 6 | 165 | SHHAAQFPN | (SEQ ID NO:213) | 3.900 |
| 7 | 202 | CHTPTDSCT | (SEQ ID NO:45) | 3.000 |
| 8 | 396 | DHLKTHTRT | (SEQ ID NO:57) | 3.000 |
| 9 | 161 | GHTPSHHAA | (SEQ ID NO:94) | 3.000 |
| 10 | 302 | RVPGVAPTL | (SEQ ID NO:195) | 2.600 |
| 11 | 417 | RWPSCQKKF | (SEQ ID NO:196) | 2.400 |
| 12 | 327 | YPGCNKRYF | (SEQ ID NO:250) | 2.400 |
| 13 | 208 | SCTGSQALL | (SEQ ID NO:202) | 2.000 |
| 14 | 163 | TPSHHAAQF | (SEQ ID NO:228) | 2.000 |
| 15 | 120 | ASSGQARMF | (SEQ ID NO:40) | 2.000 |
| 16 | 18 | LGGGGGCAL | (SEQ ID NO:134) | 2.000 |
| 17 | 177 | KHEDPMGQQ | (SEQ ID NO:121) | 1.800 |
| 18 | 83 | PHEEQCLSA | (SEQ ID NO:154) | 1.800 |
| 19 | 10 | ALLPAVPSL | (SEQ ID NO:34) | 1.300 |
| 20 | 225 | NLYQMTSQL | (SEQ ID NO:147) | 1.300 |

TABLE 24
RESULTS OF BIMAS HLA PEPTIDE BINDING PREDICTION
ANALYSIS FOR BINDING OF HUMAN WT1 PEPTIDES TO
HUMAN HLA B 3901

| | | | | Score (Esti- |
|---|---|---|---|---|
| | | | | mate of Half |
| | | | | Time of Dis- |
| | | | | association |
| | | | | of a Molecule |
| | Start | | | Containing |
| | Posi- | Subsequence | | This |
| Rank | tion | Residue Listing | | Subsequence) |
| 1 | 437 | MHQRNMTKL | (SEQ ID NO:143) | 135.000 |
| 2 | 332 | KRYFKLSHL | (SEQ ID NO:127) | 45.000 |
| 3 | 434 | HHNMHQRNM | (SEQ ID NO:108) | 30.000 |
| 4 | 362 | RRFSRSDQL | (SEQ ID NO:187) | 30.000 |
| 5 | 372 | RHQRRHTGV | (SEQ ID NO:181) | 30.000 |
| 6 | 10 | ALLPAVPSL | (SEQ ID NO:34) | 9.000 |
| 7 | 439 | QRNMTKLQL | (SEQ ID NO:173) | 7.500 |
| 8 | 390 | RKFSRSDHL | (SEQ ID NO:183) | 6.000 |
| 9 | 396 | DHLKTHTRT | (SEQ ID NO:57) | 6.000 |
| 10 | 239 | NQMNLGATL | (SEQ ID NO:151) | 6.000 |
| 11 | 423 | KKFARSDEL | (SEQ ID NO:122) | 6.000 |
| 12 | 126 | RMFPNAPYL | (SEQ ID NO:185) | 6.000 |
| 13 | 225 | NLYQMTSQL | (SEQ ID NO:147) | 6.000 |
| 14 | 180 | DPMGQQGSL | (SEQ ID NO:59) | 6.000 |
| 15 | 144 | IRNQGYSTV | (SEQ ID NO:117) | 5.000 |
| 16 | 136 | SCLESQPAI | (SEQ ID NO:198) | 4.000 |
| 17 | 292 | GVFRGIQDV | (SEQ ID NO:103) | 3.000 |
| 18 | 302 | RVPGVAPTL | (SEQ ID NO:195) | 3.000 |
| 19 | 208 | SCTGSQALL | (SEQ ID NO:202) | 3.000 |
| 20 | 207 | DSCTGSQAL | (SEQ ID NO:61) | 3.000 |

TABLE 25
RESULTS OF BIMAS HLA PEPTIDE BINDING PREDICTION
ANALYSIS FOR BINDING OF HUMAN WT1 PEPTIDES TO
HUMAN ULA B 3902

| | | | | Score (Esti- |
|---|---|---|---|---|
| | | | | mate of Half |
| | | | | Time of Dis- |
| | | | | association |
| | | | | of a Molecule |
| | Start | | | Containing |
| | Posi- | Subsequence | | This |
| Rank | tion | Residue Listing | | Subsequence) |
| 1 | 239 | NQMNLGATL | (SEQ ID NO:151) | 24.000 |
| 2 | 390 | RKFSRSDHL | (SEQ ID NO:183) | 20.000 |
| 3 | 423 | KKFARSDEL | (SEQ ID NO:122) | 20.000 |
| 4 | 32 | AQWAPVLDF | (SEQ ID NO:37) | 5.000 |
| 5 | 146 | NQGYSTVTF | (SEQ ID NO:150) | 5.000 |
| 6 | 130 | NAPYLPSCL | (SEQ ID NO:144) | 2.400 |
| 7 | 225 | NLYQMTSQL | (SEQ ID NO:147) | 2.400 |
| 8 | 30 | GAAQWAPVL | (SEQ ID NO:86) | 2.400 |
| 9 | 441 | NMTKLQLAL | (SEQ ID NO:149) | 2.400 |
| 10 | 302 | RVPGVAPTL | (SEQ ID NO:195) | 2.400 |
| 11 | 126 | RMFPNAPYL | (SEQ ID NO:185) | 2.000 |
| 12 | 218 | RTPYSSDNL | (SEQ ID NO:194) | 2.000 |
| 13 | 209 | CTGSQALLL | (SEQ ID NO:52) | 2.000 |
| 14 | 332 | KRYFKLSHL | (SEQ ID NO:127) | 2.000 |
| 15 | 180 | DPMGQQGSL | (SEQ ID NO:59) | 2.000 |
| 16 | 437 | MHQRNMTKL | (SEQ ID NO:143) | 2.000 |
| 17 | 207 | DSCTGSQAL | (SEQ ID NO:61) | 2.000 |
| 18 | 208 | SCTGSQALL | (SEQ ID NO:202) | 2.000 |
| 19 | 329 | GCNKRYFKL | (SEQ ID NO:90) | 2.000 |
| 20 | 10 | ALLPAVPSL | (SEQ ID NO:34) | 2.000 |

TABLE 26
RESULTS OF BIMAS HLA PEPTIDE BINDING PREDICTION
ANALYSIS FOR BINDING OF HUMAN WT1 PEPTIDES TO
HUMAN HLA B 4403

| | | | | Score (Esti- |
|---|---|---|---|---|
| | | | | mate of Half |
| | | | | Time of Dis- |
| | | | | association |
| | | | | of a Molecule |
| | Start | | | Containing |
| | Posi- | Subsequence | | This |
| Rank | tion | Residue Listing | | Subsequence) |
| 1 | 315 | SETSEKRPF | (SEQ ID NO:209) | 80.000 |
| 2 | 349 | GEKPYQCDF | (SEQ ID NO:91) | 80.000 |
| 3 | 84 | HEEQCLSAF | (SEQ ID NO:107) | 60.000 |
| 4 | 410 | SEKPFSCRW | (SEQ ID NO:207) | 48.000 |
| 5 | 429 | DELVRHHNM | (SEQ ID NO:53) | 24.000 |
| 6 | 278 | TPILCGAQY | (SEQ ID NO:227) | 15.000 |
| 7 | 141 | QPAIRNQGY | (SEQ ID NO:170) | 9.000 |
| 8 | 40 | FAPPGASAY | (SEQ ID NO:74) | 9.000 |
| 9 | 213 | QALLLRTPY | (SEQ ID NO:160) | 9.000 |
| 10 | 318 | SEKRPFMCA | (SEQ ID NO:208) | 8.000 |
| 11 | 81 | AEPHEEQCL | (SEQ ID NO:30) | 8.000 |
| 12 | 152 | VTFDGTPSY | (SEQ ID NO:244) | 4.500 |
| 13 | 101 | TGTAGACRY | (SEQ ID NO:224) | 4.500 |
| 14 | 120 | ASSGQARMF | (SEQ ID NO:40) | 4.500 |
| 15 | 261 | TEGQSNHST | (SEQ ID NO:221) | 4.000 |
| 16 | 85 | EEQCLSAFT | (SEQ ID NO:65) | 4.000 |
| 17 | 233 | LECMTWNQM | (SEQ ID NO:131) | 4.000 |
| 18 | 104 | AGACRYGPF | (SEQ ID NO:31) | 4.000 |
| 19 | 3 | SDVRDLNAL | (SEQ ID NO:206) | 3.000 |
| 20 | 185 | QGSLGEQQY | (SEQ ID NO:166) | 3.000 |

TABLE 27
RESULTS OF BIMAS ULA PEPTIDE BINDING PREDICTION
ANALYSIS FOR BINDING OF HUMAN WT1 PEPTIDES TO
HUMAN HLA B 5101

| | | | | Score (Esti- |
|---|---|---|---|---|
| | | | | mate of Half |
| | | | | Time of Dis- |
| | | | | association |
| | | | | of a Molecule |
| | Start | | | Containing |
| | Posi- | Subsequence | | This |
| Rank | tion | Residue Listing | | Subsequence) |
| 1 | 303 | VPGVAPTLV | (SEQ ID NO:242) | 314.600 |
| 2 | 180 | DPMGQQGSL | (SEQ ID NO:59) | 242.000 |
| 3 | 250 | VAAGSSSSV | (SEQ ID NO:236) | 157.300 |
| 4 | 130 | NAPYLPSCL | (SEQ ID NO:144) | 50.000 |
| 5 | 30 | GAAQWAPVL | (SEQ ID NO:86) | 50.000 |
| 6 | 20 | GGGGCALPV | (SEQ ID NO:92) | 44.000 |
| 7 | 64 | PPPPPHSFI | (SEQ ID NO:157) | 40.000 |
| 8 | 29 | SGAAQWAPV | (SEQ ID NO:211) | 40.000 |
| 9 | 18 | LGGGGGCAL | (SEQ ID NO:134) | 31.460 |
| 10 | 295 | RGIQDVRRV | (SEQ ID NO:179) | 22.000 |
| 11 | 119 | QASSGQARM | (SEQ ID NO:161) | 18.150 |
| 12 | 418 | WPSCQKKFA | (SEQ ID NO:246) | 12.100 |
| 13 | 82 | EPHEEQCLS | (SEQ ID NO:68) | 12.100 |
| 14 | 110 | GPFGPPPPS | (SEQ ID NO:96) | 11.000 |
| 15 | 272 | ESDNHTTPI | (SEQ ID NO:71) | 8.000 |
| 16 | 306 | VAPTLVRSA | (SEQ ID NO:237) | 7.150 |
| 17 | 280 | ILCGAQYRI | (SEQ ID NO:116) | 6.921 |
| 18 | 219 | TPYSSDNLY | (SEQ ID NO:231) | 6.600 |
| 19 | 128 | FPNAPYLPS | (SEQ ID NO:79) | 6.500 |
| 20 | 204 | TPTDSCTGS | (SEQ ID NO:230) | 6.050 |

TABLE 28
RESULTS OF BIMAS HLA PEPTIDE BINDING PREDICTION
ANALYSIS FORBINDING OF HUMAN WT1 PEPTIDES TO
HUMAN HLA B 5102

| | | | | Score (Esti- |
|---|---|---|---|---|
| | | | | mate of Half |
| | | | | Time of Dis- |
| | | | | association |
| | | | | of a Molecule |
| | Start | | | Containing |
| | Posi- | Subsequence | | This |
| Rank | tion | Residue Listing | | Subsequence) |
| 1 | 295 | RGIQDVRRV | (SEQ ID NO:179) | 290.400 |
| 2 | 303 | VPGVAPTLV | (SEQ ID NO:242) | 200.000 |
| 3 | 180 | DPMGQQGSL | (SEQ ID NO:59) | 133.100 |
| 4 | 250 | VAAGSSSSV | (SEQ ID NO:236) | 110.000 |
| 5 | 30 | GAAQWAPVL | (SEQ ID NO:86) | 55.000 |
| 6 | 130 | NAPYLPSCL | (SEQ ID NO:144) | 50.000 |
| 7 | 20 | GGGGCALPV | (SEQ ID NO:92) | 44.000 |
| 8 | 29 | SGAAQWAPV | (SEQ ID NO:211) | 44.000 |
| 9 | 64 | PPPPPHSFI | (SEQ ID NO:157) | 40.000 |
| 10 | 119 | QASSGQARM | (SEQ ID NO:161) | 36.300 |
| 11 | 110 | GPFGPPPPS | (SEQ ID NO:96) | 27.500 |
| 12 | 412 | KPFSCRWPS | (SEQ ID NO:123) | 25.000 |
| 13 | 18 | LGGGGGCAL | (SEQ ID NO:134) | 24.200 |
| 14 | 24 | CALPVSGAA | (SEQ ID NO:43) | 16.500 |
| 15 | 219 | TPYSSDNLY | (SEQ ID NO:231) | 15.000 |
| 16 | 292 | GVFRGIQDV | (SEQ ID NO:103) | 14.641 |
| 17 | 136 | SCLESQPAI | (SEQ ID NO:198) | 14.520 |
| 18 | 418 | WPSCQKKFA | (SEQ ID NO:246) | 12.100 |
| 19 | 269 | TGYESDNHT | (SEQ ID NO:225) | 11.000 |
| 20 | 351 | KPYQCDFKD | (SEQ ID NO:124) | 11.000 |

TABLE 29
RESULTS OF BIMAS HLA PEPTIDE BINDING PREDICTION
ANALYSIS FOR BINDING OF HUMAN WT1 PEPTIDES TO
HUMAN HLA B 5201

| | | | | Score (Esti- |
|---|---|---|---|---|
| | | | | mate of Half |
| | | | | Time of Dis- |
| | | | | association |
| | | | | of a Molecule |
| | Start | | | Containing |
| | Posi- | Subsequence | | This |
| Rank | tion | Residue Listing | | Subsequence) |
| 1 | 191 | QQYSVPPPV | (SEQ ID NO:171) | 100.000 |
| 2 | 32 | AQWAPVLDF | (SEQ ID NO:37) | 30.000 |
| 3 | 243 | LGATLKGVA | (SEQ ID NO:133) | 16.500 |
| 4 | 303 | VPGVAPTLV | (SEQ ID NO:242) | 13.500 |
| 5 | 86 | EQCLSAFTV | (SEQ ID NO:69) | 12.000 |
| 6 | 295 | RGIQDVRRV | (SEQ ID NO:179) | 10.000 |
| 7 | 98 | GQFTGTAGA | (SEQ ID NO:99) | 8.250 |
| 8 | 292 | GVFRGIQDV | (SEQ ID NO:103) | 8.250 |
| 9 | 29 | SGAAQWAPV | (SEQ ID NO:211) | 6.000 |
| 10 | 146 | NQGYSTVTF | (SEQ ID NO:150) | 5.500 |
| 11 | 20 | GGGGCALPV | (SEQ ID NO:92) | 5.000 |
| 12 | 239 | NQMNLGATL | (SEQ ID NO:151) | 4.000 |
| 13 | 64 | PPPPPHSFI | (SEQ ID NO:157) | 3.600 |
| 14 | 273 | SDNHTTPIL | (SEQ ID NO:204) | 3.300 |
| 15 | 286 | YRIHTHGVF | (SEQ ID NO:252) | 3.000 |
| 16 | 269 | TGYESDNHT | (SEQ ID NO:225) | 3.000 |
| 17 | 406 | TGKTSEKPF | (SEQ ID NO:222) | 2.750 |
| 18 | 327 | YPGCNKRYF | (SEQ ID NO:250) | 2.750 |
| 19 | 7 | DLNALLPAV | (SEQ ID NO:58) | 2.640 |
| 20 | 104 | AGACRYGPF | (SEQ ID NO:31) | 2.500 |

TABLE 30
RESULTS OF BIMAS HLA PEPTIDE BINDING PREDICTION
ANALYSIS FOR BINDING OF HUMAN WT1 PEPTIDES TO
HUMAN HLA B 5801

| | | | | Score (Esti- |
|---|---|---|---|---|
| | | | | mate of Half |
| | | | | Time of Dis- |
| | | | | association |
| | | | | of a Molecule |
| | Start | | | Containing |
| | Posi- | Subsequence | | This |
| Rank | tion | Residue Listing | | Subsequence) |
| 1 | 230 | TSQLECMTW | (SEQ ID NO:234) | 96.800 |
| 2 | 92 | FTVHFSGQF | (SEQ ID NO:85) | 60.000 |
| 3 | 120 | ASSGQARMF | (SEQ ID NO:40) | 40.000 |
| 4 | 168 | AAQFPNHSF | (SEQ ID NO:29) | 20.000 |
| 5 | 408 | KTSEKPFSC | (SEQ ID NO:129) | 12.000 |
| 6 | 394 | RSDHLKTHT | (SEQ ID NO:192) | 9.900 |
| 7 | 276 | HTTPILCGA | (SEQ ID NO:115) | 7.200 |
| 8 | 218 | RTPYSSDNL | (SEQ ID NO:194) | 6.600 |
| 9 | 152 | VTFDGTPSY | (SEQ ID NO:244) | 6.000 |
| 10 | 40 | FAPPGASAY | (SEQ ID NO:74) | 6.000 |
| 11 | 213 | QALLLRTPY | (SEQ ID NO:160) | 4.500 |
| 12 | 347 | HTGEKPYQC | (SEQ ID NO:112) | 4.400 |
| 13 | 252 | AGSSSSVKW | (SEQ ID NO:32) | 4.400 |
| 14 | 211 | GSQALLLRT | (SEQ ID NO:102) | 4.356 |
| 15 | 174 | HSFKHEDPM | (SEQ ID NO:110) | 4.000 |
| 16 | 317 | TSEKRPFMC | (SEQ ID NO:233) | 4.000 |
| 17 | 26 | LPVSGAAQW | (SEQ ID NO:138) | 4.000 |
| 18 | 289 | HTHGVFRGI | (SEQ ID NO:113) | 3.600 |
| 19 | 222 | SSDNLYQMT | (SEQ ID NO:217) | 3.300 |
| 20 | 96 | FSGQFTGTA | (SEQ ID NO:82) | 3.300 |

TABLE 31

RESULTS OF BIMAS HLA PEPTIDE BINDING PREDICTION ANALYSIS FOR BINDING OF
HUMAN WT1 PEPTIDES TO HUMAN HLA CW0301

| | | | |
|---|---|---|---|
| | | | Score (Estimate of Half Time |
| | Start | | Disassociation of a Molecule |
| Rank | Position | Subsequence Residue Listing | Containing This Subsequence) |

| | | | | |
|---|---|---|---|---|
| 1 | 10 | ALLPAVPSL | (SEQ ID NO:34) | 100.000 |
| 2 | 332 | KRYFKLSHL | (SEQ ID NO:127) | 48.000 |
| 3 | 126 | RMFPNAPYL | (SEQ ID NO:185) | 36.000 |
| 4 | 3 | SDVRDLNAL | (SEQ ID NO:206) | 30.000 |
| 5 | 239 | NQMNLGATL | (SEQ ID NO:151) | 24.000 |
| 6 | 225 | NLYQMTSQL | (SEQ ID NO:147) | 24.000 |
| 7 | 180 | DPMGQQGSL | (SEQ ID NO:59) | 20.000 |
| 8 | 362 | RRFSRSDQL | (SEQ ID NO:187) | 12.000 |
| 9 | 329 | GCNKRYFKL | (SEQ ID NO:90) | 10.000 |
| 10 | 286 | YRIHTHGVF | (SEQ ID NO:252) | 10.000 |
| 11 | 301 | RRVPGVAPT | (SEQ ID NO:189) | 10.000 |
| 12 | 24 | CALPVSGAA | (SEQ ID NO:43) | 10.000 |
| 13 | 136 | SCLESQPAI | (SEQ ID NO:198) | 7.500 |
| 14 | 437 | MHQRNMTKL | (SEQ ID NO:143) | 7.200 |
| 15 | 390 | RKFSRSDHL | (SEQ ID NO:183) | 6.000 |
| 16 | 423 | KKFARSDEL | (SEQ ID NO:122) | 6.000 |
| 17 | 92 | FTVHFSGQF | (SEQ ID NO:85) | 5.000 |
| 18 | 429 | DELVRHHNM | (SEQ ID NO:53) | 5.000 |
| 19 | 130 | NAPYLPSCL | (SEQ ID NO:144) | 4.800 |
| 20 | 30 | GAAQWAPVL | (SEQ ID NO:86) | 4.000 |

TABLE 32

RESULTS OF BIMAS HLA PEPTIDE BINDING PREDICTION ANALYSIS FOR BINDING OF
HUMAN WT1 PEPTIDES TO HUMAN HLA CW0401

| | | | |
|---|---|---|---|
| | | | Score (Estimate of Half Time |
| | Start | | Disassociation of a Molecule |
| Rank | Position | Subsequence Residue Listing | Containing This Subsequence) |

| | | | | |
|---|---|---|---|---|
| 1 | 356 | DFKDCERRF | (SEQ ID NO:55) | 120.000 |
| 2 | 334 | YFKLSHLQM | (SEQ ID NO:248) | 100.000 |
| 3 | 180 | DPMGQQGSL | (SEQ ID NO:59) | 88.000 |
| 4 | 163 | TPSHHAAQF | (SEQ ID NO:228) | 52.800 |
| 5 | 327 | YPGCNKRYF | (SEQ ID NO:250) | 40.000 |
| 6 | 285 | QYRIHTHGV | (SEQ ID NO:175) | 27.500 |
| 7 | 424 | KFARSDELV | (SEQ ID NO:119) | 25.000 |
| 8 | 326 | AYPGCNKRY | (SEQ ID NO:42) | 25.000 |
| 9 | 192 | QYSVPPPVY | (SEQ ID NO:176) | 25.000 |
| 10 | 417 | RWPSCQKKF | (SEQ ID NO:196) | 22.000 |
| 11 | 278 | TPILCGAQY | (SEQ ID NO:227) | 12.000 |
| 12 | 10 | ALLPAVPSL | (SEQ ID NO:34) | 11.616 |
| 13 | 141 | QPAIRNQGY | (SEQ ID NO:170) | 11.000 |
| 14 | 303 | VPGVAPTLV | (SEQ ID NO:242) | 11.000 |
| 15 | 219 | TPYSSDNLY | (SEQ ID NO:231) | 10.000 |
| 16 | 39 | DFAPPGASA | (SEQ ID NO:54) | 7.920 |
| 17 | 99 | QFTGTAGAC | (SEQ ID NO:165) | 6.000 |
| 18 | 4 | DVRDLNALL | (SEQ ID NO:62) | 5.760 |
| 19 | 70 | SFIKQEPSW | (SEQ ID NO:210) | 5.500 |
| 20 | 63 | PPPPPPHSF | (SEQ ID NO:158) | 5.280 |

TABLE 33

RESULTS OF BIMAS HLA PEPTIDE BINDING PREDICTION ANALYSIS FOR BINDING OF
HUMAN WT1 PEPTIDES TO HUMAN HLA CW0602

| | | | |
|---|---|---|---|
| | | | Score (Estimate of Half Time |
| | Start | | Disassociation of a Molecule |
| Rank | Position | Subsequence Residue Listing | Containing This Subsequence) |

| | | | | |
|---|---|---|---|---|
| 1 | 332 | KRYFKLSHL | (SEQ ID NO:127) | 9.680 |
| 2 | 239 | NQMNLGATL | (SEQ ID NO:151) | 6.600 |
| 3 | 130 | NAPYLPSCL | (SEQ ID NO:144) | 6.600 |
| 4 | 7 | DLNALLPAV | (SEQ ID NO:58) | 6.000 |
| 5 | 441 | NMTKLQLAL | (SEQ ID NO:149) | 6.000 |
| 6 | 225 | NLYQMTSQL | (SEQ ID NO:147) | 6.000 |
| 7 | 4 | DVRDLNALL | (SEQ ID NO:62) | 6.000 |
| 8 | 3 | SDVRDLNAL | (SEQ ID NO:206) | 4.400 |
| 9 | 10 | ALLPAVPSL | (SEQ ID NO:34) | 4.000 |
| 10 | 213 | QALLLRTPY | (SEQ ID NO:160) | 3.300 |
| 11 | 319 | EKRPFMCAY | (SEQ ID NO:67) | 3.000 |
| 12 | 30 | GAAQWAPVL | (SEQ ID NO:86) | 2.200 |
| 13 | 242 | NLGATLKGV | (SEQ ID NO:146) | 2.200 |
| 14 | 292 | GVFRGIQDV | (SEQ ID NO:103) | 2.200 |
| 15 | 207 | DSCTGSQAL | (SEQ ID NO:61) | 2.200 |
| 16 | 362 | RRFSRSDQL | (SEQ ID NO:187) | 2.200 |
| 17 | 439 | QRNMTKLQL | (SEQ ID NO:173) | 2.200 |
| 18 | 295 | RGIQDVRRV | (SEQ ID NO:179) | 2.200 |
| 19 | 423 | KKFARSDEL | (SEQ ID NO:122) | 2.200 |
| 20 | 180 | DPMGQQGSL | (SEQ ID NO:59) | 2.200 |

TABLE 34

RESULTS OF BIMAS HLA PEPTIDE BINDING PREDICTION ANALYSIS FOR BINDING OF
HUMAN WT1 PEPTIDES TO HUMAN HLA CW0702

| | | | |
|---|---|---|---|
| | | | Score (Estimate of Half Time |
| | Start | | Disassociation of a Molecule |
| Rank | Position | Subsequence Residue Listing | Containing This Subsequence) |

| | | | | |
|---|---|---|---|---|
| 1 | 319 | EKRPFMCAY | (SEQ ID NO:67) | 26.880 |
| 2 | 326 | AYPGCNKRY | (SEQ ID NO:42) | 24.000 |
| 3 | 40 | FAPPGASAY | (SEQ ID NO:74) | 14.784 |
| 4 | 192 | QYSVPPPVY | (SEQ ID NO:176) | 12.000 |
| 5 | 278 | TPILCGAQY | (SEQ ID NO:227) | 12.000 |
| 6 | 219 | TPYSSDNLY | (SEQ ID NO:231) | 12.000 |
| 7 | 213 | QALLLRTPY | (SEQ ID NO:160) | 8.800 |
| 8 | 125 | ARMFPNAPY | (SEQ ID NO:38) | 8.000 |
| 9 | 327 | YPGCNKRYF | (SEQ ID NO:250) | 6.600 |
| 10 | 152 | VTFDGTPSY | (SEQ ID NO:244) | 5.600 |
| 11 | 141 | QPAIRNQGY | (SEQ ID NO:170) | 4.800 |
| 12 | 345 | RKHTGEKPY | (SEQ ID NO:184) | 4.000 |
| 13 | 185 | QGSLGEQQY | (SEQ ID NO:166) | 4.000 |
| 14 | 101 | TGTAGACRY | (SEQ ID NO:224) | 4.000 |
| 15 | 375 | RRHTGVKPF | (SEQ ID NO:188) | 4.000 |
| 16 | 263 | GQSNHSTGY | (SEQ ID NO:100) | 4.000 |
| 17 | 163 | TPSHHAAQF | (SEQ ID NO:228) | 3.000 |
| 18 | 33 | QWAPVLDFA | (SEQ ID NO:174) | 2.688 |
| 19 | 130 | NAPYLPSCL | (SEQ ID NO:144) | 2.640 |
| 20 | 84 | HEEQCLSAF | (SEQ ID NO:107) | 2.400 |

TABLE 35

RESULTS OF BIMAS HLA PEPTIDE BINDING PREDICTION ANALYSIS FOR BINDING OF
HUMAN WT1 PEPTIDES TO MOUSE MHC CLASS I DB

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 235 | CMTWNQMNL | (SEQ ID NO:49) | 5255.712 |
| 2 | 126 | RMFPNAPYL | (SEQ ID NO:185) | 1990.800 |
| 3 | 221 | YSSDNLYQM | (SEQ ID NO:253) | 930.000 |
| 4 | 228 | QMTSQLECM | (SEQ ID NO:169) | 33.701 |
| 5 | 239 | NQMNLGATL | (SEQ ID NO:151) | 21.470 |
| 6 | 441 | NMTKLQLAL | (SEQ ID NO:149) | 19.908 |
| 7 | 437 | MHQRNMTKL | (SEQ ID NO:143) | 19.837 |
| 8 | 136 | SCLESQPAI | (SEQ ID NO:198) | 11.177 |
| 9 | 174 | HSFKHEDPM | (SEQ ID NO:110) | 10.800 |
| 10 | 302 | RVPGVAPTL | (SEQ ID NO:195) | 10.088 |
| 11 | 130 | NAPYLPSCL | (SEQ ID NO:144) | 8.400 |
| 12 | 10 | ALLPAVPSL | (SEQ ID NO:34) | 5.988 |
| 13 | 208 | SCTGSQALL | (SEQ ID NO:202) | 4.435 |
| 14 | 209 | CTGSQALLL | (SEQ ID NO:52) | 3.548 |
| 15 | 238 | WNQMNLGAT | (SEQ ID NO:245) | 3.300 |
| 16 | 218 | RTPYSSDNL | (SEQ ID NO:194) | 3.185 |
| 17 | 24 | CALPVSGAA | (SEQ ID NO:43) | 2.851 |
| 18 | 18 | LGGGGGCAL | (SEQ ID NO:134) | 2.177 |
| 19 | 142 | PAIRNQGYS | (SEQ ID NO:152) | 2.160 |
| 20 | 30 | GAAQWAPVL | (SEQ ID NO:86) | 1.680 |

TABLE 36

RESULTS OF BIMAS HLA PEPTIDE BINDING PREDICTION ANALYSIS FOR BINDING OF
HUMAN WT1 PEPTIDES TO MOUSE MHC CLASS I DD

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 112 | FGPPPPSQA | (SEQ ID NO:76) | 48.000 |
| 2 | 122 | SGQARMFPN | (SEQ ID NO:212) | 36.000 |
| 3 | 104 | AGACRYGPF | (SEQ ID NO:31) | 30.000 |
| 4 | 218 | RTPYSSDNL | (SEQ ID NO:194) | 28.800 |
| 5 | 130 | NAPYLPSCL | (SEQ ID NO:144) | 20.000 |
| 6 | 302 | RVPGVAPTL | (SEQ ID NO:195) | 20.000 |
| 7 | 18 | LGGGGGCAL | (SEQ ID NO:134) | 20.000 |
| 8 | 81 | AEPHEEQCL | (SEQ ID NO:30) | 10.000 |
| 9 | 29 | SGAAQWAPV | (SEQ ID NO:211) | 7.200 |
| 10 | 423 | KKFARSDEL | (SEQ ID NO:122) | 7.200 |
| 11 | 295 | RGIQDVRRV | (SEQ ID NO:179) | 7.200 |
| 12 | 390 | RKFSRSDHL | (SEQ ID NO:183) | 6.000 |
| 13 | 332 | KRYFKLSHL | (SEQ ID NO:127) | 6.000 |
| 14 | 362 | RRFSRSDQL | (SEQ ID NO:187) | 6.000 |
| 15 | 417 | RWPSCQKKF | (SEQ ID NO:196) | 6.000 |
| 16 | 160 | YGHTPSHHA | (SEQ ID NO:249) | 6.000 |
| 17 | 20 | GGGGCALPV | (SEQ ID NO:92) | 6.000 |
| 18 | 329 | GCNKRYFKL | (SEQ ID NO:90) | 5.000 |
| 19 | 372 | RHQRRHTGV | (SEQ ID NO:181) | 4.500 |
| 20 | 52 | GGPAPPPAP | (SEQ ID NO:93) | 4.000 |

TABLE 37

RESULTS OF BIMAS HLA PEPTIDE BINDING PREDICTION ANALYSIS FOR BINDING OF
HUMAN WT1 PEPTIDES TO MOUSE MHC CLASS I KB

| | | | |
|---|---|---|---|
| | | | Score (Estimate of Half Time |
| | Start | | Disassociation of a Molecule |
| Rank | Position | Subsequence Residue Listing | Containing This Subsequence) |

| | | | | |
|---|---|---|---|---|
| 1 | 329 | GCNKRYFKL | (SEQ ID NO:90) | 24.000 |
| 2 | 225 | NLYQMTSQL | (SEQ ID NO:147) | 10.000 |
| 3 | 420 | SCQKKFARS | (SEQ ID NO:200) | 3.960 |
| 4 | 218 | RTPYSSDNL | (SEQ ID NO:194) | 3.630 |
| 5 | 437 | MHQRNMTKL | (SEQ ID NO:143) | 3.600 |
| 6 | 387 | TCQRKFSRS | (SEQ ID NO:219) | 3.600 |
| 7 | 302 | RVPGVAPTL | (SEQ ID NO:195) | 3.300 |
| 8 | 130 | NAPYLPSCL | (SEQ ID NO:144) | 3.000 |
| 9 | 289 | HTHGVFRGI | (SEQ ID NO:113) | 3.000 |
| 10 | 43 | PGASAYGSL | (SEQ ID NO:153) | 2.400 |
| 11 | 155 | DGTPSYGHT | (SEQ ID NO:56) | 2.400 |
| 12 | 273 | SDNHTTPIL | (SEQ ID NO:204) | 2.200 |
| 13 | 126 | RMFPNAPYL | (SEQ ID NO:185) | 2.200 |
| 14 | 128 | FPNAPYLPS | (SEQ ID NO:79) | 2.000 |
| 15 | 3 | SDVRDLNAL | (SEQ ID NO:206) | 1.584 |
| 16 | 207 | DSCTGSQAL | (SEQ ID NO:61) | 1.584 |
| 17 | 332 | KRYFKLSHL | (SEQ ID NO:127) | 1.500 |
| 18 | 18 | LGGGGGCAL | (SEQ ID NO:134) | 1.320 |
| 19 | 233 | LECMTWNQM | (SEQ ID NO:131) | 1.320 |
| 20 | 441 | NMTKLQLAL | (SEQ ID NO:149) | 1.200 |

TABLE 38

RESULTS OF BIMAS HLA PEPTIDE BINDING PREDICTION ANALYSIS FOR BINDING OF
HUMAN WT1 PEPTIDES TO MOUSE MHC CLASS I KD

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 285 | QYRIHTHGV | (SEQ ID NO:175) | 600.000 |
| 2 | 424 | KFARSDELV | (SEQ ID NO:119) | 288.000 |
| 3 | 334 | YFKLSHLQM | (SEQ ID NO:248) | 120.000 |
| 4 | 136 | SCLESQPTI | (SEQ ID NO:199) | 115.200 |
| 5 | 239 | NQMNLGATL | (SEQ ID NO:151) | 115.200 |
| 6 | 10 | ALLPAVSSL | (SEQ ID NO:35) | 115.200 |
| 7 | 47 | AYGSLGGPA | (SEQ ID NO:41) | 86.400 |
| 8 | 180 | DPMGQQGSL | (SEQ ID NO:59) | 80.000 |
| 9 | 270 | GYESDNHTA | (SEQ ID NO:105) | 72.000 |
| 10 | 326 | AYPGCNKRY | (SEQ ID NO:42) | 60.000 |
| 11 | 192 | QYSVPPPVY | (SEQ ID NO:176) | 60.000 |
| 12 | 272 | ESDNHTAPI | (SEQ ID NO:70) | 57.600 |
| 13 | 289 | HTHGVFRGI | (SEQ ID NO:113) | 57.600 |
| 14 | 126 | DVRDLNALL | (SEQ ID NO:62) | 57.600 |
| 15 | 4 | CTGSQALLL | (SEQ ID NO:52) | 57.600 |
| 16 | 208 | SCTGSQALL | (SEQ ID NO:202) | 48.000 |
| 17 | 441 | NMTKLQLAL | (SEQ ID NO:149) | 48.000 |
| 18 | 207 | DSCTGSQAL | (SEQ ID NO:61) | 48.000 |
| 19 | 130 | NAPYLPSCL | (SEQ ID NO:144) | 48.000 |
| 20 | 235 | CMTWNQMNL | (SEQ ID NO:49) | 48.000 |

TABLE 39

RESULTS OF BIMAS HLA PEPTIDE BINDING PREDICTION ANALYSIS FOR BINDING OF
HUMAN WT1 PEPTIDES TO MOUSE MHC CLASS I KK

| | | | |
|---|---|---|---|
| | | | Score (Estimate of Half Time |
| | Start | Disassociation of a Molecule | |
| Rank | Position | Subsequence Residue Listing | Containing This Subsequence) |

| | | | | |
|---|---|---|---|---|
| 1 | 81 | AEPHEEQCL | (SEQ LDNO:30) | 40.000 |
| 2 | 85 | EEQCLSAFT | (SEQ ID NO:65) | 40.000 |
| 3 | 429 | DELVRHHNM | (SEQ ID NO:53) | 20.000 |
| 4 | 315 | SETSEKRPF | (SEQ ID NO:209) | 20.000 |
| 5 | 261 | TEGQSNHST | (SEQ ID NO:221) | 20.000 |
| 6 | 410 | SEKPFSCRW | (SEQ ID NO:207) | 10.000 |
| 7 | 272 | ESDNHTTPI | (SEQ ID NO:71) | 10.000 |
| 8 | 318 | SEKRPFMCA | (SEQ ID NO:208) | 10.000 |
| 9 | 138 | LESQPAIRN | (SEQ ID NO:132) | 10.000 |
| 10 | 233 | LECMTWNQM | (SEQ ID NO:131) | 10.000 |
| 11 | 298 | QDVRRVPGV | (SEQ ID NO:164) | 10.000 |
| 12 | 84 | HEEQCLSAF | (SEQ ID NO:107) | 10.000 |
| 13 | 349 | GEKPYQCDF | (SEQ ID NO:91) | 10.000 |
| 14 | 289 | HTHGVFRGI | (SEQ ID NO:113) | 10.000 |
| 15 | 179 | EDPMGQQGS | (SEQ ID NO:64) | 8.000 |
| 16 | 136 | SCLESQPAI | (SEQ ID NO:198) | 5.000 |
| 17 | 280 | ILCGAQYRI | (SEQ ID NO:116) | 5.000 |
| 18 | 273 | SDNHTTPIL | (SEQ ID NO:204) | 4.000 |
| 19 | 428 | SDELVRHHN | (SEQ ID NO:203) | 4.000 |
| 20 | 3 | SDVRDLNAL | (SEQ ID NO:206) | 4.000 |

TABLE 40

RESULTS OF BIMAS HLA PEPTIDE BINDING PREDICTION ANALYSIS FOR BINDING OF
HUMAN WT1 PEPTIDES TO MOUSE MHC CLASS I LD

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 163 | TPSHHAAQF | (SEQ ID NO:228) | 360.000 |
| 2 | 327 | YPGCNKRYF | (SEQ ID NO:250) | 300.000 |
| 3 | 180 | DPMGQQGSL | (SEQ ID NO:59) | 150.000 |
| 4 | 26 | LPVSGAAQW | (SEQ ID NO:138) | 93.600 |
| 5 | 278 | TPILCGAQY | (SEQ ID NO:227) | 72.000 |
| 6 | 141 | QPAIRNQGY | (SEQ ID NO:170) | 60.000 |
| 7 | 219 | TPYSSDNLY | (SEQ ID NO:231) | 60.000 |
| 8 | 303 | VPGVAPTLV | (SEQ ID NO:242) | 60.000 |
| 9 | 120 | ASSGQARMF | (SEQ ID NO:40) | 50.000 |
| 10 | 63 | PPPPPPHSF | (SEQ ID NO:158) | 45.000 |
| 11 | 113 | GPPPPSQAS | (SEQ ID NO:97) | 45.000 |
| 12 | 157 | TPSYGHTPS | (SEQ ID NO:229) | 39.000 |
| 13 | 207 | DSCTGSQAL | (SEQ ID NO:61) | 32.500 |
| 14 | 110 | GPFGPPPPS | (SEQ ID NO:96) | 30.000 |
| 15 | 82 | EPHEEQCLS | (SEQ ID NO:68) | 30.000 |
| 16 | 412 | KPFSCRWPS | (SEQ ID NO:123) | 30.000 |
| 17 | 418 | WPSCQKKFA | (SEQ ID NO:246) | 30.000 |
| 18 | 221 | YSSDNLYQM | (SEQ ID NO:253) | 30.000 |
| 19 | 204 | TPTDSCTGS | (SEQ ID NO:230) | 30.000 |
| 20 | 128 | FPNAPYLPS | (SEQ ID NO:79) | 30.000 |

TABLE 41

RESULTS OF BIMAS HLA PEPTIDE BINDING PREDICTION ANALYSIS FOR BINDING OF
HUMAN WT1 PEPTIDES TO CATTLE HLA A20

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 350 | EKPYQCDFK | (SEQ ID NO:66) | 1000.00 |
| 2 | 319 | EKRPFMCAY | (SEQ ID NO:67) | 500.000 |
| 3 | 423 | KKFARSDEL | (SEQ ID NO:122) | 500.000 |
| 4 | 345 | RKHTGEKPY | (SEQ ID NO:184) | 500.000 |
| 5 | 390 | RKFSRSDHL | (SEQ ID NO:183) | 500.000 |
| 6 | 137 | CLESQPAIR | (SEQ ID NO:47) | 120.000 |
| 7 | 380 | VKPFQCKTC | (SEQ ID NO:239) | 100.000 |
| 8 | 407 | GKTSEKPFS | (SEQ ID NO:95) | 100.000 |
| 9 | 335 | FKLSHLQMH | (SEQ ID NO:78) | 100.000 |
| 10 | 247 | LKGVAAGSS | (SEQ ID NO:135) | 100.000 |
| 11 | 370 | LKRHQRRHT | (SEQ ID NO:136) | 100.000 |
| 12 | 258 | VKWTEGQSN | (SEQ ID NO:240) | 100.000 |
| 13 | 398 | LKTHTRTHT | (SEQ ID NO:137) | 100.000 |
| 14 | 331 | NKRYFKLSH | (SEQ ID NO:145) | 100.000 |
| 15 | 357 | FKDCERRFS | (SEQ ID NO:77) | 100.000 |
| 16 | 385 | CKTCQRKFS | (SEQ ID NO:46) | 100.000 |
| 17 | 294 | FRGIQDVRR | (SEQ ID NO:81) | 80.000 |
| 18 | 368 | DQLKRHQRR | (SEQ ID NO:60) | 80.000 |
| 19 | 432 | VRHHNMHQR | (SEQ ID NO:243) | 80.000 |
| 20 | 118 | SQASSGQAR | (SEQ ID NO:216) | 80.000 |

TABLE 42

RESULTS OF BIMAS HLA PEPTIDE BINDING PREDICTION ANALYSIS FOR BINDING OF
MOUSE WT1 PEPTIDES TO MOUSE MHC CLASS I A_0201

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 126 | RMFPNAPYL | (SEQ ID NO:293) | 313.968 |
| 2 | 187 | SLGEQQYSV | (SEQ ID NO:299) | 285.163 |
| 3 | 10 | ALLPAVSSL | (SEQ ID NO:255) | 181.794 |
| 4 | 225 | NLYQMTSQL | (SEQ ID NO:284) | 68.360 |
| 5 | 292 | GVFRGIQDV | (SEQ ID NO:270) | 51.790 |
| 6 | 93 | TLHFSGQFT | (SEQ ID NO:302) | 40.986 |
| 7 | 191 | QQYSVPPPV | (SEQ ID NO:290) | 22.566 |
| 8 | 280 | ILCGAQYRI | (SEQ ID NO:274) | 17.736 |
| 9 | 441 | NMTKLHVAL | (SEQ ID NO:285) | 15.428 |
| 10 | 235 | CMTWNQMNL | (SEQ ID NO:258) | 15.428 |
| 11 | 7 | DLNALLPAV | (SEQ ID NO:261) | 11.998 |
| 12 | 242 | NLGATLKGM | (SEQ ID NO:283) | 11.426 |
| 13 | 227 | YQMTSQLEC | (SEQ ID NO:307) | 8.573 |
| 14 | 239 | NQMNLGATL | (SEQ ID NO:286) | 8.014 |
| 15 | 309 | TLVRSASET | (SEQ ID NO:303) | 7.452 |
| 16 | 408 | KTSEKPFSC | (SEQ ID NO:277) | 5.743 |
| 17 | 340 | LQMHSRKHT | (SEQ ID NO:280) | 4.752 |
| 18 | 228 | QMTSQLECM | (SEQ ID NO:289) | 4.044 |
| 19 | 37 | VLDFAPPGA | (SEQ ID NO:304) | 3.378 |
| 20 | 302 | RVSGVAPTL | (SEQ ID NO:295) | 1.869 |

TABLE 43

RESULTS OF BIMAS HLA PEPTIDE BINDING PREDICTION ANALYSIS FOR BINDING OF
MOUSE WT1 PEPTIDES TO MOUSE MHC CLASS I DB

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 221 | YSSDNLYQM | (SEQ ID NO:308) | 312.000 |
| 2 | 126 | RMFPNAPYL | (SEQ ID NO:293) | 260.000 |
| 3 | 235 | CMTWNQMNL | (SEQ ID NO:258) | 260.000 |
| 4 | 437 | MHQRNMTKL | (SEQ ID NO:281) | 200.000 |
| 5 | 238 | WNQMNLGAT | (SEQ ID NO:305) | 12.000 |
| 6 | 130 | NAPYLPSCL | (SEQ ID NO:282) | 8.580 |
| 7 | 3 | SDVRDLNAL | (SEQ ID NO:298) | 7.920 |
| 8 | 136 | SCLESQPTI | (SEQ ID NO:296) | 7.920 |
| 9 | 81 | AEPHEEQCL | (SEQ ID NO:254) | 6.600 |
| 10 | 10 | ALLPAVSSL | (SEQ ID NO:255) | 6.600 |
| 11 | 218 | RTPYSSDNL | (SEQ ID NO:294) | 6.000 |
| 12 | 441 | NMTKLHVAL | (SEQ ID NO:285) | 3.432 |
| 13 | 228 | QMTSQLECM | (SEQ ID NO:289) | 3.120 |
| 14 | 174 | HSFKHEDPM | (SEQ ID NO:272) | 3.120 |
| 15 | 242 | NLGATLKGM | (SEQ ID NO:283) | 2.640 |
| 16 | 261 | TEGQSNHGI | (SEQ ID NO:301) | 2.640 |
| 17 | 225 | NLYQMTSQL | (SEQ ID NO:284) | 2.640 |
| 18 | 207 | DSCTGSQAL | (SEQ ID NO:263) | 2.600 |
| 19 | 119 | QASSGQARM | (SEQ ID NO:288) | 2.600 |
| 20 | 18 | LGGGGGCGL | (SEQ ID NO:279) | 2.600 |

TABLE 44

RESULTS OF BIMAS HLA PEPTIDE BINDING PREDICTION ANALYSIS FOR BINDING OF
MOUSE WT1 PEPTIDES TO MOUSE MHC CLASS I KB

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 329 | GCNKRYFKL | (SEQ ID NO:268) | 24.000 |
| 2 | 225 | NLYQMTSQL | (SEQ ID NO:284) | 10.000 |
| 3 | 420 | SCQKKFARS | (SEQ ID NO:297) | 3.960 |
| 4 | 218 | RTPYSSDNL | (SEQ ID NO:294) | 3.630 |
| 5 | 437 | MHQRNMTKL | (SEQ ID NO:281) | 3.600 |
| 6 | 387 | TCQRKFSRS | (SEQ ID NO:300) | 3.600 |
| 7 | 289 | HTHGVFRGI | (SEQ ID NO:273) | 3.000 |
| 8 | 130 | NAPYLPSCL | (SEQ ID NO:282) | 3.000 |
| 9 | 43 | PGASAYGSL | (SEQ ID NO:287) | 2.400 |
| 10 | 155 | DGAPSYGHT | (SEQ ID NO:260) | 2.400 |
| 11 | 126 | RMFPNAPYL | (SEQ ID NO:293) | 2.200 |
| 12 | 128 | FPNAPYLPS | (SEQ ID NO:267) | 2.000 |
| 13 | 207 | DSCTGSQAL | (SEQ ID NO:263) | 1.584 |
| 14 | 3 | SDVRDLNAL | (SEQ ID NO:298) | 1.584 |
| 15 | 332 | KRYFKLSHL | (SEQ ID NO:276) | 1.500 |
| 16 | 233 | LECMTWNQM | (SEQ ID NO:278) | 1.320 |
| 17 | 18 | LGGGGGCGL | (SEQ ID NO:279) | 1.320 |
| 18 | 242 | NLGATLKGM | (SEQ ID NO:283) | 1.200 |
| 19 | 123 | GQARMFPN | (SEQ ID NO:269)A | 1.200 |
| 20 | 441 | NMTKLHVAL | (SEQ ID NO:285) | 1.200 |

TABLE 45

RESULTS OF BIMAS HLA PEPTIDE BINDING PREDICTION ANALYSIS FOR BINDING OF
MOUSE WT1 PEPTIDES TO MOUSE MHC CLASS I KD

| | | | |
|---|---|---|---|
| | | | Score (Estimate of Half Time of |
| | Start | | Disassociation of a Molecule |
| Rank | Position | Subsequence Residue Listing | Containing This Subsequence) |

| | | | | |
|---|---|---|---|---|
| 1 | 285 | QYRIHTHGV | (SEQ ID NO:291) | 600.000 |
| 2 | 424 | KFARSDELV | (SEQ ID NO:275) | 288.000 |
| 3 | 334 | YFKLSHLQM | (SEQ ID NO:306) | 120.000 |
| 4 | 136 | SCLESQPTI | (SEQ ID NO:296) | 115.200 |
| 5 | 239 | NQMNLGATL | (SEQ ID NO:286) | 115.200 |
| 6 | 10 | ALLPAVSSL | (SEQ ID NO:255) | 115.200 |
| 7 | 47 | AYGSLGGPA | (SEQ ID NO:256) | 86.400 |
| 8 | 180 | DPMGQQGSL | (SEQ ID NO:262) | 80.000 |
| 9 | 270 | GYESDNHTA | (SEQ ID NO:271) | 72.000 |
| 10 | 192 | QYSVPPPVY | (SEQ ID NO:292) | 60.000 |
| 11 | 326 | AYPGCNKRY | (SEQ ID NO:257) | 60.000 |
| 12 | 289 | HTHGVFRGI | (SEQ ID NO:273) | 57.600 |
| 13 | 4 | DVRDLNALL | (SEQ ID NO:264) | 57.600 |
| 14 | 126 | RMFPNAPYL | (SEQ ID NO:293) | 57.600 |
| 15 | 209 | CTGSQALLL | (SEQ ID NO:259) | 48.000 |
| 16 | 86 | EQCLSAFTL | (SEQ ID NO:265) | 48.000 |
| 17 | 302 | RVSGVAPTL | (SEQ ID NO:295) | 48.000 |
| 18 | 218 | RTPYSSDNL | (SEQ ID NO:294) | 48.000 |
| 19 | 272 | ESDNHTAPI | (SEQ ID NO:266) | 48.000 |
| 20 | 225 | NLYQMTSQL | (SEQ ID NO:284) | 48.000 |

TABLE 46
RESULTS OF TSITES PEPTIDE BINDING PREDICTION ANA-
LYSIS FOR HUMAN WT1 PEPTIDES CAPABLE OF ELICITING
A HELPER T CELL RESPONSE

| Peptide | Sequence | |
|---|---|---|
| p6-23 | RDLNALLPAVPSLGGGG | (SEQ ID NO:1) |
| p30-35 | GAAQWA | (SEQ ID NO:309) |
| p45-56 | ASAYGSLGGPAP | (SEQ ID NO:310) |
| p91-105 | AFTVHFSGQFTGTAG | (SEQ ID NO:311) |
| p117-139 | PSQASSGQARMFPNAPYLPSCLE | (SEQ ID NO:2) |
| p167-171 | HAAQF | (SEQ ID NO:312) |
| p202-233 | CHTPTDSCTGSQALLLRTPYSSDNLYQ | (SEQ ID NO:313) |
| | MTSQL | |
| p244-262 | GATLKGVAAGSSSSVKWTE | (SEQ ID NO:4) |
| p287-318 | RIHTHGVFRGIQDVRRVPGVAPTLVRS | (SEQ ID NO:314) |
| | ASETS | |
| p333-336 | RYFK | (SEQ ID NO:315) |
| p361-374 | ERRFSRSDQLKRHQ | (SEQ ID NO:316) |
| p389-410 | QRKFSRSDHLKTHTRTHTGKTS | (SEQ ID NO:317) |
| p421-441 | CQKKFARSDELVRHHNMHQRN | (SEQ ID NO:318) |

Certain CTL peptides (shown in Table 47) were selected for further study. For each peptide in Table 47, scores obtained using BIMAS HLA peptide binding prediction analysis are provided.

TABLE 47

WT1 PEPTIDE SEQUENCES AND HLA PEPTIDE BINDING PREDICTIONS

| | | |
|---|---|---|
| Peptide | Sequence | Comments |

| | | | |
|---|---|---|---|
| p329-337 | GCNKRYFKL | (SEQ ID NO:90 and SEQ ID NO:268) | Score 24,000 |
| p225-233 | NLYQMTSQL | (SEQ ID NO:147 and SEQ ID NO:284) | binds also to class II |
| | | | and HLA A2, Kd, score |
| | | | 10,000 |
| p235-243 | CMTWNQMNL | (SEQ ID NO:49 and SEQ ID NO:258) | binds also to HLA A2, |
| | | | score 5,255,712 |
| p126-134 | RMFPNAPYL | (SEQ ID NO:185 and SEQ ID NO:293) | binds also to Kd, class |
| | | | II and HLA A2, score |
| | | | 1,990,800 |
| p221-229 | YSSDNLYQM | (SEQ ID NO:253 and SEQ ID NO:308) | binds also to Ld, score |
| | | | 312,000 |
| p228-236 | QMTSQLECM | (SEQ ID NO:169 and SEQ ID NO:289) | score 3,120 |
| p239-247 | NQMNLGATL | (SEQ ID NO:151 and SEQ ID NO:286) | binds also to HLA A |
| | | | 0201, Kd, score 8,015 |
| mouse p136-144 SCLESQPTI | (SEQ ID NO:296) | binds also to Kd, | |
| | | | 1mismatch to human |
| human p136-144 SCLESQPAI | (SEQ ID NO:198) | score 7,920 | |
| mouse p10-18 | ALLPAVSSL | (SEQ ID NO:255) | binds also to Kd, HLA |
| | | | A2, 1 mismatch to |
| | | | human |
| human p10-18 | ALLPAVPSL | (SEQ ID NO:34) | score 6,600 |

Peptide binding to C57Bl/6 murine MHC was confirmed using the leukemia cell line RMA-S, (Ljunggren et al., 1990). In brief, RMA-S cells were cultured for 7 hrs at 26° C. in complete medium supplemented with 1% FCS. A total of 106 RMA-S cells were added into each well of a 24-well plate and incubated either alone or with the designated peptide (25 μg/ml) for 16 hrs at 26° C. and additional 3 hrs at 37° C. in complete medium. Cells were then washed three times and stained with fluorescein isothiocyanate-conjugated anti Db or anti-Kb antibody (PharMingen, San Diego, CA). Labeled cells were washed twice, resuspended and fixed in 500 μl of PBS with 1% paraformaldehyde and analyzed for fluorescence intensity in a flow cytometer (Becton-Dickinson FACSCalibur®). The percentage of increase of D^{b} or K^{b} molecules on the surface of the RMA-S cells was measured by increased mean fluorescent intensity of cells incubated with peptide compared with that of cells incubated in medium alone.

Mice were immunized with the peptides capable of binding to murine class I MHC. Following immunization, spleen cells were stimulated in vitro and tested for the ability to lyse targets incubated with WT1 peptides. CTL were evaluated with a standard chromium release assay (Chen et al., 1994). 106 target cells were incubated at 37° C. with 150 μCi of sodium ⁵¹Cr for 90 min., in the presence or absence of specific peptides. Cells were washed three times and resuspended in RPMI with 5% fetal bovine serum. For the assay, 10^{4 51}Cr-labeled target cells were incubated with different concentrations of effector cells in a final volume of 200 μl in U-bottomed 96-well plates. Supernatants were removed after 4-7 hrs at 37° C., and the percentage specific lysis was determined by the formula:

% Specific lysis=100×(experimental release−spontaneous release)/(maximum release-spontaneous release).

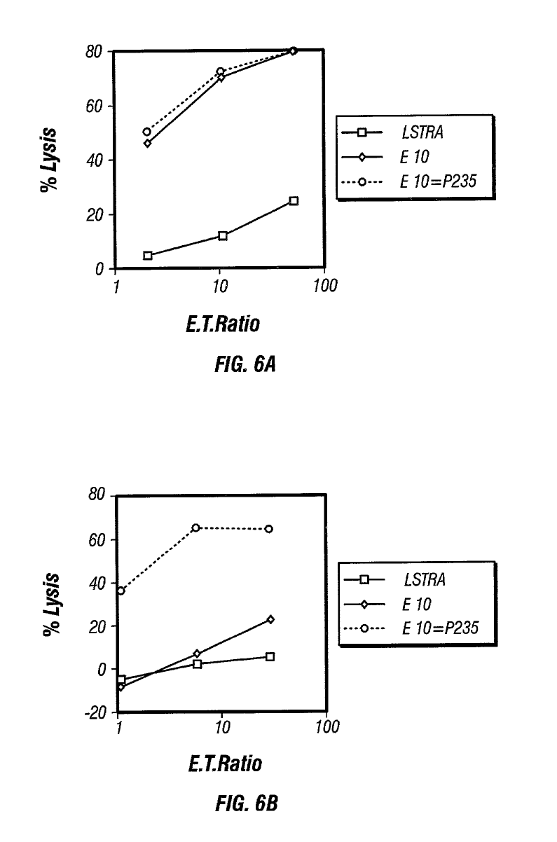

The results, presented in Table 48, show that some WT1 peptides can bind to class I MHC molecules, which is essential for generating CTL. Moreover, several of the peptides were able to elicit peptide specific CTL (FIG. 6A and FIG. 6B), as determined using chromium release assays. Following immunization to CTL peptides p10-18 human, p136-144 human, p136-144 mouse and p235-243, peptide specific CTL lines were generated and clones were established. These results indicate that peptide specific CTL can kill malignant cells expressing WT1.

TABLE 48
BINDING OF WT1 CTL PEPTIDES TO MOUSE B6
CLASS I ANTIGENS

| | Binding Affinity to Mouse MHC |
|---|---|
| Peptide | Class I |
| Positive control | 91% |
| Negative control | 0.5.-1.3% |
| p235-243 | 33.6% |
| p136-144 mouse | 27.9% |
| p136-144 human | 52% |
| p10-18 human | 2.2% |
| p225-233 | 5.8% |
| p329-337 | 1.2% |
| p126-134 | 0.9% |
| p221-229 | 0.8% |
| p228-236 | 1.2% |
| p239-247 | 1% |

### 5.5 Example 5

### Use of a WT1 Peptide to Elicit WT1 Specific CTL in Mice

This example illustrates the ability of a representative WT1 peptide to elicit CTL immunity capable of killing WT1 positive tumor cell lines.

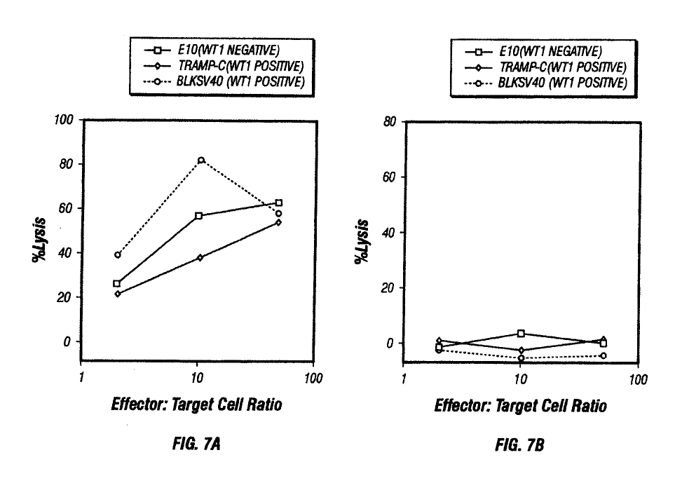

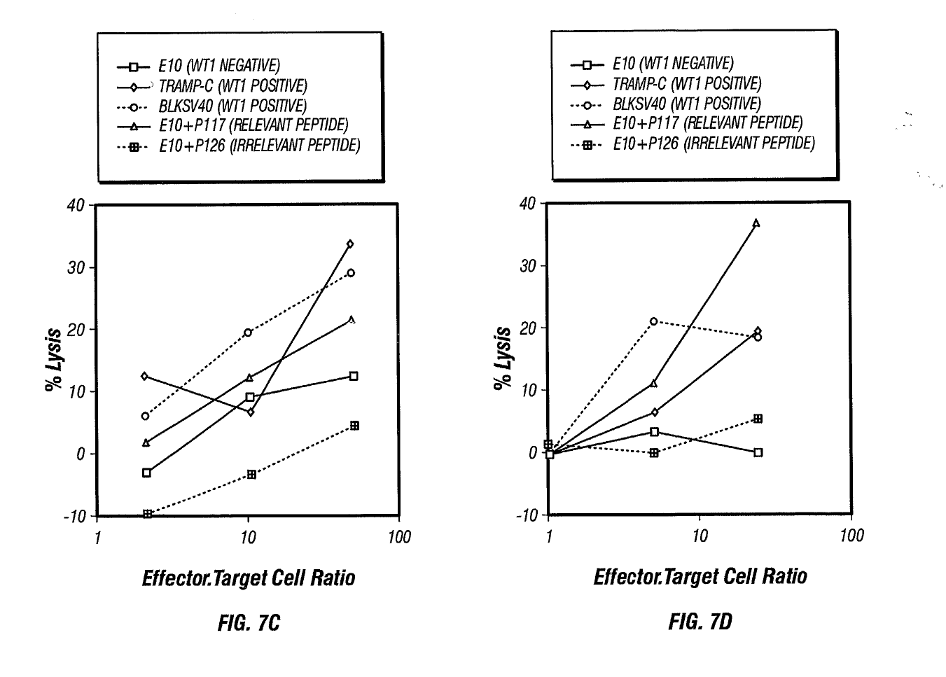

P117-139, a peptide with motifs appropriate for binding to class I and class II MHC, was identified as described above using TSITES and BIMAS HLA peptide binding prediction analyses. Mice were immunized as described in Example 3. Following immunization, spleen cells were stimulated in vitro and tested for the ability to lyse targets incubated with WT1 peptides, as well as WT1 positive and negative tumor cells. CTL were evaluated with a standard chromium release assay. The results, presented in FIG. 7A, FIG. 7B, FIG. 7C, and FIG. 7D, show that P117 can elicit WT1 specific CTL capable of killing WT1 positive tumor cells, whereas no killing of WT1 negative cells was observed. These results demonstrate that peptide specific CTL in fact kill malignant cells expressing WT1 and that vaccine and T cell therapy are effective against malignancies that express WT1.

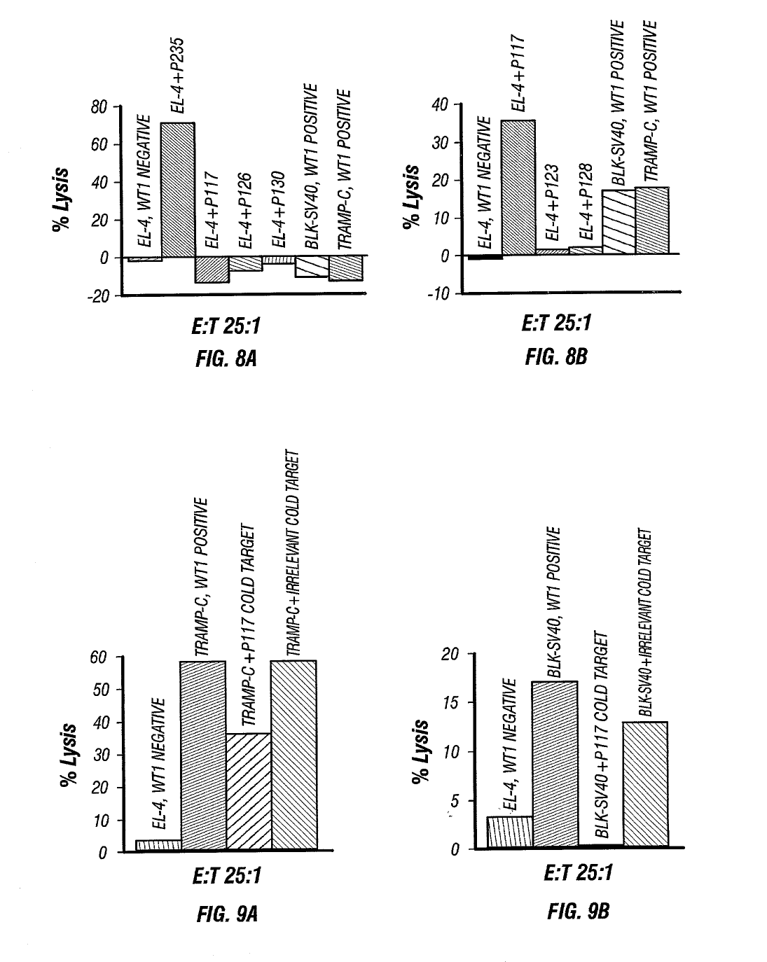

Similar immunizations were performed using the 9-mer class-I MHC binding peptides p136-144, p225-233, p235-243 as well as the 23-mer peptide, p117-139. Following immunization, spleen cells were stimulated in vitro with each of the 4 peptides and tested for ability to lyse targets incubated with WT1 peptides. CTL were generated specific for p136-144, p235-243 and p117-139, but not for p225-233. CTL data for p235-243 and pl 17-139 are presented in FIG. 8A and FIG. 8B. Data for peptides p136-144 and p225-233 are not depicted.

CTL lysis demands that the target WT1 peptides are endogenously processed and presented in association with tumor cell class I MHC molecules. The above WT1 peptide specific CTL were tested for ability to lyse WT1 positive versus negative tumor cell lines.

CTL specific for p235-243 lysed targets incubated with the p235-243 peptides, but failed to lyse cell lines that expressed WT1 proteins (FIG. 8A). By marked contrast, CTL specific for pll7-139 lysed targets incubated with pll7-139 peptides and also lysed malignant cells expressing WT1 (FIG. 8B). As a negative control, CTL specific for pl 17-139 did not lyse WT1 negative EL-4 (also referred to herein as E10).

Specificity of WT1 specific lysis was confirmed by cold target inhibition (FIG. 9A and FIG. 9B). Effector cells were plated for various effector:target ratios in 96-well U-bottom plates. A ten-fold excess (compared to hot target) of the indicated peptide-coated target without ⁵Cr labeling was added. Finally, 10^{4 51}Cr-labeled target cells per well were added and the plates incubated at 37° C. for 4 hrs. The total volume per well was 200 μl.

Lysis of TRAMP-C by pl 17-139 specific CTL was blocked from 58% to 36% by EL-4 incubated with the relevant peptide pl 17-139, but not with EL-4 incubated with an irrelevant peptide (FIG. 9A). Similarly, lysis of BLK-SV40 was blocked from 18% to 0% by EL-4 incubated with the relevant peptide pl 17-139 (FIG. 9B). Results validate that WT1, peptide specific CTL specifically kill malignant cells by recognition of processed WT1.

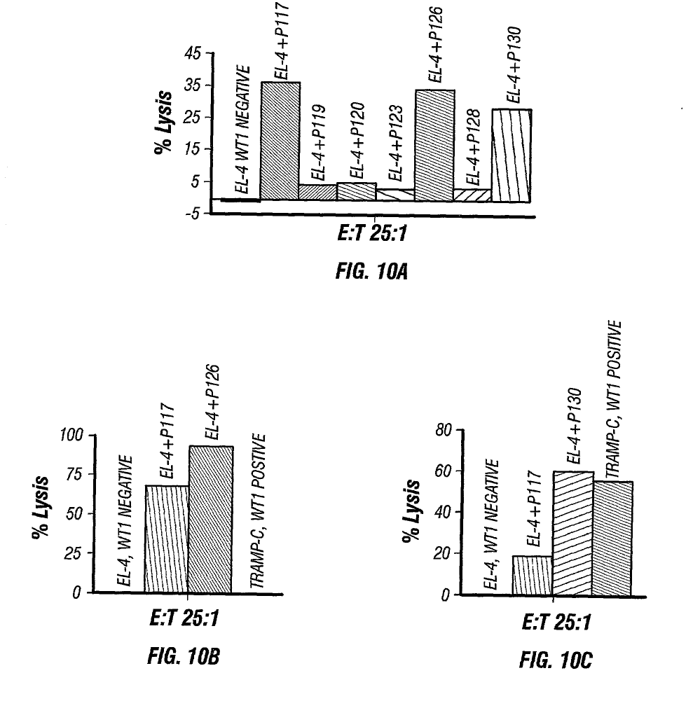

Several segments with putative CTL motifs are contained within pl 17-139. To determine the precise sequence of the CTL epitope all potential 9-mer peptides within p117-139 were synthesized (Table 49). Two of these peptides (p126-134 and p130-138) were shown to bind to H-2b class I molecules (Table 49). CTL generated by immunization with p117-139 lysed targets incubated with p126-134 and p130-138, but not the other 9-mer peptides within p117-139 (FIG. 10A).

The p117-139 specific CTL line was restimulated with either p126-134 or p130-138. Following restimulation with p126-134 or p130-138, both T cell lines demonstrated peptide specific lysis, but only p130-138 specific CTL showed lysis of a WT1 positive tumor cell line (FIG. 10B and FIG. 10C). Thus, p130-138 appears to be the naturally processed epitope.

TABLE 49

BINDING OF WT1 CTL 9-MER PEPTIDES WITHIN p117-139 TO MOUSE B6 CLASS
I ANTIGENS

| | |
|---|---|
| | Binding Affinity |
| | to Mouse MHC |
| Peptide | Class I |

| | | | |
|---|---|---|---|
| P117-125 | PSQASSGQA | (SEQ ID NO:221) | 2% |
| P118-126 | SQASSGQAR | (SEQ ID NO:216) | 2% |
| P119-127 | QASSGQARM | (SEQ ID NO:161 and SEQ ID NO:288) | 2% |
| P120-128 | ASSGQARMF | (SEQ ID NO:40 | 1% |
| P121-129 | SSGQARMFP | (SEQ ID NO:222) | 1% |
| P122-130 | SGQARMFPN | (SEQ ID NO:212) | 1% |
| P123-131 | GQARMFPNA | (SEQ ID NO:98 and SEQ ID NO:269) | 1% |
| P124-132 | QARMFPNAP | (SEQ ID NO:223) | 1% |
| P125-133 | ARMFPNAPY | (SEQ ID NO:38) | 1% |
| P126-134 | RMFPNAPYL | (SEQ ID NO:185 and SEQ ID NO:293) | 79% |
| P127-135 | MFPNAPYLP | (SEQ ID NO:224) | 2% |
| P128-136 | FPNAPYLPS | (SEQ ID NO:79 and SEQ ID NO:267) | 1% |
| P129-137 | PNAPYLPSC | (SEQ ID NO:225) | 1% |
| P130-138 | NAPYLPSCL | (SEQ ID NO:144 and SEQ ID NO:282) | 79% |
| P131-139 | APYLPSCLE | (SEQ ID NO:226) | 1% |

### 5.6 Example 6

### Identification of WT1 Specific mRNA in Mouse Tumor Cell Lines

This example illustrates the use of RT-PCR™to detect WT1 specific mRNA in cells and cell lines.

Mononuclear cells were isolated by density gradient centrifugation, and were immediately frozen and stored at −80° C. until analyzed by RT-PCR™ for the presence of WT1 specific mRNA. RT-PCR™ was generally performed as described by Fraizer et al. (1995). Total RNA was extracted from 10⁷ cells according to standard procedures. RNA pellets were resuspended in 25 μL diethylpyrocarbonate treated water and used directly for reverse transcription. The zinc-finger region (exons 7 to 10) was amplified by PCR™ as a 330-bp mouse cDNA. Amplification was performed in a thermocycler during one or, when necessary, two sequential rounds of PCR™. AmpliTaq DNA Polymerase (Perkin Elmer Cetus, Norwalk, Conn.), 2.5 mM MgCl₂ and 20 pmol of each primer in a total reaction volume of 50 μl were used. Twenty μL aliquots of the PCR™ products were electrophoresed on 2% agarose gels stained with ethidium bromide. The gels were photographed with Polaroid film (Polaroid 667, Polaroid Ltd., Hertfordshire, England). Precautions against cross contamination were taken following the recommendations of Kwok and Higuchi (1989). Negative controls included the cDNA- and PCR™-reagent mixes with water instead of cDNA in each experiment. To avoid false negatives, the presence of intact RNA and adequate cDNA generation was evaluated for each sample by a control PCR™ using P-actin primers. Samples that did not amplify with these primers were excluded from analysis.

Primers for amplification of WT1 in mouse cell lines were: P115: 1458-1478: 5′-CCCAGGCTGCAATAAGAGATA-3′ (forward primer; SEQ ID NO:21); and P116: 1767-1787: 5′-ATGTTGTGATGGCGGACCAAT-3′ (reverse primer; SEQ ID NO:22) (Inoue et al., 1996;Fraizeretal., 1995).

Beta Actin primers used in the control reactions were: 5′-GTGGGGCGCCCCAGGCACCA-3′ (sense primer; SEQ ID NO:23); and 5′-GTCCTTAATGTCACGCACGATTTC-3′ (antisense primer; SEQ ID NO:24).

Primers for use in amplifying human WT1 include: P117: 954-974: 5′-GGCATCTGAGACCAGTGAGAA-3′ (SEQ ID NO:25); and P118: 1434-1414: 5′-GAGAGTCAGACTTGAAAGCAGT-3′ (SEQ ID NO:5). For nested RT-PCR M, primers may be: P119: 1023-1043: 5′-GCTGTCCCACTTACAGATGCA-3′ (SEQ ID NO:26); and P120: 1345-1365: 5′-TCAAAGCGCCAGCTGGAGTTT-3′ (SEQ ID NO:27).

Table 50 shows the results of WT1 PCR™ analysis of mouse tumor cell lines. Within Table 5, (+++) indicates a strong WT1 PCR™ amplification product in the first step RT-PCR™, (++) indicates a WT1 amplification product that is detectable by first step WT1, RT-PCR™, (+) indicates a product that is detectable only in the second step of WT1 RT-PCR™, and (−) indicates WT1 PCR™ negative.

TABLE 50
DETECTION OF WT1 MRNA IN MOUSE TUMOR CELL LINES

| Cell Line | WT1 mRNA |
|---|---|
| K562 (human leukemia; ATCC): | +++ |
| Positive control (Lozzio and Lozzio, 1975) | |
| TRAMPC (SV4O transformed prostate, B6) | +++ |
| (Foster et al., 1997) | |
| BLK-SV40 HD2 (SV40-transf. fibroblast, B6; ATCC) | ++ |
| (Patek et al., 1978) | |
| CTLL (T-cell, B6; ATCC) (Gillis, 1977) | + |
| FM (FBL-3 subline, leukemia, B6) (Glynn et al., 1968) | + |
| BALB 3T3 (ATCC) (Aaroston and Todaro, 1968) | + |
| S49.1 (Lymphoma, T-cell like, B/C; ATCC) | + |
| (Horibata and Harris, 1970) | |
| BNL CL.2 (embryonic liver, B/C; ATCC) | + |
| (Patek et al., 1978) | |
| MethA (sarcoma, B/C) (Old et al., 1962) | − |
| P3.6.2.8.1 (myeloma, B/C; ATCC) (Watson et al., 1970) | − |
| P2N (leukemia, DBA/2; ATCC) (Melling et al., 1976) | − |
| BCL1 (lymphoma, B/C; ATCC) (Slavin and Strober, 1977) | − |
| LSTRA (lymphoma, B/C) (Glynn et al., 1968) | − |
| E10/EL-4 (lymphoma, B6) (Glynn et aL, 1968) | − |

### 5.7 Example 7

### Evaluation of the Systemic Histopathological and Toxicological Effects of WT1 Immunization in Mice

The purpose of this example is to analyze the immunogenicity and potential systemic histopathological and toxicological effects of WT1 protein immunization in a multiple dose titration in mice.

The experimental design for immunization of mice with WT1 protein is outlined in Table 51.

TABLE 51
EXPERIMENTAL DESIGN OF WT1 IMMUNIZATION IN MICE

| Histology | Corixa | | Dose | Total No. |
|---|---|---|---|---|
| Group | Group | Treatment Description | Level | (Females) |
| 1 | 0 | No treatment | 0 | 4 |
| 2 | 1.1 | MPL-SE (adjuvants alone), 6×, | 10 μg | 4 |
| | | 1 week apart | | |
| 3 | 1.2 | MPL-SE, 3×, 2 weeks apart | 10 μg | 4 |
| 4 | 2.1 | Ra12-WT1 + MPL-SE, 6× | 25 μg | 4 |
| 5 | 2.2 | Ra12-WT1 + MPL-SE, 3× | 25 μg | 4 |
| 6 | 3.1 | Ra12-WT1 + MPL-SE, 6× | 100 μg | 4 |
| 7 | 3.2 | Ra12-WT1 + MPL-SE, 3× | 100 μg | 4 |
| 8 | 4.1 | Ra12-WT1 + MPL-SE, 6× | 1000 μg | 4 |
| 9 | 4.2 | Ra12-WT1 + MPL-SE, 3× | 1000 μg | 4 |

Vaccination to WT1 protein using MPL-SE as adjuvant, in a multiple dose titration study (doses ranging from 25 μg, 100 μg to 1000μg WT1 protein) in female C57/B6 mice elicited a strong WT1-specific antibody response (FIG. 19) and cellular T-cell responses (FIG. 20).

No systemic histopathological or toxicological effects of immunization with WT1 protein were observed. No histological evidence for toxicity was seen in the following tissues: adrenal gland, brain, cecum, colon, duodenum, eye, femur and marrow, gall bladder, heart, ileum, jejunum, kidney, larynx, lacrimal gland, liver, lung, lymph node, muscle, esophagus, ovary, pancreas, parathyroid, salivary gland, sternum and marrow, spleen, stomach, thymus, trachea, thyroid, urinary bladder and uterus.

Special emphasis was put on evaluation of potential hematopoietic toxicity. The myeloid/erythroid ratio in sternum and femur marrow was normal. All evaluable blood cell counts and blood chemistry (BUN, creatinine, bilirubin, albumin, globulin) were within the normal range.

Given that existent immunity to WT1 is present in some patients with leukemia and that vaccination to WT1 protein can elicit WT1-specific Ab and cellular T-cell responses in mice without toxicity to normal tissues, these experiments validate WT1 as a tumor/leukemia vaccine.

### 5.8 EXAMPLE 8

### Elicitation of Human WT1-specific T-Cell Responses by Whole Gene In vitro Priming

This example demonstrates that WT1 specific T-cell responses can be generated from the blood of normal individuals.

Dendritic cells (DC) were differentiated from monocyte cultures derived from PBMC of normal donors by growth for 4-10 days in RPMJ medium containing 10% human serum, 50 ng/ml GMCSF and 30 ng/ml IL-4. Following culture, DC were infected 16 hrs with recombinant WT1-expressing vaccinia virus at an M.O.I. of 5, or for 3 days with recombinant WT1-expressing adenovirus at an M.O.I. of 10. Vaccinia virus was inactivated by U.V. irradiation. CD8⁺ T-cells were isolated by positive selection using magnetic beads, and priming cultures were initiated in 96-well plates. Cultures were restimulated every 7-10 days using autologous dendritic cells adeno or vaccinia infected to express WT1. Following 3-6 stimulation cycles, CD8+lines could be identified that specifically produced interferon-gamma when stimulated with autologous-WT1-expressing dendritic cells or fibroblasts. The WT1-specific activity of these lines could be maintained following additional stimulation cycles. These lines were demonstrated to specifically recognize adeno or vaccinia WT1 infected autologous dendritic cells but not adeno or vaccinia EGFP-infected autologous dendritic cells by Elispot assays.

### 5.9 Example 9

### Formulation of Ra12-WT1 for Injection: Use of Excipients to Stabilize Lyophilized Product

This example describes the formulation that allows the complete solubilization of lyophilized Ra12-WT1.

The following formulation allowed for the recombinant protein Ra12-WT1 to be dissolved into an aqueous medium after being lyophylized to dryness:

Recombinant Ra12-WT1 concentration: 0.5 -1.0 mg/ml; Buffer: 10-20 mM Ethanolamine, pH 10.0; 1.0-5.0 mM Cysteine; 0.05 % Tween-80 (Polysorbate-80); Sugar: 10% Trehalose (T5251, Sigma, MO) 10% Maltose (M9171, Sigma, MO) 10% Sucrose (S7903, Sigma, MO) 10% Fructose (F2543, Sigma, MO) 10% Glucose (G7528, Sigma, MO).

The lyophilized protein with the sugar excipient was found to dissolve significantly more than without the sugar excipient. Analysis by Coomassie stained SDS-PAGE showed no signs of remaining solids in the dissolved material.

### 5.10 Example 10

### Identification of an Immune Response to WT1 in Patients with Hematological Malignancies

This Example illustrates the identification of an existent immune response in patients with a hematological malignancy.

To evaluate the presence of preexisting WT1 specific antibody responses in patients, sera of patients with acute myelogenous leukemia (AML), acute lymphocytic leukemia (ALL), chronic myelogenous leukemia (CML) and severe aplastic anemia were analyzed using Western blot analysis. Sera were tested for the ability to immunoprecipitate WT1 from the human leukemic cell line K562 (American Type Culture Collection, Manassas, Va.). In each case, immunoprecipitates were separated by gel electrophoresis, transferred to membrane and probed with the anti WT-1 antibody WT180 (Santa Cruz Biotechnology, Inc., Santa Cruz, Calif.). This Western blot analysis identified potential WT1 specific antibodies in patients with hematological malignancy. A 52-kDa protein in the immunoprecipitate generated using the patient sera was recognized by the WT1 specific antibody. The 52-kDa protein migrated at the same size as the positive control.

Additional studies analyzed the sera of patients with AML and CML for the presence of antibodies to full-length and truncated WT1 proteins. CDNA constructs representing the human WT1/full-length (aa 1-449), the N-terminus (aa 1-249) (WT1/N-terminus) and C-terminus (aa 267-449) (WT1/C-terminus) region were subcloned into modified pET28 vectors. The WT1/full-length and WT1/IN-terminus proteins were expressed as Ra12 fusion proteins. Ra12 is the C-terminal fragment of a secreted Mycobacterium tuberculosis protein, denoted as MTB32B. (Skeiky et al., 1999). The Ra12-WT1/full-length fusion region was cloned 3′ to a histidine-tag in a histidine-tag modified pET28 vector. The WT1/N-terminus region was subcloned into a modified pET28 vector that has a 5′ histidine-tag followed by the thioredoxin (TRX)-WT1/N-terminus fusion region followed by a 3′-histidine-tag. The WT1/C-terminus coding region was subcloned into a modified pET28 vector without a fusion partner containing only the 5′ and 3′ histidine-tag, followed by a Thrombin and EK site.

BL21 pLysS E. coli (Stratagene, La Jolla, Calif.) were transformed with the three WT1 expression constructs, grown overnight and induced with isopropyl-P-D-thiogalactoside (IPTG). WT1 proteins were purified as follows: Cells were harvested and lysed by incubation in 10 mM Tris, pH 8.0 with Complete Protease Inhibitor Tablets (Boehringer Mannheim Biochemicals, Indianapolis, Ind.) at 37° C. followed by repeated rounds of sonication. Inclusion bodies were washed twice with 10 mM Tris, pH 8.0. Proteins were then purified by metal chelate affinity chromatography over nickel-nitrilotriacetic acid resin (QIAGEN Inc., Valencia, Calif.) followed by chromatography on a Source Q anion exchange resin (Amersham Pharmacia Biotech, Upsala, Sweden). The identity of the WT1 proteins was confirmed by N-terminal sequencing.

Sera from adult patients with de nova AML or CML were studied for the presence of WT1 specific Ab. Recombinant proteins were adsorbed to TC microwell plates (Nunc, Roskilde, Denmark). Plates were washed with PBS/0.5% Tween-20 and blocked with 1% BSA/PBS/0.1% Tween-20. After washing, serum dilutions were added and incubated overnight at 4° C. Plates were washed and Donkey anti-human IgG-HRP secondary antibody was added (Jackson-Immunochem, West Grove, Pa.) and incubated for 2 hrs at room temperature. Plates were washed, incubated with TMB Peroxidase substrate solution (Kirkegaard and Perry Laboratories, Mass.), quenched with 1 N H₂SO₄, and immediately read (Cyto-Fluor 2350; Millipore, Bedford, Mass.).

For the serological survey, human sera were tested by ELISA over a range of serial dilutions from 1:50 to 1:20,000. A positive reaction was defined as an OD value of a 1:500 diluted serum that exceeded the mean OD value of sera from normal donors (n=96) by three (WT1,/full-length, WT1C-terminus) standard deviations. Due to a higher background in normal donors to the WT1/N-terminus Protein A positive reaction to WT1/N-terminus was defined as an OD value of 1:500 diluted serum that exceeded the mean OD value of sera from normal donors by four standard deviations. To verify that the patient Ab response was directed against WT1 and not to the Ra12 or TRX fusion part of the protein or possible E. coli contaminant proteins, controls included the Ra12 and TRX Protein Alone purified in a similar manner. Samples that showed reactivity against the Ra12 and/or TRX proteins were excluded from the analysis.

To evaluate for the presence of immunity to WT1, Ab to recombinant full-length and truncated WT1 proteins in the sera of normal individuals and patients with leukemia were determined. Antibody reactivity was analyzed by ELISA reactivity to WT1/full-length protein, WT1/N-terminus Protein And WT1/C-terminus protein.

Only 2 of 96 normal donors had serum antibodies reactive with WT1,/full-length protein. One of those individuals had antibody to WT1/N-terminus Protein And one had antibody to WT1/C-terminus protein. In contrast, 16 of 63 patients (25%) with AML had serum antibodies reactive with WT1/full-length protein. By marked contrast, only 2 of 63 patients (3%) had reactivity to WT1/C-terminus protein. Fifteen of 81 patients (19%) with CML had serum antibodies reactive with WT1/full-length Protein And 12 of 81 patients (15%) had serum antibodies reactive with WT1I/N-terminus. Only 3 of 81 patients (3%) had reactivity to WT1/C-terminus protein.

These data demonstrated that Ab responses to WT1 are detectable in some patients with AML and CML. The greater incidence of antibody in leukemia patients provides strong evidence that immunization to the WT1 protein occurred as a result of patients bearing malignancy that expresses or at some time expressed WT1,. Without being limited to a specific theory, it is believed that the observed antibody responses to WT1 most probably result from patients becoming immune to WT1 on their own leukemia cells and provide direct evidence that WT1 can be immunogenic despite being a “self” protein.

The presence of antibody to WT1 strongly implies that concurrent helper T cell responses are also present in the same patients. WT1 is an internal protein. Thus, CTL responses are likely to be the most effective in terms of leukemia therapy and the most toxic arm of immunity. Thus, these data provide evidence that therapeutic vaccines directed against WT1 will be able to elicit an immune response to WT1.

The majority of the antibodies detected were reactive with epitopes within the N-terminus while only a small subgroup of patients showed a weak antibody response to the C-terminus. This is consistent with observations in the animal model, where immunization with peptides derived from the N-terminus elicited antibody, helper T cell and CTL responses, whereas none of the peptides tested from the C-terminus elicited antibody or T cell responses (Gaiger et al., 2000).

### 6. REFERENCES

The following references, to the extent that they provide exemplary procedural or other details supplementary to those set forth herein, are specifically incorporated herein by reference.

U.S. Pat. No. 3,817,827.

U.S. Pat. No. 3,850,752.

U.S. Pat. No. 3,901,654.

U.S. Pat. No. 3,935,074.

U.S. Pat. No. 3,984,533.

U.S. Pat. No. 3,996,345.

U.S. Pat. No. 4,034,074.

U.S. Pat. No. 4,098,876.

U.S. Pat. No. 4,235,877.

U.S. Pat. No. 4,376,110.

U.S. Pat. No. 4,429,008.

U.S. Pat. No. 4,436,727.

U.S. Pat. No. 4,452,901.

U.S. Pat. No. 4,489,710.

U.S. Pat. No. 4,507,234.

U.S. Pat. No. 4,554,101.

U.S. Pat. No. 4,569,789.

U.S. Pat. No. 4,603,112.

U.S. Pat. No. 4,625,014.

U.S. Pat. No. 4,638,045.

U.S. Pat. No. 4,671,958.

U.S. Pat. No. 4,673,562.

U.S. Pat. No. 4,683,195.

U.S. Pat. No. 4,699,784.

U.S. Pat. No. 4,735,792.

U.S. Pat. No. 4,751,180.

U.S. Pat. No. 4,769,330.

U.S. Pat. No. 4,777,127.

U.S. Pat. No. 4,866,034.

U.S. Pat. No. 4,873,088.

U.S. Pat. No. 4,877,611.

U.S. Pat. No. 4,897,268.

U.S. Pat. No. 4,912,094.

U.S. Pat. No. 4,935,233.

U.S. Pat. No. 5,017,487.

U.S. Pat. No. 5,075,109.

U.S. Pat. No. 5,145,684.

U.S. Pat. No. 5,151,254.

U.S. Pat. No. 5,215,926.

U.S. Pat. No. 5,240,856.

U.S. Pat. No. 5,350,840.

U.S. Pat. No. 5,359,681.

U.S. Pat. No. 5,399,346.

U.S. Pat. No. 5,399,363.

U.S. Pat. No. 5,466,468.

U.S. Pat. No. 5,472,869.

U.S. Pat. No. 5,543,158.

U.S. Pat. No. 5,552,157.

U.S. Pat. No. 5,565,213.

U.S. Pat. No. 5,567,434.

U.S. Pat. No. 5,633,234.

U.S. Pat. No. 5,641,515.

U.S. Pat. No. 5,738,868.

U.S. Pat. No. 5,741,516.

U.S. Pat. No. 5,795,587.

U.S. Pat. No. 5,811,128.

U.S. Pat. No. 5,814,344.

U.S. Pat. No. 5,820,883.

U.S. Pat. No. 5,853,763.

U.S. Pat. No. 5,874,265.

U.S. Pat. No. 5,928,647.

U.S. Pat. No. 5,942,252.

U.S. Pat. No. 6,110,702.

European Patent No. EP 0,345,242.

Great Britain Patent No. GB 2,200,651.

Intl. Pat. App′. Publ. No. WO 89/01973.

Intl. Pat. Appl. Publ. No. WO 89/06280.

Intl. Pat. Appl. Publ. No. WO 91/02805.

Intl. Pat. App,. Pubi. No. WO 91/16116.

Intl. Pat. Appl. Publ. No. WO 92/07245.

Intl. Pat. Appl. Publ. No. WO 94/00153.

Intl. Pat. Appl. Publ. No. WO 94/20078.

Intl. Pat. Appl. Publ. No. WO/94/23701.

Intl. Pat. Appl. Publ. No. WO 95/17210.

Intl. Pat. Appi. Pubi. No. WO 96/02555.

Intl. Pat. Appl. Publ. No. WO 96/06638.

Intl. Pat. Appl. Publ. No. WO 96/30516.

Intl. Pat. Appl. Publ. No. WO 96/33739.

Intl. Pat. Appl. Publ. No. WO 97/24447.

Intl. Pat. Appl. Publ. No. WO 99/33488.

Aaroston and Todaro, J. Cell. Physiol., 72:141-48, 1968.

Adelman et al., DNA, 2:183, 1983.

Aminetal., Am. J Pathol., 146:344-56, 1995.

Akaza et al., “Expression of antitumor response. Role of attachment and viability of bacillus Calmette-Guerin to bladder cancer cells,” Cancer, 72:558-63, 1993.

American Type Culture Collection, Catalogue of Cell Lines and Hybridomas, 7th ed., 1992.

Avrameas, “Natural autoantibodies: Self-recognition and physiological autoimmunity,” In: Natural autoantibodies: Their Physiological Role and Regulatory Significance, Shoenfeld and Isenberg (eds.), CRC Press, Boca Raton, Fla., pp. 1-14, 1993.

Azuma et al., “Correlation Between Augmented Resistance to Influenza Virus Infection and Histological Changes in Lung of Mice Treated with Trehalose-6,6′-dimycolate,” Journal of Biological Response Modifiers, 7:473-82, 1988.

Bajorin et al., Proc. Annu. Meet. Am. Soc. Clin. Oncol., 7:A967, 1988.

Baker et al., “Ability of Monophosphoryl Lipid A To Augment the Antibody Response of Young Mice,” Infection and Immunity, 56:3064-66, 1988a.

Baker et al., “Enrichment of Suppressor T Cells by Means of Binding to Monophosphoryl Lipid A,” Infection and Immunity, 58:726-31, 1990.

Baker et al., “Inactivation of Suppressor T-Cell Activity by Nontoxic Monophosphoryl Lipid A,” Infection and Immunity, 56:1076-83, 1988b.

Baker et al., “Molecular structures that influence the immunomodulatory properties of the lipid A and inner core region oligosaccharides of bacterial lipopolysaccharides,” Infection Immunity, 62:2257-69, 1994.

Baker et al., “Structural Features That Influence the Ability of Lipid A and Its Analogs To Abolish Expression of Suppressor T Cell Activity,” Infection and Immunity, 60:2694-701, 1992.

Bakker et al., “Melanocyte lineage-specific antigen gplOO is recognized by melanoma-derived tumor-infiltrating lymphocytes,” J Exp. Med., 179:1005, 1994.

Banchereau and Steinman, Nature, 392:245-51, 1998.

Banerji et al., “Membrane lipid composition modulates the binding specificity of a monoclonal antibody against liposomes,” Biochim. Biophys. Acta., 689:319-26, 1982.

Barnd et al., “Specific tumor histocompatibility complex-unrestricted recognition of tumor-associated mucins by human cytotoxic T cells,” Proc. Natl. Acad. Sci. USA, 86:7159, 1989.

Bamoud et al., Am. J. Surg. Pathol., 24:830-36, 2000).

Bartlett and Zbar, J Natl. Cancer Inst., 48:1709, 1972.

Bast et al., “BCG and Cancer,” N. Engl. J Med., 290:1413-20, 1974.

Bennett et al., “Endogenous Production of Cytotoxic Factors in Serum of BCG-Primed Mice by Monophosphoryl Lipid A, a Detoxified Form of Endotoxin,” Journal of Biological Response Modifiers, 7:65-76, 1988

Berkner, Biotechniques, 6:616-27, 1988.

Berra et al., Int. J Cancer, 36:363-66, 1985.

Berra et al., J Neurochem., 40:777-82, 1983.

Bogoch, “Demonstration of serum precipitin to brain gangliosides,” Nature, 183:392-93, 1960.

Bouchon et al., Biochem. Internatl., 10:531-38, 1985.

Bowen-Pope et al., Proc. Nat'l Acad. Sci. USA, 81:2396-400, 1984.

Bowness et al, “Clostridium perfringens enterotoxin is a superantigen reactive with human T cell receptors V beta 6.9 and V beta 22,” J. Exp. Med., 176:893-96, 1992.

Brade et al., “An Artificial Glycoconjugate Containing the Bisphosphorylated Glucosamine Disaccharide Backbone of Lipid A Binds Lipid A Monoclonal Antibodies,” Infection and Immunity, 61:4514-17, 1993.

Brichard et al., “The tyrosinase gene codes for an antigen recognized by autologous cytolytic T lymphocytes on HLA-A2 melanomas,” J. Exp. Med., 178:489, 1993.

Brodin et al., “Mouse monoclonal antibodies with specificity for the melanoma-associated ganglioside disialyllactosyl ceramide (GD3) also react with the structural analogue disialylparagloboside,” Biochim. Biophys. Acta., 837:349-53, 1985.

Brooks et al., Clin. Exp. Immunol., 39: 477, 1980.

Brown et al., J Biol. Chem., 255:4980-83, 1980.

Burchell et al., J Immunol., 131:508-13, 1983.

Bystryn et al., Cancer, 61:1065, 1988.

Cahan et al., “Identification of a human neuroectodermal tumor antigen (OFA-I-2) as ganglioside GD2,” Proc. Natl. Acad. Sci. USA, 79:7629-33, 1982.

Campbell et al., “Intercellular adhesion molecule-1 expression by bladder cancer cells: functional effects,” J Urol., 151:1385-90, 1994.

Campbell, in Monoclonal Antibody Technology, Laboratory Techniques in Biochemistry and Molecular Biology, Vol. 13, Burden and Von Knippenberg (eds.), Amsterdam, Elseview, pp. 75-83, 1984.

Campbell et al. Int. J. Cancer, 78:182-88, 1998.

Carr and Morrison, “A two-step mechanism for the interaction of Re lipopolysaccharide with erythrocyte membranes,” Rev. Infect. Dis. 6:497-508, 1984.

Carubia et al., Biochem. Biophys. Res. Commun., 120:500-04, 1984.

Chang et al., Crit. Rev. Oncol. Hematol., 22:213, 1996.

Chase et al., “Effect of Monophosphoryl Lipid A on Host Resistance to Bacterial Infection,” Infection and Immunity, 53(3):711-12, 1986.

Chen et al., “Activation of Macrophages From Aging Mice by Detoxified Lipid A,” Journal of Leukocyte Biology, 49:416-22, 1991.

Chen et al., Cancer Res., 54:1065-70, 1994.

Cheng et al., “Bacillus Calmette-Gerin interacts with the carboxyl-terminal heparin bindings domain of fibronectin: implications for BCG-mediated antitumor activity,” J Urol., 152:1275-80, 1994.

Cheresh and Klier, “Disialoganglioside GD3 distributes preferentially into substrate associated microprocesses on human melanoma cells during their attachment to fibronectin,” J. Cell. Biol., 102:1887-97, 1986.

Cheresh et al., “A monoclonal antibody recognizes an 0-acetyl sialic acid in a human melanoma-associated ganglioside,” J. Biol. Chem., 259:7453-59, 1984.

Cheresh et al., “Disialoganglioside GD3 on human melanoma serves as a relevant target antigen for monoclonal antibody-mediated tumor cytolysis,” Proc. Natl. Acad. Sci. USA, 82:5155-59, 1985.

Cheresh et al., “Disialogangliosides GD2 and GD3 are involved in the attachment of human melanoma and neuroblastoma cells to extracellular matrix proteins,” J Cell. Biol., 102:688-96,1986.

Cheresh et al., “Localization of gangliosides GD2 and GD3 in adhesion plaques and on the surface of human melanoma cells,” Proc. Natl. Acad Sci. USA, 81:5767-71, 1984.

Cheung et al., “Detection of neuroblastoma cells in bone marrow using GD2 specific monoclonal antibodies,” J Clin. Oncol., 4:363-69, 1986.

Chu and Sharom, “Gangliosides inhibit T-lymphocyte proliferation by preventing the interaction of interleukin-2 with its cell surface receptors,” Immunology, 79:10-16, 1993.

Cohen, Science, 259:1691-92, 1993.

Colcher et al., Proc. Natl. Acad. Sci. USA, 78:3199, 1987.

Coligan et al., in Current Protocols in Immunology, Vol 1, Wiley Interscience. Greene (ed.), 1998.

Coombes et al., Vaccine, 14:1429-38, 1996.

Coulie et al., “A new gene coding for a differentiation antigen recognized by autologous cytologic T lymphocytes on HLA-A2 melanomas,” J Exp. Med., 180:35, 1994.

Deavin et al., Mol. Immunol., 33:145-55, 1996.

DeBruijn et al., Eur. J. Immunol., 21:2963-70, 1991.

Dippold et al., “Immunohistochemical localization of ganglioside GD3 in human malignant melanoma, epithelial tumors and normal tissues,” Cancer Res., 45:3699-705, 1985.

Dippold et al., “Inflammatory response at the tumor site after systemic application of monoclonal anti-GD3-ganglioside antibody to patients with malignant melanoma,” Am. Assoc. Cancer Res., 978:247, 1984.

Dippold et al., Proc. Natl. Acad. Sci. USA, 77:6115, 1980.

Dresser and Phillips, in Immunopotentiation, CIBA Foundation Symposium 18, Elsevier, Amsterdam, p.3, 1973.

Dwivedi et al., “Plasma lipid-bound sialic acid alterations in neoplastic diseases,” Experientia, 46:91-94, 1990.

Elder, “Skin Cancer,” Cancer, 75:245-56, 1995.

Elliott et al., “The D-Galactosamine Loaded Mouse and Its Enhanced Sensitivity to Lipopolysaccharide and Monophosphoryl Lipid A: A Role for Superoxide,” J Immunol., 10:69-74, 1991.

Euhus et al., Cancer Immunol Immunother., 29:247-54, 1989.

Fawwaz et al., Statutory Invention Registration Patent No. H819, application no. 6-6-5,439, 1990.

Fischer, Handb. Lipid Res., 6:123-234, 1990.

Fisher-Hoch et al., Proc. Nat'l Acad. Sci. USA, 86:317-21, 1989.

Fitzgerald, “Syphilis vaccine: up-regulation of immunogenicity by cyclophosphamide, Ribi adjuvant, and indomethacin confers significant protection against challenge infection in rabbits,” Vaccine, 9:265-72, 1991.

Fleischmann, Park and Hassan, “Fibronectin expression on surgical specimens correlated with the response to intravesical bacillus Calmette-Guerin therapy,” J. Urol., 149:268-71, 1993.

Flexner etal., Ann. N.Y. Acad. Sci., 569:86-103, 1989.

Flexner et al., Vaccine, 8:17-21, 1990.

Foster et al., Cancer Res., 57:3325-30, 1997.

Fraizer et al., Blood, 86:4704-06, 1995.

Fredman et al., Neurol. Res., 8:123-26, 1986.

Freimer et al., “Gangliosides elicit a T-cell independent antibody response,” J. Autoimmun., 6:281-89, 1993.

Freudenberg et al., “ELISA for antibodies to Lipid A, Lipopolyasscharides and other hydrophobic antigens,” Infection, 17:322-24, 1989.

Gaiger et al., Blood, 96:1334, 2000

Garg and Subbarao, “Immune Responses of Systemic and Mucosal Lymphoid Organs to Pnu-Immune Vaccine as a Function of Age and the Efficacy of Monophosphoryl Lipid A as an Adjuvant,” Infection and Immunity, 60:2329-36, 1992.

Gaugler et al., “Human gene MAGE-3 codes for an antigen recognized on a human melanoma by autologous cytolytic T lymphocytes,” J. Exp. Med., 179:921, 1994.

Gefter et al., Somatic Cell Genet., 3:231-36, 1977.

Gennaro et al., Am. J. Ind. Med., 37:275-82, 2000.

Gillard et al, “Antibodies against ganglioside GT₃ in the sera of patients with type I Diabetes mellitus,” J Immunol., 142:3826-32, 1989.

Gillis, Nature, 268:154-56, 1977.

Glynn, McCoy and Fefer, Cancer Res., 28:434-39, 1968.

Goding, in Monoclonal Antibodies: Principles and Practice, 2d ed., Orlando, Fla., Academic Press, pp. 60-61, 65-66, 71-74, 1986.

Goff et al., Eur. J. Biochem., 130:553-57, 1983.

Grabarek et al., “Endotoxic Lipid A Interaction with Human Platelets,” The Journal of Biological Chemistry, 265:8117-21, 1990.

Graus et al., “Distribution of the ganglioside GD3 in the human nervous system detected by R24 mouse monoclonal antibody,” Brain Res., 324:190-94, 1984.

Guzman et al., Circulation, 88:2838-48, 1993a.

Guzman et al, Cir. Res., 73:1202-07, 1993b.

Hachida et al, Transplant Proc., 22:1663-70, 1990.

Hachida et al, Transplantation, 56:479-82, 1993.

Harada et al., Mol. Urol, 3:357-364, 1999.

Hardings et al, “Effects of pH and polysaccharides on peptide binding to class II major histocompatibility complex molecules,” Proc. Natl. Acad. Sci. USA, 88:2740-44, 1991.

Harel et al., Cancer Res., 50:6311, 1990.

Harlow and Lane, Antibodies: A Laboratory Manual, Cold Spring Harbor Laboratory, 1988.

Helling et al., “Construction of Immunogenic GD₃-conjugate vaccines,” Ann. N.Y. Acad. Sci., 690:396-97, 1993.

Hellstrom et al, “Strong anti-tumor activities of IgG 3 antibodies to a human melanoma-associated ganglioside,” Proc. Natl. Acad. Sci. USA, 82:1499-1502, 1985.

Hirabayashi et al., “Syngeneic monoclonal antibody against melanoma antigen with interspecies cross-reactivity recognize GM₃, a prominent ganglioside of B 16 melanoma,” Biol. Chem., 260:13328-33, 1985.

Hirabayashi et al., “Reactivity of mouse monoclonal antibody M2590 against B 16 melanoma cells with chemically synthesized GM3 ganglioside,” Biochim. Biophys. Acta, 875:126-28, 1986.

Hirabayashi et al, Jpn. J Cancer Res., 78:614-20, 1987.

Hoon et al., “Gangliosides from melanoma immunomodulate response of T-cells to interleukin-2,” Cell Immunol., 4:1111 - 19, 1988.

Horibata and Harris, Exp. Cell Res., 60:61, 1970.

Horgan, “Total and lipid-bound sialic acid levels in sera from patients with Cancer,” Clin. Chim. Acta., 118:327-31, 1982.

Houghten et al., “Mouse monoclonal IgG3 antibody detecting GD3 ganglioside: A phase I trial in patients with malignant melanoma,” Proc. Natl. Acad. Sci. USA, 82:1242-46, 1985.

Houghton, “Cancer Antigens: Immune Recognition of Self and Altered Self,” J. Exp. Med., 180:1-4, 1994.

Hraba et al., “The Influence of Monophosphoryl Lipid A (MPL™) on Erythrocyte Autoantibody Formation,” Immunobiol., 189:448-56, 1993.

Hunter et al., Vaccine, 9:250: 1991.

Inoue et al., Blood, 88:2267-78, 1996.

Irie and Morton, “Regression of cutaneous metastatic melanoma by intralesional injection with human monoclonal antibody to ganglioside GD2,” Proc. Natl. Acad. Sci. USA, 83:8694-98, 1986.

Irie and Ravindranath, “Gangliosides as targets for monoclonal antibody therapy of cancer,” in Therapeutic monoclonal antibodies, Borrebaeck and Larrick (eds.), Stockton Press, New York, p. 75-94, 1990.

Irie et al., “Human antibody to OFA-1, a tumor antigen, produced in vitro by Epstein-Barr virus transformed human B-lymphoid cell lines,” Proc. Nati. Acad. Sci. USA, 79:5666-70, 1982.

Irie et al., “Melanoma gangliosides and human monoclonal antibody,” in Human Tumor Antigens and Specific Tumor Therapy, Metzgar and Mitchell (eds.), Alan R. Liss, Inc., New York, pp. 115-126, 1989.

Ishioka et al., “MHC interaction and T cell recognition of carbohydrates and glycopeptides,” J Immunol., 148:2446-51, 1992.

Jackson et al., “Induction of ICAM 1 expression on bladder tumours by BCG immunotherapy,” J Clin. Pathol, 47:309-12, 1994.

Johnson and Tomai, “A Study of the Cellular and Molecular Mediators of the Adjuvant Action of a Nontoxic Monophosphoryl Lipid A,” Adv. Exp. Med. Biol., 133:567-79, 1988.

Johnson et al., “Characterization of a nontoxic monophosphoryl lipid A,” Rev. Infect. Dis., 9:512-16, 1987.

Johnson et al., “Structural Characterization of Monophosphoryl Lipid A Homologs Obtained from Salmonella minnesota Re595 Lipopolysaccharide,” J. Biol. Chem., 265:8108-16, 1990.

Johnston and Bystryn, “Effect of Cell Wall Skeleton and Monophosphoryl Lipid A Adjuvant on the Immunogenicity of a Murine B16 Melanoma Vaccine,” Journal of the National Cancer Institute, 83:1240-45, 1991.

Jones et al., J. Natl Cancer Inst, 66:249-54, 1981.

Kabat et al., “Sequences of Proteins of Immunological Interest,” 5th ed., Public Health Service, National Institutes of Health, Bethesda, Md., pp 647-669, 1991.

Kass-Eisler et al., Proc. Nat'l Acad. Sci. USA, 90:11498-502, 1993.

Katopodis et al., “Lipid-associated sialic acid test for the detection of human cancer,” Cancer Res., 42:5270-75, 1982.

Kawaguchi et al., “Characteristic mode of action of gangliosides in selective modulation of CD4 on human T lymphocytes,” Biochem. Biophys. Res. Commun., 158:1050-55, 1989.

Kawakami et al., “Cloning of the gene coding for a shared human melanoma antigen recognized by autologous T cells infiltrating into tumor,” Proc. Nat/. Acad. Sci. USA, 91:3515, 1994.

Kawakami, Eliyahu, Sakaguchi, Robbins, Rivoltini, Yannelli, Appella and Rosenberg, “Identification of the immunodominant peptides of the Mart-1 human melanoma antigen recognized by the majority of HLA-A2 restricted tumor infiltrating lymphocytes,” J Exp. Med., 180:347-52, 1994.

Kensil etal., J Am. Vet. Med. Assoc., 199:1423, 1991.

Kloppel et al., “Glycolipid-bound sialic acid in serum: Increased levels in mice and humans bearing mammary carcinomas,” Proc. NatL. Acad. Sci. USA, 74:3011-13, 1977.

Kohler and Milstein, Nature, 256:495-97, 1975.

Kohler and Milstein, Eur. J Immunol., 6:511-19, 1976.

Kolls et al., Proc. Nat'l Acad. Sci. USA, 91:215 -19, 1994.

Koscielak et al., “Glycolipid antigen and its antibody,” Immunochemistry, 5:441-55, 1968.

Kovach et al., “Lipid IV_{A} Inhibits Synthesis and Release of Tumor Necrosis Factor Induced by Lipopolysaccharide in Human Whole Blood Ex Vivo,” J Exp. Med., 172:77-84, 1990.

Kwok and Higuchi, Nature, 339:237-38, 1989.

Kyogashima et al., Jpn. J. Cancer Res., 78:1229-32, 1987.

Ladisch et al., “Shedding of GD2 ganglioside by human neuroblastoma,” Int. J. Cancer, 39:73-76, 1987.

Lamm et al., “A randomized trial of intravesical doxorubicin and immunotherapy with bacille Calmette-Guerin for transitional-cell carcinoma of the bladder,” N. Engl. J. Med., 325:1205, 1991.

Lengle etal., “Inhibition of the lectin-induced mitogenic response of thymocytes by glycolipids,” Cancer Res., 39:817-922, 1979.

Liepkalns et al., J. Neurochem., 36:1959-65, 1981.

Livingston et al., “The Serologic Response to Meth A Sarcoma Vaccines After Cyclophosphamide Treatment is Additionally Increased by Various Adjuvants,” The Journal ofImmunology, 135(2):1505-09, 1985.

Livingston et al., “Approaches to augmenting immunogenicity of the ganglioside GM₂ in mice: purified GM₂ is superior to whole cells,” J Immunol., 138:1524-29, 1987a.

Livingston et al., “Vaccines containing purified GM₂ gangliosides elicit GM₂ antibodies in melanoma patients,” Proc. Natl. Acad. Sci. USA, 84:2911-15, 1987b.

Ljunggren et al., Nature, 346:476-80, 1990.

Lozzio and Lozzio, Blood, 45:321-34, 1975.

Madonna and Vogel, “Induction of Early-Phase Endotoxin Tolerance in Athymic (Nude) Mice, B-Cell-Deficient (xid) Mice, and Splenectomized Mice,” Infection and Immunity, 53:707-10, 1986.

Mahvi et al., Immunology and cell Biology, 75:456-60, 1997.

Maratea et al., Gene,40:39-46, 1985.

Masihi et al., “Effects of Nontoxic Lipid A and Endotoxin on Resistance of Mice to Toxoplasma gondii,” Journal of Biological Response Modifiers, 7:535-39, 1988

Melling et al., J Immunol., 117:1267-74, 1976.

Menssen et al., J. Cancer Res. Clin. Oncol., 126:226-32, 2000.

Merrifield, J Am. Chem. Soc., 85:2149-46, 1963.

Miller and Esselman, “Modulation of immune response by antigen reactive lymphocytes after cultivation with gangliosides,” J Immunol., 115:839-43, 1975.

Minden, “Shared Antigens Between Animal and Human Tumors and Microorganisms,” in BCG in Cancer Immunotherapy, Lamoureux, Turcotte and Portelance (eds.); pp. 73-81, 1976.

Miotti et al., Cancer Res., 65:826, 1985.

Mitchell et al., “Active specific Immunotherapy of melanoma with allogeneic cell lysates: Rationale, results and possible mechanisms of action,” Ann. N.Y. Acad. Sci., 690:153-66, 1993.

Mitchell etal., “Active specific immunotherapy of melanoma: Phase I trial of allogeneic lysates and a novel adjuvant,” Cancer Res., 48:5883-93, 1988.

Mitchell et al., “Active-Specific Immunotherapy for Melanoma,” Journal of Clinical Oncology, 8:856-59, 1990.

Miyake et al., Cancer Res., 48:6154-60, 1988.

Mooney et al., “Bacterial superantigen signaling via HLA class II on human B lymphocytes,” Mol. Immunol., 31:675-81, 1994.

Morrison et al., “Specific ganglioside binding to receptor sites on T lymphocytes that couple to ganglioside-induced decrease of CD4 expression,” Life Sci., 45:1219-24, 1989.

Morton and Ravindranath, in Cancer Medicine, 3rd ed., Holland et al (eds.), Lea and Febiger, Philadelphia, p.967, 1993.

Morton et al., “Polyvalent Melanoma Vaccine Improves Survival of Patients with Metastatic Melanoma,” John Wayne Cancer Institute at Saint John's Hospital and Health Center, Santa Monica, Calif., reprinted from Specific Immunotherapy of Cancer with Vaccines, Volume 690 of the Annals of the New York Academy of Sciences, 1993.

Morton et al., Ann. Surg., 216:463, 1992.

Morton et al., in Biological Function of Gangliosides, Progress in Brain Research, Vol. 101, pp 251-275, 1994.

Mosmann and Coffman, Ann. Rev. Immunol., 7:145-73, 1989.

Munjal et al., “Combined measurement and significance of lipid-bound sialic acid and carcinoembryonic antigen in detection of human cancer,” Diagn. Immunol., 2:36-43, 1984.

Murphy et al., Proc. Natl. Acad. Sci. USA,83:8258-62, 1986.

Myers et al., “Monophosphoryl Lipid A Behaves as a T-Cell-Independent Type 1 Carrier for Hapten-Specific Antibody Responses in Mice,” Infection and Immunity, 63:168-74, 1995.

Naiki et al., “Properties of antisera to ganglioside GM₁ and AsialoGM₁”, J. Immunol., 113:84-93, 1974.

Nair et al., Nature Biotechnol., 16:364-69, 1998.

Natoli et al, “A murine monoclonal antibody detecting the ganglioside GM2: Characterization of cell surface reactivity,” Cancer Res., 46:4116-20, 1986.

Nonomura et al., Hinyokika Kiyo, 45:593-97, 1999.

Nudelman et al., “Characterization of a human melanoma-associated ganglioside antigen defined by a monoclonal antibody 4.2,” J. Biol. Chem., 257:12752-56, 1982.

Odean et al., “Involvement of Gamma Interferon in Antibody Enhancement by Adjuvants,” Infection and Immunity, 58:427-32, 1990.

Oji et al., Jpn. J. Cancer Res., 90:194-204, 1999.

Old etal., Ann. N.Y. Acad. Sci., 101:80-106, 1962.

Pan et al., Leukemia, 14:1634, 2000.

Parker et al., J. Immunol., 152:163, 1994.

Pascal et al., “Immunochemical studies on normal and Tay-Sachs′ brain gangliosides,” Proc. Soc. Exp. Biol. Med., 121:739-43, 1966.

Patek, Collins and Cohn, “Transformed cell lines susceptible or resistant to in vivo surveillance against tumorigenesis,” Nature, 276:510-11, 1978.

Patmasiriwat et al., Leukemia, 13:891-900, 1999.

Paul, Fundamental Immunology, 3rd ed., Raven Press, pp. 243-47, 1993.

Portoukalian et al., “Humoral immune response in disease-free advanced melanoma patients after vaccination with melanoma-associated gangliosides,” Int. J Cancer, 49:893-99, 1991.

Portoukalian, “Alteration of gangliosides in plasma and red cells of human bearing melanoma tumors,” Biochem. Biophys. Res. Commun., 85:916-20, 1978.

Portoukalian, “Immunoregulatory activity of gangliosides shed by melanoma tumors,” in Gangliosides and Cancer, Oettgen (ed.), New York, VCH Publishers, pp. 207-16, 1989.

Powell and Newman (eds.), “Vaccine Design (the subunit and adjuvant approach),” Plenum Press, N.Y., 1995.

Prokazova et al., “Sialylated lactosylceramides. Possible inducers of non-specific immunosuppression and atherosclerotic lesions,” Eur.J. Biochem., 171:1-10, 1988.

Pukel et al., “GD3, a prominent ganglioside of human melanoma: Detection and characterization of mouse monoclonal antibody,” J Exp. Med., 155:1133-47, 1982.

Qureshi et al., “Purification and structural determination of nontoxic lipid A obtained from the Lipopolysaccharide of Salmonella typhimurium,” J Biol. Chem., 257:11808-15, 1985.

Rabinovich et al., “Vaccine Technologies: View to the Future,” Science, 265:1401-02, 1994.

Raines and Ross, J Biol. Chem., 257:5154-60, 1982.

Rapport and Graf, “Immunochemical Reactions of Lipids,” Prog. Allergy, 13:273-331, 1969.

Ravindranath and Irie, in Malignant Melanoma: Biology, Diagnosis, and Therapy, Nathanson (ed.), Kluwer Acad., Boston, p. 17, 1988.

Ravindranath and Morton, “Role of gangliosides in active immunotherapy with melanoma vaccine,” Int. Rev. Immunol., 7:303, 1991.

Ravindranath et al., “Human melanoma antigen O-acetylated Ganglioside GD₃ is recognized by Cancer antennarius lectin,” J Biol. Chem., 263:2079-86, 1988.

Ravindranath et al., “An epitope common to gangliosides O-acetyl GD3 and GD3 recognized by antibodies in melanoma patients after active specific immunotherapy,” Cancer Res., 49:3891-97, 1989.

Ravindranath et al., “Ganglioside GM₃:GD₃ Ratio as an Index for the Management of Melanoma,” Cancer, 67:3029-35, 1991.

Ravindranath et al., “Efficacy of tumor cell vaccine after incorporating monophosphoryl lipid A (MPL) in tumor cell membranes containing tumor-associated ganglioside,” Experientia, 50:648-653, 1994a.

Ravindranath et al., “Attachment of Monophosphoryl Lipid A (MPL) to Cells and Liposomes Augments Antibody Response to membrane-bound Gangliosides,” Journal ofAutoimmunity, 7:803-16,1994b.

Ravindranath et al., “Factors affecting the fine specificity and sensitivity of serum antiganglioside antibodies in ELISA,” J Immunol. Methods, 169:257-72, 1994c.

Real et al., “Class I (unique) tumor antigens of human melanoma. Identification of a 90,000 dalton cell surface glycoprotein by autologous antibody,” J Exp. Med., 160:1219, 1984.

Reeves et al., Cancer Res., 56:5672-77, 1996.

Reisfeld et al., Melanoma Antigens and Antibodies, p. 317, 1982.

Ribi, “Beneficial modification of the endotoxin molecule,” J. Biol. Resp. Mod., 3:1-9, 1984.

Ribi et al., “Lipid A and immunotherapy,” Rev. Infect. Dis., 6:567-72, 1984.

Ribi et al., “Modulation of humoral and cell mediated immune responses by a structurally established nontoxic lipid A,” in Immunobiology and Immunopharmacology of Bacterial Endotoxins, Szentivanji and Friedman (eds.), Plenum Press, New York, pp. 407-420, 1986.

Rickman et al., Lancet 337:998, 1991.

Rolland, Crit. Rev. Therap. Drug Carrier Systems, 15:143-98, 1998.

Rosenberg et al., Ann. Surg., 210:474, 1989.

Rosenberg et al., N. Engl. J Med., 319:1676, 1988.

Rosenfeld et al., Science, 252:431-34, 1991.

Rothbard and Taylor, EMBO J, 7:93-100, 1988.

Rott et al., “Protection from experimental allergic encephalomyelitis by application of a bacterial superantigen,” Int. Immunol., 4:347-53, 1992.

Sato et al., “Cytoplasmic membrane-associated protein (CAP) isolated from Streptococcus pyrogenes: as a new bacterial superantigen,” Microbiol. Immunol., 38:139-47, 1994.

Sato et al., Science, 273:352, 1996.

Satoh et al., Pathol Int., 50:458-71, 2000.

Schuster et al., “Production of antibodies against phosphocholine, phosphatidylcholine, sphingomyelin, and lipid A by injection of liposomes containing lipid A,” J. Immunol., 122:900-05, 1979.

Schwab et al., “Superantigen can reactivate bacterial cell wall-induced arthritis,” J. Immunol., 150:4151-59, 1993.

Shafer and Spitznagel, “Sensitivity of Salmonella typhimurium to polymorphonuclear granulocyte extracts: Role of lipid A,” Rev. Infect. Dis., 6:577-81, 1984.

Shepard et al., J Clin. Immunol., 11:117-27, 1991.

Sherwin etal., “The production of antisera to gangliosides from human nervous tissue,” Canad. J Biochem., 42:1640-48, 1964.

Shimizu et al., Int. J Gynecol. Pathol., 19:158-63, 2000.

Shy et al., “Antibodies to GM₁ and GD_{1b} in patients with motor neuron disease with plasma cell dyscrasia,” Ann. Neurol., 25:511-18, 1989.

Siddiqui et al., Cancer Res., 44:5262-65, 1984.

Sidell et al., Cancer Immunol Immunother, 7:151-55, 1979.

Sigma Cell Culture, Volume 9, Number 2, 1993.

Skeiky et al., Infection and Immun., 67:3998-4007, 1999.

Slavin and Strober, Nature, 272:624-26, 1977.

Spinsanti et al., Leuk. Lymphoma, 38:611-19, 2000.

Stiess and Kruger, “Mammalian Cell Culture Media—Overview and Applications,” The Source (Sigma Cell Culture Technical and Product News), 9:1-8, 1993.

Stoute et al. New Engl. J. Med., 336:86-91, 1997.

Svennerholm et al., “Tumor gangliosides as targets for active specific immunotherapy of melanoma in man,” in Biological Function of Gangliosides, Progress in Brain Research, Vol. 101, 1994.

Tai et al., “Ganglioside GM2 as a human tumor antigen (OFA-I-1),” Proc. Natl. Acad. Sci., 80:5392-96, 1983.

Takada et al., “Molecular and Structural Requirements of a Lipoteichoic Acid from Enterococcus hirae ATCC 9790 for Cytokine-Inducing, Antitumor, and Antigenic Activities,” Infection and Immunity, 63:57-65, 1995.

Takahashi et al., J. Immunol., 140:3244, 1988.

Tamaki et al., Leukemia, 13:393-99, 1999.

Tamauchi et al., Immunology, 50:605, 1983.

Tanamoto, “Dissociation of Endotoxic Activities in a Chemically Synthesized Lipid A Precursor after Acetylation,” Infection and Immunity, 63:690-92, 1995.

Tanamoto, “Free Hydroxyl Groups Are Not Required for Endotoxic Activity of Lipid A,” Infection and Immunity, 62:1705-09, 1994a.

Tanamoto, FEBS Lett., 351:325-29, 1994b.

Tautu etal., “Improved procedure for determination of serum lipid-associated sialic acid:

Application for early diagnosis of colorectal cancer,” J. Natl. Cancer Inst., 80:1333-37, 1988.

Thor et al, Cancer Res., 46:3118, 1986.

Thurin et al., “Proton NMR and fast-atom bombardment mass spectrometry analysis of the melanoma-associated ganglioside 9-0-acetyl GD3,” J. Biol. Chem., 260:14556-563, 1985.

Timmerman and Levy, Ann. Rev. Med, 50:507-29, 1999.

Tomai and Johnson, “T Cell and Interferon-y Involvement in the Adjuvant Action of a Detoxified Endotoxin,” Journal ofBiological Response Modifiers, 8:625-43, 1989.

Tomai et al., “The Adjuvant Properties of a Nontoxic Monophosphoryl Lipid A in Hyporesponsive and Aging Mice,” Journal of Biological Response Modifiers, 6:99-107, 1987.

Tsuchida et al., J Dermatol., 11:129-38, 1984.

Tsuchida et al., “Gangliosides of Human Melanoma: Altered Expression in Vivo and in Vitro,” Cancer Research, 47:1278-81, 1987.

Tsuchida et al., “Gangliosides as tumor markers of human melanoma: bio-chemical and immunologic assays,” in New Horizons of Tumor Immunotherapy, Torisu and Yoshida (eds.), pp. 315-325, 1989.

Tuting et al., J. Immunol., 160:1139-47, 1998.

Ulmer et al, Science, 259:1745-49, 1993.

Vadhan-Raj et al., J Clin. Oncol., 6:1636, 1988.

van der Bruggen et al., “A gene encoding an antigen recognized by cytolytic T lymphocytes on ahuman melanoma,” Science, 264:716, 1991.

Verma et al., “Adjuvant Effects of Liposomes Containing Lipid A: Enhancement of Liposomal Antigen Presentation and Recruitment of Macrophages,” Infection and Immunity, 60:2438-44, 1992.

Vijayasaradhi et al., “The melanoma antigen gp75 is the human homologue of the mouse b (brown) locus gene product,” J Exp. Med., 171:1375, 1990.

Von Bultzingslowen, Pneumologie, 53:266-75, 1999.

Vosika et al., Cancer Immunol. Immunother., 18:107, 1984.

Watson, Ralph, Sarkar and Cohn, “Leukemia viruses associated with mouse myeloma cells,” Proc. Natl. Acad. Sci. USA, 66:344, 1970.

Westrick et al., Biochim. Biophys. Acta, 750:141-48, 1983a

Westrick et al., Cancer Res., 43:5890-94, 1983b

Whisler and Yates, “Regulation of lymphocyte responses by human gangliosides. I. Characteristics of inhibitory effects and the induction of impaired activation,” J. Immuunol., 125:2106-12, 1980.

Wide et al, in Radioimmunoassay Methods, Kirkham and Hunter (eds.), E. and S. Livingstone, Edinburgh, 1970.

Wilschut, “Preparation and properties of phospholipid vesicles,” in Methodologie des liposomes appliquee a la pharmacologie et a la biologies cellulaire, Leserman and Barbet (eds.), INSERM, Paris, pp. 1-10, 1982.

Yamaguchi et al., “Cell-surface antigens of melanoma recognized by human monoclonal antibodies,” Proc. Natl. Acad. Sci. USA, 84:2416-20, 1987.

Yamamoto et al., “In vitro Augmentation of Natural Killer Cell Activity and Production of Interferon-α/β and -γ with Deoxyribonucleic Acid Fraction from Mycobacterium bovis BCG,” Jpn. J Cancer Res., 79:866-73, 1988.

Yeh et al., “A cell-surface antigen which is present in the ganglioside fraction and shared by human melanomas,” Int. J. Cancer, 29:269-75, 1982.

Yin et al., “Effect of Various Adjuvants on the Antibody Response of Mice to Pneumococcal Polysaccharides,” Journal ofBiological Response Modifiers, 8:190-205, 1989.

Yokoyama et al., “Immunochemical studies with gangliosides,” J. Immunol., 90:372-80, 1963.

Zapata etal., Protein Eng., 8:1057-62, 1995.

Zitvogel et al., Nat. Med., 4:594-600, 1998.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. Accordingly, the exclusive rights sought to be patented are as described in the claims below:

What is claimed is:

1. A method of generating an immune or a T-cell response in an animal comprising administering to said animal a composition that comprises at least a first isolated peptide of from 9 to about 40 amino acids in length, or at least a first nucleic acid segment that encodes said peptide, wherein said peptide comprises a first contiguous amino acid sequence according to any one of SEQ ID NO: 1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326.

2. The method of claim 1, wherein said composition comprises at least a first isolated peptide of from 9 to about 35 amino acids in length, or at least a first nucleic acid segment that encodes said peptide.

3. The method of claim 2, wherein said composition comprises at least a first isolated peptide of from 9 to about 30 amino acids in length, or at least a first nucleic acid segment that encodes said peptide.

4. The method of claim 3, wherein said composition comprises at least a first isolated peptide of from 9 to about 25 amino acids in length, or at least a first nucleic acid segment that encodes said peptide.

5. The method of claim 4, wherein said composition comprises at least a first isolated peptide of from 9 to about 20 amino acids in length, or at least a first nucleic acid segment that encodes said peptide.

6. The method of claim 5, wherein said composition comprises at least a first isolated peptide of from 9 to about 15 amino acids in length, or at least a first nucleic acid segment that encodes said peptide.

7. The method of claim 6, wherein said composition comprises at least a first isolated peptide of from 9 to about 13 amino acids in length, or at least a first nucleic acid segment that encodes said peptide.

8. The method of claim 7, wherein said composition comprises at least a first isolated peptide of from 9 to about 11 amino acids in length, or at least a first nucleic acid segment that encodes said peptide.

9. The method of claim 9, wherein said composition comprises at least a first isolated peptide of from 9 to about 40 amino acids in length, or at least a first nucleic acid segment that encodes said peptide; said peptide comprising a first contiguous amino acid sequence according to any one of SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326.

10. The method of claim 9, wherein said composition comprises at least a first isolated peptide of from 9 to about 40 amino acids in length, or at least a first nucleic acid segment that encodes said peptide; said peptide comprising a first contiguous amino acid sequence according to any one of SEQ ID NO:28 to SEQ ID NO:311, SEQ ID NO:313, SEQ ID NO:314, SEQ ID NO:316 to SEQ ID NO:318.

11. The method of claim 10, wherein said composition comprises at least a first isolated peptide of from 9 to about 40 amino acids in length, or at least a first nucleic acid segment that encodes said peptide; said peptide comprising at least a first contiguous amino acid sequence selected from the group consisting of SEQ ID NO:34, SEQ ID NO:35, SEQ ID NO:49, SEQ ID NO:88, SEQ ID NO:144, SEQ ID NO:147, SEQ ID
NO:185, SEQ ID NO:198, SEQ ID NO:199, and SEQ ID NO:282.

12. The method of claim 11, wherein said composition comprises at least a first isolated peptide of from 9 to about 11 amino acids in length, or at least a first nucleic acid segment that encodes said peptide; said peptide consisting essentially of the amino acid sequence of any one of SEQ ID NO:1 to SEQ ID NO:4, SEQ ID NO: 13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326.

13. The method of claim 12, wherein said composition comprises at least a first isolated peptide of from 9 to about 11 amino acids in length, or at least a first nucleic acid segment that encodes said peptide; said peptide consisting essentially of the amino acid sequence of any one of SEQ ID NO:28 to SEQ ID NO:311, SEQ ID NO:313, SEQ ID NO:314, and SEQ ID NO:316 to SEQ ID NO:318.

14. The method of claim 13, wherein said composition comprises at least a first isolated peptide of from 9 to about 11 amino acids in length, or at least a first nucleic acid segment that encodes said peptide; said peptide consisting essentially of the amino acid sequence of SEQ ID NO:34, SEQ ID NO:35, SEQ ID NO:49, SEQ ID NO:88, SEQ ID NO:144, SEQ ID NO:147, SEQ ID NO:185, SEQ ID NO:198, SEQ ID NO: 199, or SEQ ID NO:282.

15. The method of claim 14, wherein said composition comprises at least a first isolated peptide that consists of the amino acid sequence according to any one of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326, or at least a first nucleic acid segment that encodes said peptide.

16. The method of claim 15, wherein said composition comprises at least a first isolated peptide that consists of the amino acid sequence according to any one of SEQ ID NO: 13 to SEQ ID NO:20, SEQ ID NO:28 to SEQ ID NO:311, SEQ ID NO:313, SEQ ID NO:314, and SEQ ID NO:316 to SEQ ID NO:318, or at least a first nucleic acid segment that encodes said peptide.

17. The method of claim 16, wherein said composition comprises at least a first isolated peptide that consists of the amino acid sequence of SEQ ID NO:34, SEQ ID NO:35, SEQ ID NO:49, SEQ ID NO:88, SEQ ID NO:144, SEQ ID NO:147, SEQ ID NO:185, SEQ ID NO:198, SEQ ID NO:199, or SEQ ID NO:282; or at least a first nucleic acid segment that encodes said peptide.

18. The method of claim 1, wherein said composition further comprises at least a second isolated peptide of from 9 to about 40 amino acids in length, or at least a first nucleic acid segment that encodes said peptide; said second peptide comprising at least a first contiguous amino acid sequence according to any one of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326.

19. The method of claim 18, wherein said composition further comprises at least a second isolated peptide of from 9 to about 40 amino acids in length, or at least a first nucleic acid segment that encodes said peptide; said second peptide comprising at least a first contiguous amino acid sequence according to any one of SEQ ID NO: 13 to SEQ ID NO:20, SEQ ID NO:28 to SEQ ID NO:311, SEQ ID NO:313, SEQ ID NO:314, and SEQ ID NO:316 to SEQ ID NO:318.

20. The method of claim 19, wherein said composition further comprises at least a second isolated peptide of from 9 to about 40 amino acids in length, or at least a first nucleic acid segment that encodes said peptide; said second peptide comprising at least a first contiguous amino acid selected from the group consisting of SEQ ID NO:34, SEQ ID NO:35, SEQ ID NO:49, SEQ ID NO:88, SEQ ID NO:144, SEQ ID NO:147, SEQ ID
NO: 185, SEQ ID NO: 198, SEQ ID NO: 199, and SEQ ID NO:282.

21. The method of claim 1, wherein said composition further comprises at least a third isolated peptide of from 9 to about 40 amino acids in length, or at least a first nucleic acid segment that encodes said peptide; said third peptide comprising at least a first contiguous amino acid sequence according to any one of SEQ ID NO:1 to SEQ ID NO:4, SEQ ID NO: 13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326.

22. The method of claim 21, wherein said third peptide comprises at least a first contiguous amino acid sequence according to any one of SEQ ID NO: 13 to SEQ ID NO:20, SEQ ID NO:28 to SEQ ID NO:311, SEQ ID NO:313, SEQ ID NO:314, and SEQ ID NO:316 to SEQ ID NO:318.

23. The method of claim 22, wherein said third peptide comprises at least a first contiguous amino acid selected from the group consisting of SEQ ID NO:34, SEQ ID NO:35, SEQ ID NO:49, SEQ ID NO:88, SEQ ID NO:144, SEQ ID NO:147, SEQ ID
NO:185, SEQ ID NO:198, SEQ ID NO:199, and SEQ ID NO:282.

24. The method of claim 1, wherein said composition comprises at least a first nucleic acid segment of from 27 to about 5000 nucleotides in length.

25. The method of claim 24, wherein said composition comprises at least a first nucleic acid segment of from 27 to about 3000 nucleotides in length.

26. The method of claim 25, wherein said composition comprises at least a first nucleic acid segment of from 27 to about 1000 nucleotides in length.

27. The method of claim 26, wherein said composition comprises at least a first nucleic acid segment of from 27 to about 500 nucleotides in length.

28. The method of claim 1, wherein said first nucleic acid segment is operably positioned under the control of at least a first heterologous promoter.

29. The method of claim 1, wherein said first nucleic acid segment is comprised within a vector.

30. The method of claim 29, wherein said first nucleic acid segment is comprised within a plasmid or viral vector.

31. The method of claim 1, wherein said composition further comprises a pharmaceutically acceptable excipient.

32. The method of claim 1, wherein said composition further comprises at least a first immunostimulant or at least a first adjuvant.

33. The method of claim 32, wherein said at least a first immunostimulant or said at least a first adjuvant preferentially enhances a T-cell response in a human.

34. The method of claim 32, wherein said at least a first immuno stimulant or said at least a first adjuvant is selected from the group consisting of Montanide ISA50, Seppic Montanide ISA720, a cytokine, a microsphere, a dimethyl dioctadecyl ammonium bromide adjuvant, AS-1, AS-2, Ribi Adjuvant, QS21, saponin, microfluidized Syntex adjuvant, MV, ddMV, an immune stimulating complex and an inactivated toxin.

35. The method of claim 1, wherein said animal is a human

36. The method of claim 35, wherein said human is a patient

37. The method of claim 1, wherein said composition is administered to a patient that has, is suspected of having, or is at risk for developing mesothelioma.

38. The method of claim 36, wherein said composition is administered to a patient that has, is suspected of having, or is at risk for developing malignant pleural mesothelioma.

39. The method of claim 1, wherein said composition is formulated for parenteral, intravenous, intraperitoneal, subcutaneous, intranasal, transdermal, or oral administration to said animal.

40. The method of claim 1, wherein said composition further comprises at least a first detection reagent.

41. The method of claim 40, wherein said detection reagent comprises a radiolabel, a spin label, or a fluorogenic, chromogenic, or a chemiluminescent label.

42. The method of claim 40, wherein said detection reagent specifically binds to a WT1 peptide, polypeptide, or antibody or antigen binding fragment specific therefor.

43. The method of claim 1, wherein said composition further comprises at least a second therapeutic agent for treating or preventing mesothelioma.

44. The method of claim 1, wherein said second therapeutic agent further comprises at least a second immunostimulant or at least a second adjuvant.

45. A method of inhibiting the biological activity of a WT1 polypeptide in a patient having, suspected of having, or at risk for developing malignant mesothelioma, comprising administering to said patient an effective amount of at least a first composition that comprises: (a) at least a first isolated peptide of from 9 to about 40 amino acids in length; (b) at least a first nucleic acid segment that encodes said peptide; (c) at least a first antibody or at least a first antigen binding fragment thereof having immunospecificity for said peptide; or (d) at least a first antigen-presenting cell or at least a first T cell that specifically binds to said peptide; wherein said peptide comprises a first contiguous amino acid sequence according to any one of SEQ ID NO:1 to SEQ ID NO:4, SEQ ID NO:13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326.

46. The method of claim 45, wherein inhibiting the biological activity of said WT1, polypeptide in said patient comprises administering to said patient an effective amount of at least a first composition that comprises: (a) at least a first isolated peptide of from 9 to about 40 amino acids in length, or (b) at least a first nucleic acid segment that encodes said peptide.

47. The method of claim 46, wherein inhibiting the biological activity of said WT1 polypeptide in said patient comprises administering to said patient an effective amount of at least a first composition that comprises: (a) at least a first isolated peptide of from 9 to about 40 amino acids in length, or (b) at least a first nucleic acid segment that encodes said peptide, and further wherein said peptide comprises at least a first contiguous amino acid sequence according to any one of SEQ ID NO:34, SEQ ID NO:35, SEQ ID NO:49, SEQ ID NO:88, SEQ ID NO:144, SEQ ID NO:147, SEQ
ID NO:185, SEQ ID NO:198, SEQ ID NO:199, and SEQ ID NO:282.

48. The method of claim 46, wherein inhibiting the biological activity of said WT1 polypeptide in said patient comprises administering to said patient an effective amount of at least a first composition that comprises: (a) at least a first antibody or at least a first antigen binding fragment thereof having immunospecificity for said peptide; or (b) at least a first antigen-presenting cell or at least a first T cell that specifically binds to said peptide, and further wherein said peptide comprises at least a first contiguous amino acid sequence according to any one of SEQ ID NO:34, SEQ ID NO:35, SEQ ID NO:49, SEQ ID NO:88, SEQ ID NO:144, SEQ ID NO: 147, SEQ ID NO:185, SEQ ID NO:198, SEQ ID NO:199, and SEQ ID NO:282.

49. A method of treating or preventing malignant mesothelioma in a patient having, suspected of having, or at risk for developing said mesothelioma, said method comprising administering to said patient a therapeutically-effective amount of at least a first composition that comprises: (a) at least a first isolated peptide of from 9 to about 40 amino acids in length; (b) at least a first nucleic acid segment that encodes said peptide; (c) at least a first antibody or at least a first antigen binding fragment thereof having immunospecificity for said peptide; or (d) at least a first antigen-presenting cell or at least a first T cell that specifically binds to said peptide; wherein said peptide comprises a first contiguous amino acid sequence according to any one of SEQ ID NO: 1 to SEQ ID NO:4, SEQ ID NO: 13 to SEQ ID NO:20, SEQ ID NO:28 to SEQ ID NO:311, SEQ ID NO:313, SEQ ID NO:314, SEQ ID NO:316 to SEQ ID NO:318, and SEQ ID NO:321 to SEQ ID NO:326, for a time sufficient to treat or prevent said malignant mesothelioma.

50. The method of claim 49, wherein treating said malignant mesothelioma in a patient having or suspected of having said mesothelioma, comprises administering to said patient a therapeutically-effective amount of at least a first composition that comprises: (a) at least a first isolated peptide of from 9 to about 40 amino acids in length; (b) at least a first nucleic acid segment that encodes said peptide; (c) at least a first antibody or at least a first antigen binding fragment thereof having immunospecificity for said peptide; or (d) at least a first antigen-presenting cell or at least a first T cell that specifically binds to said peptide; wherein said peptide comprises a first contiguous amino acid sequence according to any one of SEQ ID NO:34, SEQ ID NO:35, SEQ ID NO:49, SEQ ID NO:88, SEQ ID NO:144, SEQ ID NO:147, SEQ ID NO:185, SEQ ID NO:198, SEQ ID NO:199, and SEQ ID NO:282, for a time sufficient to treat said malignant mesothelioma.
